SEQUENCE LISTING (2 sequences)

SEQ ID NO: 1 - DNA, 1520 bp, Homo sapiens
gcggccgcgaattcggcacgagcggcgcacaagatggcggctctggcggcctaaagaaggcggccgcggctcagccgtgggctctaacgcggggctgggggccggagacagacttcgcccaggtgacgggtagtaggggcggcgccgcttggcctcgtggggtgtaagacccacttgctgttgcccccggaccttgccgccacaccagccctgtcctggggcggaaccgaaggaaggtcgggccctgctgccccgccccgtccttcctccttcccgggcggtcactgtgcgtggctcacttttagagtttacttcaaccacgtggagcttccatggcggcctctcaggtcctgggggagaagattaacatcctgtcgggagagactgtcaaagctggggacagggacccgctggggaacgactgtcccgagcaagataggctcccccagcgctcctggaggcagaagtgtgcctcctacgtgttggccctgaggccctggagcttcagtgcctcactcacaccggtggccctgggcagtgcccttgcctacagatcccacggtgtcctggatcccaggctcttggtgggttgtgccgtggctgtcctggctgtgcacggggccggtaatttggtcaacacttactatgacttttccaagggcattgaccacaaaaagagtgatgacaggacacttgtggaccgaatcttggagccgcaggatgtcgtccggttcggagtcttcctctacacgttgggctgcgtctgtgccgcttgcctctactacctgtcccctctgaaactggagcacttggctcttatctactttggaggcctgtctggctcctttctctacacaggaggaattggattcaagtacgtggctctgggagacctcatcatcctcatcacttttggcccgctggctgtgatgttcgcctacgccatccaggtggggtccctggccatcttcccactggtctatgccatccccctcgccctcagcaccgaggccattctccattccaacaacaccagggacatggagtccgaccgggaggctggtatcgtcacgctggccatcctcatcggccccacgttctcctacattctctacaacacactgctcttcctgccctacctggtcttcagcatcctggccacacactgcaccatcagcctggcactccccctgcttaccattcccatggccttctcccttgagagacagtttcgaagccaggccttcaacaaactgccccagaggactgccaagctcaacctcctgctgggacttttctatgtctttggcatcattctggcaccagcaggcagtctgcccaaaatttaaggggacaagtagctccccccacgacatgtctccctttcttagaatatattaaagtcagagtctctgaggaaggaatgtgatttggcagtcagggtactaagcatgggtgggaactcctgccttataaaaattgtttttgtgttcttaaagataaaaaaaaaaaaaaaaaaa

SEQ ID NO: 2 - protein, 338 aa, Homo sapiens
MAASQVLGEKINILSGETVKAGDRDPLGNDCPEQDRLPQRSWRQKCASYVLALRPWSFSASLTPVALGSALAYRSHGVLDPRLLVGCAVAVLAVHGAGNLVNTYYDFSKGIDHKKSDDRTLVDRILEPQDVVRFGVFLYTLGCVCAACLYYLSPLKLEHLALIYFGGLSGSFLYTGGIGFKYVALGDLIILITFGPLAVMFAYAIQVGSLAIFPLVYAIPLALSTEAILHSNNTRDMESDREAGIVTLAILIGPTFSYILYNTLLFLPYLVFSILATHCTISLALPLLTIPMAFSLERQFRSQAFNKLPQRTAKLNLLLGLFYVFGIILAPAGSLPKI

(19) United States
(12) Patent Application Publication
(10) Pub. No.: US 2010/0233700 A1
(43) Pub. Date: Sep. 16, 2010

(54) UBIAD1 GENE AND HYPERLIPIDEMIA

(75) Inventor: JAYNE S WEISS, Bloomfield, MI (US)

Correspondence Address: C. Rachal Winger;c/o OCIP Group, 1900 Main Street, Suite 600, Irvine, CA, 92614-7319, US

(73) Assignee: WAYNE STATE UNIVERSITY, Detroit, MI (US)

(21) Appl. No.: 12/649,087

(22) Filed: Dec. 29, 2009

Related U.S. Application Data
(63) Continuation-in-part of application No. PCT/US2008/069262, filed on Jul. 3, 2008.
(60) Provisional application No. 60/953,893, filed on Aug. 3, 2007.
(60) Provisional application No. 60/948,361, filed on Jul. 6, 2007.

(51) Int. Cl. C12Q 1/68; C07H 21/04

(52) U.S. Cl. 435/6; 536/23.1

(57) ABSTRACT

The disclosure relates to genetic mutations in UBIAD1 gene that segregate with Schnyder's crystalline corneal dystrophy. The disclosure provides methods for detecting such mutations as a diagnostic for Schnyder's crystalline corneal dystrophy either before or after the onset of clinical symptoms. Also provided are screening methods for identifying medical conditions related to cholesterol metabolism, including atherosclerosis, risk of future loss of vision, and future need for corneal transplantation.

### CROSS-REFERENCES TO RELATED APPLICATIONS

This application is a continuation-in-part of International Application No. PCT/US2008/069262 filed on Jul. 3, 2008, which is a non-provisional of U.S. Provisional Application No. 60/953,893 filed on Aug. 3, 2007 and U.S. Provisional Application No. 60/948,361 filed on Jul. 6, 2007, and U.S. Provisional Application No. 60/xxx,xxx filed on Jul. 3, 2007, which applications are incorporated by reference in their entirety herein.

### STATEMENT OF RIGHTS TO INVENTIONS MADE UNDER FEDERALLY SPONSORED RESEARCH

This invention was funded in part by grants and contracts from the National Eye Institute of National Institutes of Health (grant EY12972) which provides to the United States government certain rights in this invention. Additional funding is provided by the National Cancer Institute, NIH, under Contract No. HHSN261200800001E.

### REFERENCE TO SEQUENCE LISTING, TABLES OR COMPUTER PROGRAM LISTING

Incorporated by reference in its entirety herein is a computer-readable nucleotide/amino acid sequence listing submitted concurrently herewith and identified as follows: One 680 MB ASCII (Text) file named “2066728-00002 Sequence Listing.ST25.txt,” created on Dec. 29, 2009.

### FIELD OF THE DISCLOSURE

The present disclosure relates to the field of genetics and medicine, specifically to a gene UbiA prenyl-transferase Domain containing 1 (UBIAD1), the mutation of which results in hyperlipidemia, and contributes to the causation of Schnyder's crystalline corneal dystrophy (SCCD).

### BACKGROUND OF THE DISCLOSURE

The disease Schnyder's Crystalline Corneal Dystrophy (SCCD) was renamed Schnyder Corneal Dystrophy (SCD) in 2008. Consequently, both terms are used in this application. (Weiss, et al., Cornea 2008; 27 Supp 2:S1-83). SCCD (OMIM 121800) was initially described by van Went and Wibaut in the Dutch literature in 1924, when they reported characteristic corneal changes in a three generation family. (van Went, et al., Niederl Tijdschr Geneesks 1924; 68:2996-2997). Subsequently, in 1929, a Swiss ophthalmologist named Schnyder published a report of the same disease in a different three generation family. (Schnyder, Schweiz Med Wschr 1929; 10:559-571; Schnyder, Klin Monatsbl Augenheilkd 1939; 103:494-502). The autosomal dominant disease became known as SCCD and is characterized by the abnormal deposition of cholesterol and phospholipids in the cornea. (Rodrigues, et al., Am J Opthalmol 1987; 104:157-163). The resultant progressive bilateral corneal opacification leads to decreasing visual acuity.

SCCD is considered to be a rare dystrophy, with less than 150 articles in the published literature, and most articles reporting only a few affected individuals. In the late 1980's, Weiss identified four large Swede-Finn pedigrees of patients with SCCD in central Massachusetts and published the results of clinical exams of 33 affecteds. (Weiss, Cornea 1992; 11:93-10; Weiss, Opthalmology 1996; 103:465-473). At the same time the four pedigrees were being examined clinically, an effort was also begun to define the genetic mutation in the disease. Additional families with SCCD were recruited nationally and internationally. Using two of the original Swede-Finn pedigrees, a genome-wide DNA linkage analysis mapped the SCCD locus within a 16 cM interval between markers D1S2633 and D1S228 on chromosome 1p367. In a subsequent study, a total of 13 pedigrees was used to perform haplotype analysis using densely spaced microsatellite markers refining the candidate interval to 2.32 Mbp between markers D1S1160 and D1S1635. A founder effect was implied by the common disease haplotype which was present in the initial Swede-Finn pedigrees. Identity by state was present in all 13 families for two markers, D1S244 and D1S3153, further narrowing the candidate region to 1.57 Mbp. (Riebeling, et al., Opthalmologe 2003; 100:979-983; Theendakara, et al., Hum Genet. 2004; 114:594-600). Several candidate gene analyses have been preformed for mutations by sequencing the exonic regions of ENOL, CA6, LOC127324, SLC2A5, SLC25A33, PIK3CD, CLSTN1, CTNNBIP1, LZIC, NMNAT, RBP7, UBE4B, K1F1B, PGD, CORT, DFFA, and PEX14. (Aldave, et al., Mol. Vis. 2005; 11:713-716). However, no pathogenic mutations were found. In May 2007, Oleynikov and coworkers reported results of mutation screening of the remaining 16 of the 31 genes that were within the 2.32 Mbp candidate region for SCCD on the short arm of chromosome. (Oleynikov, et al., ARVO Poster 2007; 549; van Went, et al., Niederl Tijdschr Geneesks 1924; 68:2996-2997). They found no disease causing mutations in SCCD patients.

The possible explanations for not finding mutations in any of the 31 genes studied included locus heterogeneity for SCCD, incomplete gene annotation for the candidate interval, the presence of pathogenic mutations outside the coding regions of candidate genes, or an error in the assignment of the candidate locus for SCCD due to misclassifications of disease status in family members.

Re-analysis of the pedigrees reported in the article by Theendakara et al., indeed showed a misclassification in one individual. (Theendakara, et al., Hum Genet. 2004; 114:594-600). Individual III-5 in Family 9 was reported by herself and her father to not have SCCD. Re-review of the patient's clinical chart, however, revealed that she had evidence of subtle SCCD without crystals. The phenotype in the patient's family was atypical with some affecteds having had only a diffuse, confluent corneal clouding without crystal deposition. (Weiss, Trans Am Opthalmol Soc 2007; 105: 616-648).

In the article by Weiss detailing the phenotypic variations and long term visual morbidity in 4 pedigrees with SCCD, Family 9 was identified as Family J. When compared with the corneal findings in other SCCD families, the dystrophy phenotype in Family 9 appeared to be milder resulting in less visual morbidity than in other SCCD pedigrees. Affecteds in Family 9 often maintained excellent visual acuity well into old age. (Weiss, Trans Am Opthalmol Soc 2007; 105:616-648). Family 9 had been used to define the centromeric boundary of the candidate interval at D1S16358. (Theendakara, et al., Hum Genet. 2004; 114:594-600).

It was decided to remove Family 9 from the analysis and re-evaluate the haplotypes using only the other 12 families. This resulted in a shift of the centromeric boundary of the candidate interval from D1S1635 to D1S2667. The expanded candidate interval included C1orf127, TARDBP, MASP2, SRM, EXOSC10, FRAP1, ANGPTL7, UBIAD1 and LOC39906.

### SUMMARY OF THE DISCLOSURE

The present inventor chose three genes for initial examination: ANGPTL7, FRAP1 and UbiA prenyl-transferase Domain containing 1 (UBIAD1). ANGPTL7 and UB1AD1 were included in the study because both were expressed in the cornea. FRAP1 and UBIAD1 were included because of their involvement in lipid metabolism, diabetes and nutrient signaling. (Parent, et al., Cancer Res 2007; 67:4337-4345; McGarvey, et al., Oncogene 2001; 20:1042-1051; McGarvey, et al., Prostate 2003; 54:144-155; McGarvey, et al., J Cell Biochem 2005; 95:419-428; and Peek, et al., Invest Opthalmol Vis Sci 1998; 39:1782-1788).

The present disclosure is directed in part to the identification of the UBIAD1 gene as the cause of the hereditary eye disease Schnyder's crystalline corneal dystrophy (SCCD). This information is useful for treatment of lipid abnormalities.

One embodiment of the present disclosure includes an isolated polynucleotide having the nucleotide sequence of or which is complementary to at least a portion of the UBIAD1 gene of SEQ ID NO:1, wherein the nucleotide sequence contains at least one gene mutation which correlates with the risk of SCCD and where at least one gene mutation is located at the codon corresponding to amino acid position 97, 118, 121, 122, 171, 177, 186, 236 or 240 of SEQ ID NO:2, and where the mutation causes a change in the amino acid encoded by that codon, with the proviso that the codon corresponding to amino acid position 121 of SEQ ID NO:2 does not encode valine. In another embodiment, the change in the amino acid is a nonconservative change. In yet another embodiment, the polynucleotide is labeled with a detectable agent. In yet another embodiment, the polynucleotide has between 10 and 40 consecutive nucleotides. In yet another embodiment, the mutation results in a Ala97Thr, Asp118Gly, Leu121Phe, Val122Gly, Val122Glu, Ser171Pro, Gly177Arg, Gly186Arg, Leu188His, Asp236Glu, or Asp240Asn-substitution.

Embodiments disclosed herein also include methods for determining whether a subject is at risk for developing SCCA by obtaining a biological sample from the subject; determining the presence or absence of one or more gene mutations of the UBIAD1 gene of SEQ ID NO:1 where the gene mutation is located at the codon corresponding to amino acid positions 97, 118, 121, 122, 171, 177, 186, 188, 236 or 240 of SEQ ID NO:2; and determining if the gene mutation results in a change in the amino acid where the presence of the gene mutation resulting in a change in the amino acid indicates that the subject is at risk for developing SCCD. In another embodiment, the change in the amino acid is a non-conservative change. In yet another embodiment, determining the presence or absence of the gene mutation involves the step of amplification of at least a portion of the nucleic acid using one or more pairs of oligonucleotide primers flanking at least one of the codons corresponding to amino acid position 97, 118, 121, 122, 171, 177, 186, 236 or 240. In yet another embodiment, the gene mutation results in a Ala97Thr, Asp118Gly, Leu121Phe, Val122Gly, Val122Glu, Ser171Pro, Gly177Arg, Gly186Arg, Leu188His, Asp236Glu, or Asp240Asn substitution.

Embodiments disclosed herein also include methods for determining the presence or absence of one or more gene mutations of the UBIAD1 gene of SEQ ID NO:1 by obtaining a biological sample from the subject; determining the presence or absence of one or more gene mutations of the UBIAD1 gene of SEQ ID NO:1 that creates a risk factor for a disease and/or a disease wherein the at least one gene mutation is located at the codon corresponding to amino acid position 97, 118, 121, 122, 171, 177, 186, 188, 236 or 240 of SEQ ID NO:2; and determining if the gene mutation results in a change in the amino acid wherein the presence of one or more gene mutations resulting in a change in the amino acid indicates the presence of the risk factor for a disease and/or the disease.

In another embodiment, the change in the amino acid is a non-conservative change. In yet another embodiment, determining the presence or absence of the gene mutation further involves using one or more pairs of oligonucleotide primers flanking at least one of the codons corresponding to amino acid position 97, 118, 121, 122, 171, 177, 186, 236 or 240. In yet another embodiment, the gene mutation results in a Ala97Thr, Asp118Gly, Leu121Phe, Val122Gly, Val122Glu, Ser171Pro, Gly177Arg, Gly186Arg, Leu188His, Asp236Glu, or Asp240Asn substitution.

In another embodiment, the methods for determining the presence or absence of one or more gene mutations of the UBIAD1 gene of SEQ ID NO:1 is used for diagnosing SCCD in a subject. In another embodiment it is used for determining whether a subject is at risk for developing atherosclerosis. In yet another embodiment, it is used for determining whether a subject is at risk for developing loss of vision. In yet another embodiment, it is used for determining whether a subject is at risk for requiring future corneal transplant. In yet another embodiment, it is used for determining whether a subject is at risk for developing SCCD.

Embodiments disclosed herein also include methods of screening for an effect of a mutation in the UBIAD1 gene in cholesterol metabolism by providing an aliquot of a purified protein that is involved in cholesterol metabolism; contacting the aliquot with a non-mutant protein encoded by the UBIAD1 gene of SEQ ID NO:1; determining the amount of the non-mutant protein that is bound to the purified protein; contacting a second aliquot of the purified protein with a mutant protein encoded by a mutant UBIAD1 gene; determining the amount of mutant protein encoded by the mutant protein that is bound to the purified protein and then comparing the amount of non-mutant protein bound to the purified protein with the amount of mutant protein bound to the purified protein, the difference in amounts indicating that the mutation in UBIAD1 can be involved in cholesterol metabolism.

In another embodiment, the protein involved in cholesterol metabolism is apolipoprotein A-I, apolipoprotein A-II, apolipoprotein E, apolipoprotein B, or HMG-CoA reductase. In yet another embodiment, the screening is performed to determine the presence of a risk factor for atherosclerosis. In yet another embodiment, the screening is performed to determine the presence of atherosclerosis.

This is the first discovery of the causative gene in SCCD. This disclosure is more generally applicable to lipid storage in the cornea and lipid metabolism elsewhere in the body and diseases and conditions associated therewith.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Family Q from the United States. Individuals whose DNA was used for DNA sequencing are marked with an asterisk. Individual III-12, a 19-year-old woman, did not have corneal crystal deposition on clinical examination but had trace haziness of the cornea. It was not clear whether she had the disease phenotype because of the minimal corneal changes but genotyping demonstrated that this individual carried the disease haplotype.

FIG. 2: Family T from the United States. Individuals whose DNA was used for DNA sequencing are marked with an asterisk.

FIG. 3: Family Y from Germany. Individuals whose DNA was used for DNA sequencing are marked with an asterisk.

FIGS. 4A-B: DNA sequencing of UbiA prenyl-transferase Domain containing 1 (UBIAD1) exons in SCCD patients revealed non-synonymous mutations. Each panel contains a chromatogram from an unaffected individual (top). A: Two families, Y (patient II-1, middle) and Q (patient II-11, bottom) share the same mutation, an A305G that alters codon AAC to AGC and changes the amino acid at position 102 (N102S). B: Family T, patient III-3 (bottom) has a G529C which changes glycine at position 177 to arginine (G177R).

FIGS. 5A-F: Summary of transcripts in UBIAD1 locus (Gene ID: 29914). A: RefSeq curated transcript representing best available data (RefSeq NM_{—}013319); B—F: transcripts that are possible based on alignment of spliced ESTs. Transcript E can represent alternative promoter usage, rather than alternative splicing. Mutations were found in exon 1 of transcript A (RefSeq NM_{—}013319). Exons are numbered 1 to 5 beginning at the transcription start site.

FIG. 6: Transcript A (see FIG. 5; RefSeq NM_{—}013319) encodes a protein of 338 amino acids. Transmembrane spanning regions (dark grey) are labeled 1-8 and correspond to amino acids 83-103, 134-154, 160-180, 188-208, 209-229, 245-267, 277-297, and 315-335. The prenyltransferase domain is indicated by the horizontal line at top and comprises amino acids 58-333 the top. Locations of the two SCCD mutations identified in this study are indicated below the protein.

FIG. 7: A patient with central corneal crystals: Individual II-10 in Family Q is a 43-year-old male with central corneal crystals, mid peripheral haze and arcus lipoides. Best corrected visual acuity (BCVA) was 20/50.

FIG. 8: Diagram of corneal changes with age which occur in SCCD. Initial corneal opacification occurs centrally and paracentrally, followed by formation of peripheral acrus lipoids and finally mid-peripheral corneal haze. With increasing corneal opacification there is a loss of visual acuity and decrease in corneal sensation.

FIG. 9: Chromatogram showing the mutation D240N. FIG. 9 (top) shows that the amino acid at position 240 is D, conserved across a range of species. FIG. 9 (bottom) shows N at position 240 in a human sample.

FIG. 10: Nucleotide sequence of UBIAD1.

FIG. 11: Amino Acid sequence of UBIAD1.

FIGS. 12A-B: Family G originating from the United States affected with SCCD. A: Pedigree with blackened symbols representing affected individuals. Individuals whose DNA was used for DNA sequencing are marked with an asterisk. B: Sequence chromatogram showing the G186R mutation in exon 2 from patient II-6 (top). A chromatogram from a healthy individual is shown for comparison (bottom).

FIGS. 13A-B: Family J originating from the United States with known Hungarian ethnicity affected with SCCD. A: Pedigree with blackened symbols representing affected individuals. Individuals whose DNA was used for DNA sequencing are marked with an asterisk. B: Sequence chromatogram showing T1751 mutation in exon 1 from patient III-11 (top). A chromatogram from a healthy individual is shown for comparison (bottom).

FIGS. 14A-D: Analysis of the UBIAD1 protein. A: Locations of Familial SCCD mutations on the annotated, linear UBIAD1 protein. Green arrowheads: mutations reported in this publication. Black: mutations reported in Weiss, Trans Am Opthalmol Soc 2007; 105: 616-648; Blue: mutations reported in Orr, et al., PLoS ONE 2007; 2:e685. The location of the S75F single nucleotide polymorphisms (SNP) is indicated by a red arrowhead. Predicted domains are labeled as described in the Examples. B: Protein structure in the membrane. Black residues are mutated in SCCD families; Orange: regions outside the prenyl transferase domain. Blue: acidic residues. Red: basic residues. HRM, heme regulatory motif (box): CxxC: oxido-reductase motif (CAAC, circled). The location of the S75F polymorphism is indicated (green). Three clusters of mutations are circled (Loops 1, 2, and 3). C: Sequence alignment of the putative ligand: polyprenyldiphosphate binding site in Loop 1. The locations of mutated residues seen in SCCD patients, N102S and D112G are indicated. D: Relationship between various prenyltransferase proteins.

FIGS. 15A-B: Slit-lamp photographs of the cornea demonstrating a pattern of central corneal crystalline deposition with a denser scalloped border, accompanied by mid-peripheral haze and arcus lipoides. Two affected individuals with different SCCD mutations demonstrate virtually identical corneal findings. A: Slit-lamp photograph of the cornea from a 42-year-old African American woman with SCCD from family FF with the D236E mutation. B: Slit-lamp photograph of the cornea from a 70-year-old German man with SCCD from family K1 with the S171P mutation.

FIGS. 16A-B: Slit-lamp photographs of the cornea demonstrating different patterns of corneal opacification from affected individuals from two different SCCD families with the G177R mutation. A: Slit-lamp photograph of the cornea of a 38-year-old Taiwanese woman from family X with dense corneal opacification more prominent centrally and peripherally and with central corneal crystalline deposition. B: Slit-lamp photograph of the cornea of a 39-year-old man from Kosovo from family Z with prominent corneal crystalline deposition and less prominent corneal opacification.

FIG. 17: Slit-lamp photograph of the cornea from a 74-year-old Caucasian man with SCCD, patient II-3, from family J. The patient had unusually good best-corrected visual acuity of 20/25 with diffuse corneal haze and no evidence of crystalline deposits.

FIGS. 18A-D: Patient corneas and UBIAD1 sequencing of SCD probands. Corneal photos (top) and patient forward and reverse chromatograms (bottom) are shown above a wild type sequence. A: Family GG with a novel A97T mutation. External photograph of the cornea demonstrating central and paracentral crystalline deposition in a 36 year old male. Proband sequences from two independent PCR products are shown over wild type. B: Family AA, a first SCD family of Native American ancestry with a novel V122E mutation. External photograph of the cornea demonstrating central and paracentral crystalline deposits, diffuse corneal haze, and arcus lipoides in a 69 year old male (top). C: Family KK with a ‘hotspot’ N102S mutation. External photograph of the cornea demonstrating central crystalline deposit, mid peripheral haze, and arcus lipoides in a 61 year old male. D: Family LL with a novel D112N mutation. External photograph of the cornea demonstrating paracentral crystalline deposition in a 25 year old male.

FIGS. 19A-C: Highly conserved UBIAD1 residues are mutated in SCD. A: Locations of 17 amino acids mutated in SCD patients are indicated by arrows. 15 out of 17 residues were universally conserved from sea urchins to human. The height of bars in the graph below the sequence alignment (grey) is an indicator of degree of conservation. The taller the bar the higher the degree of conservation. Alignment was performed using ClustalX 2.0.11.15. B: Three of four new SCD alterations are universally conserved across species from sea urchin to human. Regions of alignment of UBIAD1 homologs from the species indicated (left) encompassing human SCD mutations: A97, D112, V122, L188, are shown. Positions of mutant amino acids are indicated and mutations in SCD are shown after amino acid position. Alignment was performed using ClustalX 2.0.11 and the position of the human protein in the alignment is indicated on the left (box). C: Evolutionary relationships based upon UBIAD1 homology. Calculations were made using ClustalX 2.0.11.

FIGS. 20A-B: Locations of familial SCD alterations in UBIAD1. A: A linear diagram of the UBIAD1 protein allows identification of a mutation hotspot (N102S). Each arrow represents a mutation in a putative unrelated family. Locations of new families presented in this study are indicated (green arrows). Previously published SCD mutations are also indicated (black arrows). Predicted transmembrane domains (n=8) are indicated by grey boxes and numbered (bottom). Location of the prenyl-transferase domain is shown from amino acids 58 to 333 (horizontal line, bottom). A previously described S75F SNP (red arrow) is also indicated. B: Locations of SCD mutations in a proposed 2-D model of UBIAD1 in a lipid bilayer. Solid Black: residues mutated in SCD families, Orange: amino acids outside the prenyl-transferase domain, Blue: acidic residues, Red: basic residues, HRM: heme regulatory motif (box), CxxC: oxidoreductase motif (CAAC, small circle), Green: S75F polymorphism. Three clusters of mutations are circled (Loops 1, 2, and 3).

FIG. 21A-F: Cellular localization of wild type human UBIAD1. Co-localization within cultured normal human keratocytes of UBIAD1 and protein disulfide isomerase, an enzyme in endoplasmic reticulum, is shown in panels A-C. Co-localization of UBIAD1 and OXPHOS complex I, an enzyme in mitochondria, is shown in D-F. UBIAD1 labeling is red (B and E). Protein disulfide isomerase and OXPHOS I are green (A and D). UBIAD1 did not co-localize with the endoplasmic reticulum (C), but did co-localize with mitochondria (co-localizing red and green show as orange in F). Bar is 50 μm and applies to all.

FIG. 22A-F: Localization of SCD mutant UBIAD1. Co-localization of UBIAD1 and OXPHOS complex I mitochondrial marker in keratocytes derived from the Family KK proband (N1025 mutation, panels A-C) and a healthy donor (D-F). UBIAD1 (red, A and D) and a mitochondrial marker (green, B and E) show co-localization (orange) in both normal (F) and SCD disease keratocytes (C). Bar is 25 μm and applies to all.

FIGS. 23A-F: Three dimensional modeling of human UBIAD1. A: Alignment of E. coli UbiA and human UBIAD1 together with predicted transmembrane helices (pred), H=helix, I=inside, o=outside. The predicted transmembrane helices of both models are highlighted as bold italics font, underlined are the amino acid residues identified as potentially responsible for the complexation of organic diphosphate. B: Rainbow representation of the side view of a putative 3D-structure of UBIAD1 in the membrane. Approximate location of the lipid bilayer is indicated (horizontal lines). Inside and outside are arbitrary labels of membrane sidedness. Green spheres represent magnesium cations in the active site with a docked farnesyl-diphosphate (red stick representation). The side chain of N102 is shown as a space-filled atom. C: A top view of a potential 3D structural model by protein threading presented as described in FIG. 23B. Spacefill atoms indicate the location of N102 hotspot amino acid, green dot is Mg²⁺, and magenta atoms show potential binding of a putative substrate. D: Representation of a hypothetical docking arrangement of farnesyldiphosphate and a 1,4-dihydroxy aryl compound as a potential aromatic substrate in the model active site of UBIAD1. The hypothetical aromatic substrate is recognized by N102 (arrow) and R235 via hydrogen bonds and by hydrophobic interactions with P64. The distance of the C2-atom of the hydroquinone to the C1-atom of the farnesyl moiety is 3.8 Å (red dashed line), which would allow prenylation at C2 of the substrate. Green spheres are Mg²⁺ ions required for diphosphate activation. E: Docking arrangement of the two putative substrates (23D and E) in the proposed active site of UBIAD1 mutated from an asparagine at position 102 to a serine (arrow), similar to mutations found in 41% of SCD families. The aromatic substrate is no longer recognized by N102, but by S69 and, as before, by R235 via hydrogen bonds and by hydrophobic interactions with P64. C2 of the aromatic substrate is no longer positioned correctly to allow prenylation. Green spheres: Mg²⁺ ions. F: A putative active site of UBIAD1 is shown with a putative substrate that optimally docks to the protein, a menaquinone-farnesyl derivative. Substrates with longer fatty acid tails were also successfully docked. The interaction is stabilized by hydrogen bonds (dashed lines) with N₁₀₂ and 8235. N102 is frequently mutated in SCD. R235 can be influenced by neighboring residues, N232, N233, and D236, which cause SCD when altered. The quinone moiety and farnesyl chain are recognized by P64, F107, and other indicated residues via hydrophobic interactions.

FIGS. 24A-B: Locations of selected SCD alterations in model. A: Sideview of UBIAD1 showing locations of wild type amino acids mutated in SCD. B: Top view. In each view, only several residues mutated in SCD are visible. Farnesyldiphosphate is shown as a stick representation. The side chains of SCD mutations reported herein are shown as spacefilled atoms: A97, N102, D112, V122, L188.

FIG. 25: SCD family N pedigree. Affected individuals are shown in black, unaffected, no color. Family members examined by UBIAD1 sequencing are indicated with an asterisk.

FIG. 26. SCD Family F1 Pedigree. Affected individuals are shown in black. Asterisks indicate patients examined by UBIAD1 sequencing.

FIG. 27: Key enzymatic prenylation reaction catalyzed by UbiA during biosynthesis of ubiquinone. Prenylation of 4-hydroxybenzoic acid by oligoprenyl diphosphates are shown (n>1). A two substrate reaction is shown similar to that proposed for human UBIAD1.

FIG. 28: Docking simulation with naphthalinediol as a putative substrate. Tertiary protein structure model of human UBIAD1 with eight transmembrane helices and a putative naphthalinediol substrate docked (shown as a spacefill atom representation).

FIGS. 29A-B: Models showing locations of Loops 1-3 containing clusters of SCD mutations. See FIG. 20B for comparison, to identify SCD mutations in each loop. Two views are shown, a side view (left side) and top view (right side). These highlight the loop regions containing amino acids implicated in SCD. Loop 1 (containing amino acids A97 to R132) is shown in orange, loop 2 (Y174 to A184) in blue, and loop 3 (L229 to S257) in green. Mutated S102 is shown as a spacefill atom and a docked farnesyldiphosphate is shown as a stick representation (red).

FIGS. 30A-C: Structures of potential substrates successfully docked with the UBIAD1 model. (A) Farneslydiphosphate (C₁₅H₂₅O₇P₂⁻³). (B) Menaquinone (C₁₁H₈O₂). (C) Naphthalenediol (C₁₀H₈O₂).

FIG. 31A-C: Corneal diagram of location of corneal changes. Initial changes are noted in central cornea (A) with occurrence of corneal crystals and/or central haze followed by formation of (C) arcus lipoides and finally mid peripheral stromal haze (B). (From Weiss, et al., Opthalmology 1992; 99:1072-1081).

FIG. 32: Map of Finland with arrows pointing to towns with patients identified to have SCCD.

FIG. 33: Pedigree A: Patients who have had penetrating keratoplasty (PKP) are indicated. Individual patients are identified by a roman numeral representing the family generation and an Arabic number. The unique patient identifier number and pedigree name is used to identify the patient in the text, photographs and tables.

FIG. 34: Pedigree B: Key for this figure is listed in FIG. 33. Individual patients are identified by a roman numeral representing the family generation and an Arabic number. The unique patient identifier number and pedigree name is used to identify the patient in the text, photographs and tables. Patients who have had PKP are indicated.

FIG. 35: Pedigree J: Key for this figure is listed in FIG. 33. Individual patients are identified by a roman numeral representing the family generation and an Arabic number. The unique patient identifier number and pedigree name is used to identify the patient in the text, photographs and tables. Patients who have had PKP or phototherapeutic keratectomy (PTK) are indicated.

FIG. 36: Visual acuity flow chart of patients with SCCD.

FIG. 37: Regression analysis of BCVA with age in years (yrs.) in SCCD patient who have no other ocular pathology. Y axis represents log MAR visual acuity and X axis represents age y=−0.033+0.002x; R²=0.046.

FIGS. 38A-C: The corneas of a 28 year old female in family G, with uncorrected visual acuity (UCVA) 20/15 right eye (OD) and 20/20 left eye (OS) which demonstrate an almost complete circle of crystalline deposition which appears to be bilaterally symmetric. OD and OS appear to have a mirror image crystalline deposit. A: External photograph of OD. B: External photograph of OS. C: Slit lamp photograph demonstrating subepithelial crystalline deposits.

FIG. 39: External photograph of the cornea of a 14 year old male, III 2, in family B, with UCVA of 20/20 and partial arc deposition of subepithelial crystals. A symmetrical mirror image crystalline deposit was seen in the other eye.

FIG. 40: External photograph of the cornea of a 38 year old male, II 7, in family A, with central haze, central ring of crystals, mid peripheral clouding and arcus lipoides. BCVA was 20/25.

FIG. 41: External photograph of the cornea of a 37 year old male, III 5, in family B, with central plaque of subepithelial crystals in visual axis and BCVA of 20/50. Six months later, PRK/PTK was performed with improvement of UCVA to 20/25

FIG. 42: Slit lamp photograph of the cornea of a 23 year old female, III 9, in family B, with BCVA 20/20 and central corneal ring opacity slightly inferiorly displaced in the visual axis. No subepithelial crystals were present.

FIG. 43: External photograph of the cornea of a 40 year old male, II 5, in family A, with BCVA 20/25 and central disc shaped stromal opacity and arcus lipoides. The central opacity is panstromal and is slightly inferiorly displaced in the visual axis. No subepithelial crystals were present.

FIG. 44: Slit lamp photograph of the cornea of a 47 year old male, II 1, in family B, with BCVA 20/30. Retro illumination reveals the central opacity is more lucent in its middle and the opacity appears to be tessellated. Mid peripheral haze and prominent arcus lipoides are also noted.

FIG. 45: External photograph of the cornea OD of a 63 year old female, I 1, in family B, with BCVA 20/70 with subepithelial crystals, diffuse corneal haze and arcus lipoides. OD underwent PKP, cataract extraction (CE) and intraocular lens (IOL) surgery within the year.

FIG. 46: External photograph of the cornea of a 72 year old female in family C, patient number 2, with BCVA 20/40 with dense central opacity, mid peripheral haze and arcus lipoides that underwent PKP, CE and IOL within the year.

FIG. 47: External photograph of the cornea of a 74 year old male, I 1, in family J, with BCVA 20/25 and diffuse corneal opacification and arcus lipoides.

FIGS. 48A-B: A: External photograph of the cornea of a 39 year old female, II 2, in family B, with BCVA of 20/20 with diffuse corneal opacification that makes the entire cornea appear hazy. Patient had PKP 18 years later. B: With use of retro illumination, a denser central opacity is apparent.

FIG. 49: External photograph of the cornea of a 49 year old male, II 5, from family B, with BCVA 20/30 and central and midperipheral corneal haze, central crystals and arcus lipoides. Arcus was prominent enough to see without the aid of a slit lamp. Patient subsequently had PKP for complaints of decreased vision and glare.

FIG. 50: Flow chart of SCCS patient survey and phone call follow up.

FIG. 51: Flow chart of change in visual acuity in SCCD patient with at least 7 years of follow up.

FIGS. 52A-B: A: External photograph of the cornea of a 33 year old male, patient number 1, in family Q, with BCVA 20/25, central subepithelial crystals and arcus lipoides (Photograph has been lightened to increase contrast and allow best visualization of crystal deposition). B: 8 years later, patient is 41 years old with BCVA 20/50 with increased central crystalline opacity, mid peripheral haze and arcus lipoides. PTK which was subsequently performed within the year did not increase BCVA and patient subsequently underwent PKP.

FIGS. 53A-F: Serial external photos of the eyes of a 39 year old woman, patient number 1, in family C, with amblyopia OS and BCVA of 20/30 OD and 20/400 OS demonstrating central corneal disc opacity, few inferior central subepithelial crystals, midperipheral haze and arcus lipoides. Increasing density of corneal haze is demonstrated over 17 year follow up. BCVA at age 56 is 20/50 OD and 20/400 OS and PKP was planned. A: External photo of OD at age 39. B: External photo of OS at age 39. C: External photo of OD at age 52. D: External photo of OS at age 52. E: External photo of OD at age 56. F: External photo of OS at age 56.

FIG. 54: SCCD PKP flow chart for age at first PKP.

FIG. 55: Age versus corneal surgery prevalence in SCCD left Y axis represents number of patients, right Y axis represent percentage of patients. X axis represents decade of age in years (yrs.) on most recent contact. Blue columns represent total number of patients in each decade of age. Red columns represent number of patients reporting prior corneal surgery on the most recent contact. Red line indicates percentage of patients in each decade of age with history of corneal surgery.

FIG. 56: Flow chart of cholesterol measurements in patients undergoing PKP/PTK.

FIG. 57: Diagram of corneal changes with age from Weiss, Cornea 1992; 11:93-101.

FIG. 58: External photograph of eyes of 68 year old female, I 1, from family B, with clear cornea after PKP OD and “cloudy” cornea OS from SCCD. Bilateral arcus lipoides is apparent.

FIG. 59: External photograph of cornea of an 80 year old male, 12, in family J, with BCVA of 20/30 OD and diffuse corneal haze with tessellations reminiscent of central cloudy dystrophy of Francois or posterior crocodile shagreen. OS had undergone PKP 3 years before.

FIG. 60: 53 year old male, II 1, in family J (son of patient I 1 in FIG. 35) with BCVA 20/25 OU, central corneal haze and crystals, mid peripheral haze and arcus lipoides.

FIG. 61: Light microscopy of the SCCD cornea with reddish hue from staining of the lipid deposits with oil red O (oil red O×40).

FIG. 62: Fluorescence noted from stromal deposition of filipin stained lipid (filipin x40).

FIGS. 63A-B: A: Basal epithelial cells, corneal stroma and few endothelial cells demonstrated dissolved lipid and cholesterol (toluidine blue, ×250). B: Electron microscopy demonstrating lipid deposits in posterior stroma and pre-Descemet's area. (x9900).

### DETAILED DESCRIPTION OF THE DISCLOSURE

Schnyder's crystalline corneal dystrophy (SCCD; OMIM 121800) is a rare autosomal dominant ocular disease characterized by an abnormal increase of cholesterol and phospholipid deposition in the cornea leading to progressive corneal opacification. Although SCCD was previously mapped to a genetic interval between markers D1S1160 and D1S1635, information reclassifying a previously unaffected individual expanded the interval to D1S2667 and included 9 additional genes. Three candidate genes which may be involved in lipid metabolism and/or are expressed in the cornea: UbiA prenyl-transferase Domain containing 1 (UBIAD1), FRAP1 and ANGPTL7 were analyzed.

Understanding the gene function leads to a further understanding of lipid metabolism. For example, Gaynor, et al., Arterioscler Thromb Vasc Biol 1996; 16(8):993-9, discloses accumulation of high-density lipoprotein (HDL) apolipoproteins in SCCD. This has implications for abnormal cholesterol accumulation in other conditions, such as atherosclerosis, and detection of mutations such as those described herein can provide new methods of screening for atherosclerosis, and for future vision loss and/or future need for corneal transplant.

DNA samples were obtained from three families with clinically confirmed SCCD. Analysis of FRAP1, ANGPTL7 and UBIAD1 was carried out using PCR-based DNA sequencing to examine protein coding regions, RNA splice junctions, and 5′ UTR exons. No disease-causing mutations were found in the FRAP1 or ANGPTL7 genes. Three non-synonymous mutations in conserved amino acids of UBIAD1 were identified in all three families with SCCD. The mutations are expected to interfere with the function of the UBIAD1 protein because they are located in highly-conserved and structurally important domains. Predictions of the protein structure indicated that a prenyltransferase domain and several transmembrane helices are affected by these mutations. Each mutation cosegregated with the disease in the families. Mutations were not observed in 95 normal DNA samples (190 chromosomes).

Having now generally described the present disclosure, the same will be more readily understood through reference to the following examples which are provided by way of illustration, and are not intended to be limiting of the present disclosure.

### I. Example 1

### A. Methods

### 1. Sample Collection

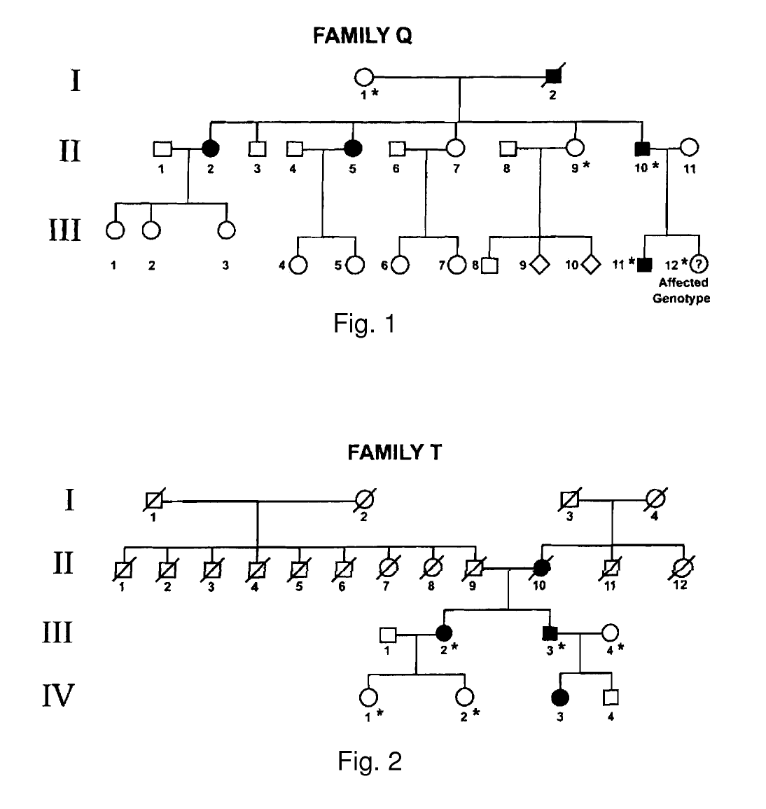

The recruitment efforts which spanned from 1987 to the present have been described in prior publications (Shearman, et al., Hum Mol Genet. 1996; 5:1667-1672, Theendakara, et al., Hum Genet. 2004; 114:594-600) with Institutional Review Board approval of the study obtained from University of Massachusetts Medical Center from 1992-1995 and subsequently from Wayne State University to the present. Written informed consent was obtained from all adult participants and the parent of minor participants under research tenets of the Declaration of Helsinki. Opthalmologic examination included assessment of visual acuity and performance of slit-lamp examination to assess corneal findings. Blood samples were collected from three unrelated SCCD pedigrees (FIGS. 1, 2 and 3). Genotyping of two of these families has been previously published. Families Q and Y were called pedigree 11 and 12, respectively, in the article by Theendakara et al., (Hum Genet. 2004; 114:594-600). Genotyping of Family T had not been previously reported.

### 2. DNA Isolation and PCR

Genomic DNA was isolated using the PUREGENE® DNA isolation kit (GentraSystems, Minneapolis, Minn.). DNA samples were quantified using the NanoDrop® ND-1000 Spectrophotometer (Thermo Scientific, Wilmington, Del.) and then diluted to approximately 20 ng/μl working solutions.

PCR products were designed to amplify exons and RNA splice junctions. Amplification of DNA was carried out in 25 μl reactions using 50 ng of genomic DNA and Hot-Start Taq DNA polymerase (Denville Scientific, Metuchen, N.J.) with 1× reaction buffer, 0.2 mM of each dNTP, and 0.2 μM each of forward and reverse primer. Thermal cycling was accomplished using MJ Research (Bio-Rad, Waltham, Mass.) Dyad and Tetrad DNA Engines and a program of 95° C. for 2 min, 10 cycles of touchdown PCR, and then 30 cycles of 95° C. for 30 s, 58° C. for 30 s, and 68° C. for 30 s; followed by a final 5 min extension at 68° C. PCR products (5 μl) were analyzed on 2% agarose gels and visualized with ethidium bromide.

### 3. DNA Sequencing

In some cases prior to sequencing, excess PCR primers were removed from 10 μA PCR product using Ampure PCR Purification (Agencourt Bioscience, Beverly, Mass.). Purified product was eluted in 30 μA of de-ionized water. Reaction chemistry using BigDye v. 3.1 (Applied Biosystems, Foster City, Calif.) and cycle sequencing were adapted from the manufacturer's recommendations. Cycle sequencing products were purified using CleanSeq reagents (Agencourt Bioscience Corp., Beverly, Mass.). Purified sequencing products were eluted in 40 μl of 0.01 μM EDTA and 30 μA was run on an ABI 3100 Genetic Analyzer. Sequence chromatograms were analyzed by Sequencher software (GeneCodes, Ann Arbor, Mich.) to visualize and align sequence chromatograms, as well as by Mutation Discovery (www.mutationdiscovery.com). The UCSC genome browser (www.genome.ucsc.edu) was used for protein and single nucleotide polymorphisms (SNP) annotation.

### B. Results

All protein coding regions, RNA splice junctions, and 5′ untranslated region (UTR) exons were examined from FRAP1, ANGPTL7 and UBIAD1 genes. Sequence variants were found in the FRAP1 and ANGPTL7 genes, but they were either present in both affected and unaffected individuals or they had been previously identified and were annotated in the SNP database (dbSNP, data not shown). In UBIAD1, DNA sequencing revealed mutations in affected members from all three families examined (Table 1).

TABLE 1
Mutations Identified in Three SCCD Families

| Family and | | |
|---|---|---|
| Individual ID | Mutation | Codon |
| T III-3 | GGT > CGT | G177R |
| Q II-11 | AAC > AGC | N102S |
| Y II-3 | AAC > AGC | N102S |

In Family Q (FIG. 1), two affected and two unaffected individuals were sequenced and both of the affecteds (II-10 and III-11) shared the N102S mutation, whereas the unaffecteds (1-1 and 11-9) did not have this mutation. Both affecteds had evidence of corneal crystal deposition on slit-lamp examination. The clinical status of III-12, a 19-year-old female, who was previously classified as unaffected (Theendakara, et al., Hum Genet. 2004; 114:594-600), was not clear. The examiner was unsure whether this patient might have a slight corneal haze suggestive of early SCCD without crystals. Sequencing revealed that she had an allele with the N102S mutation in two independent DNA samples reducing the likelihood of sample mislabeling or other technical errors. Reconstruction of haplotypes from the published data with the correct classification permits a disease haplotype to be shared by all three affected individuals (Theendakara, et al., Hum Genet. 2004; 114:594-600).

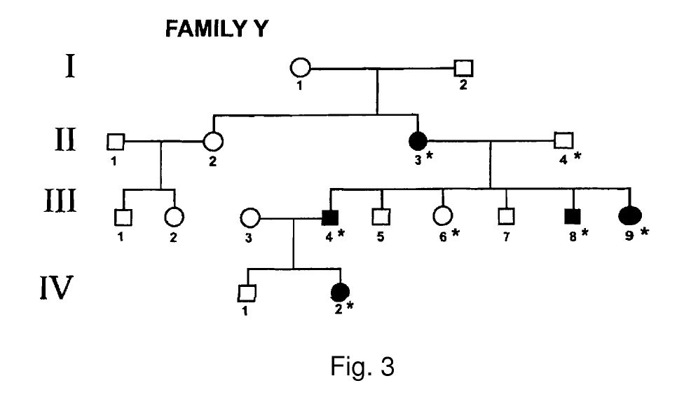

Family T (FIG. 2) was found to have a G177R mutation in both affected siblings (III-2 and III-3) available for the study and neither of the two unaffected children (IV-1 and IV-2) of individual III-2. An unaffected spouse (III-4) also did not have the mutation. The third SCCD family, Family Y (FIG. 3) had the same mutation as Family Q in all five affecteds available for the study. The one unaffected sibling (III-6) and her unaffected mother (II-4), whose DNA was also sequenced, did not have the mutation. In summary, all of the nine definitively affected individuals analyzed in the three families had a mutation and none of the six unaffected blood relatives had the mutation. The only exception was one individual who had the mutation, but whose clinical phenotype was not clear. Each mutation, therefore, cosegregated with the disease and was not seen in any of those family members who were definitively diagnosed on slit-lamp examination as unaffected.

Furthermore, the UBIAD1 gene was sequenced in 95 normal Caucasian samples and none of them were found to have any of the mutations.

Both mutations changed conserved bases that caused substitutions of amino acids conserved in 11 of 12 vertebrate species ranging from telostomes to man. The nonconservation for N102S was in the platypus, which had an isoleucine at amino acid 102, and for G177R it was in the armadillo, which had a two amino acids deleted. This evolutionary conservation potentially indicates key roles for these amino acids in normal function of the protein.

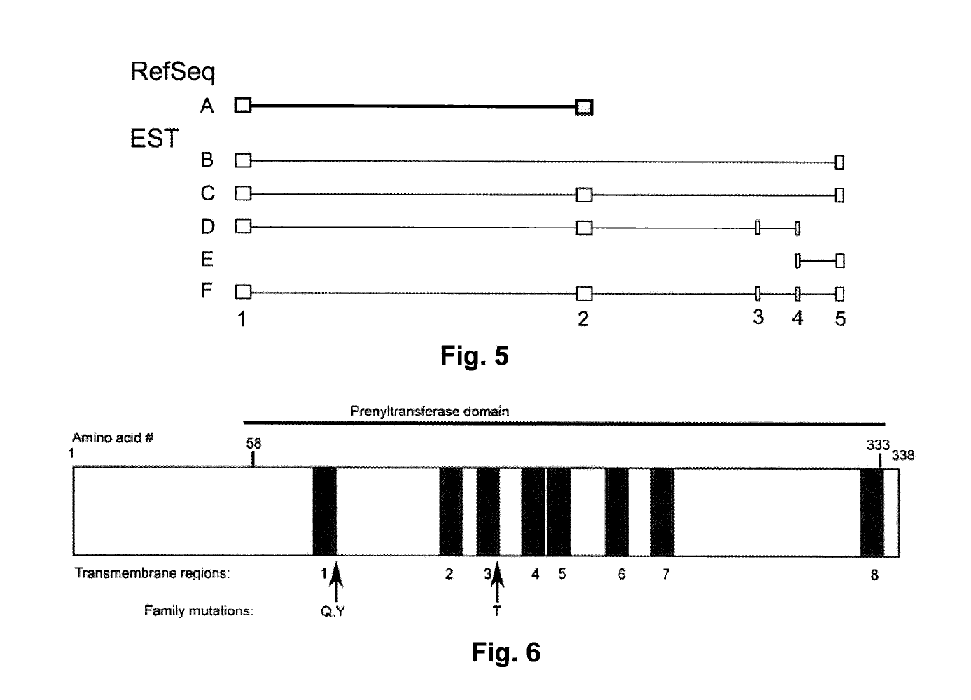

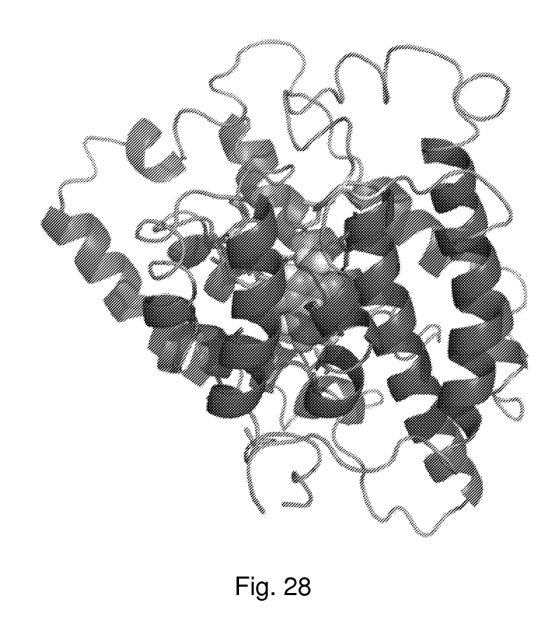

The UBIAD1 locus produces five transcripts that share exon 1, but exons 2 through 5 are transcript-specific. Also, transcripts A, C, D, and F, share exons 1 and 2, which comprise the curated UBIAD1 transcript (RefSeq NM_{—}013319; FIG. 5). The predicted protein structure for transcript A is shown in FIG. 28.

### C. Discussion

### 1. Difficulty of Making the Diagnosis

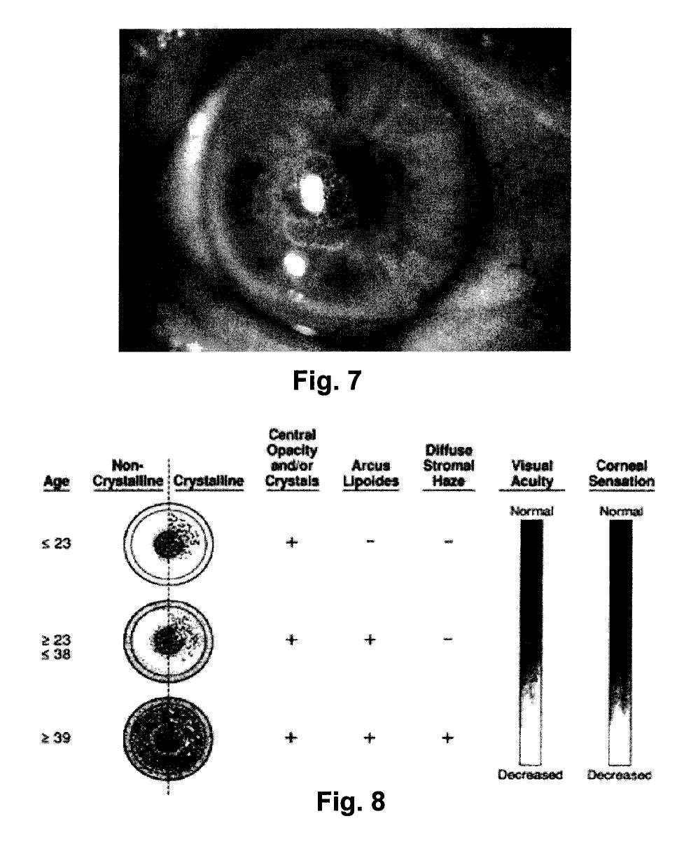

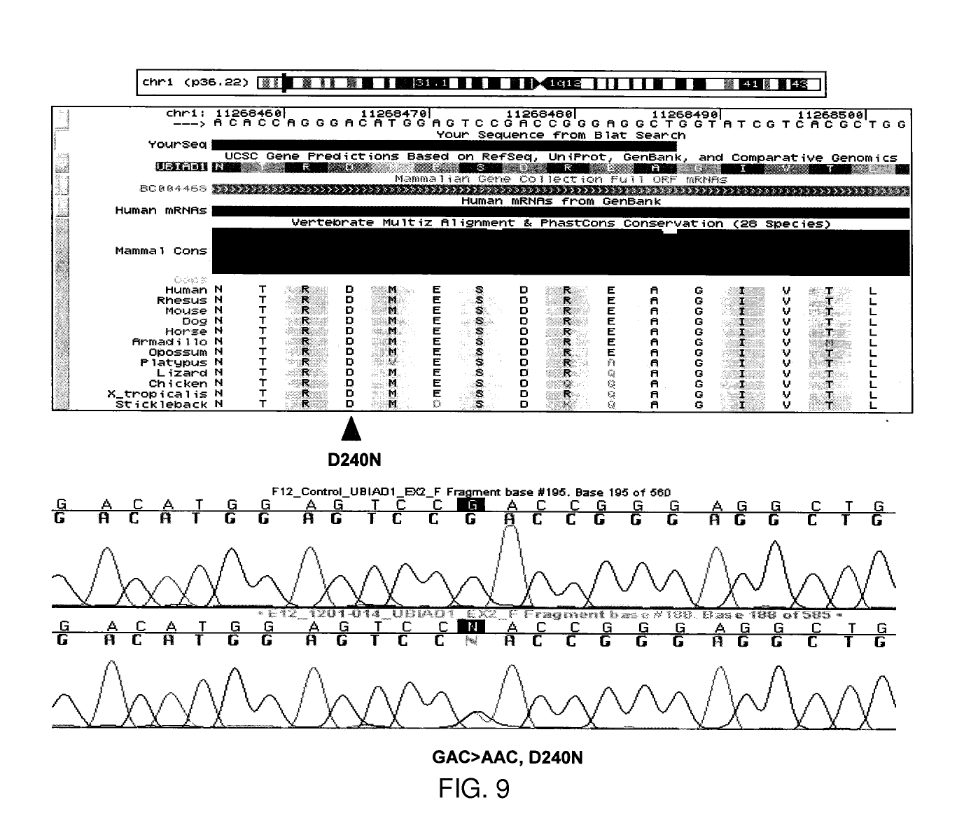

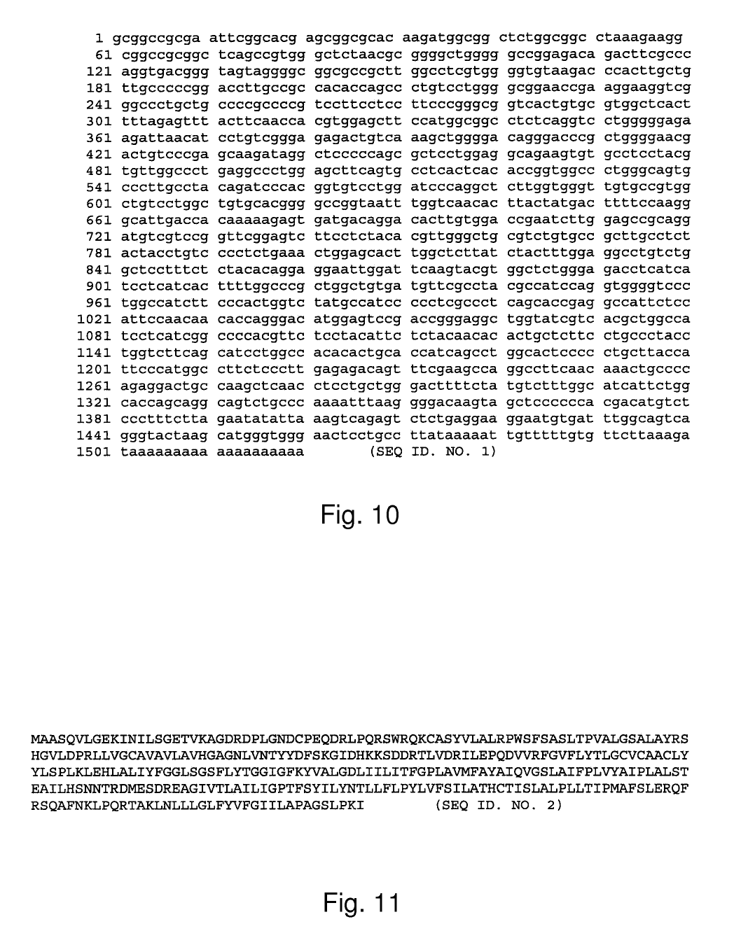

While most authors have described the clinical appearance of SCCD to include the presence of anterior corneal crystals, clinical examination of the four Swede-Finn pedigrees demonstrated that only 50% (9/18) of affected patients had corneal crystals (Weiss, Cornea 1992; 11:93-101; Weiss, Opthalmology 1996; 103:465-473; Weiss, Trans Am Opthalmol Soc 2007; 105:616-648). This percentage is confirmed by more recent clinical data from long term follow up of 33 SCCD pedigrees (Weiss, Cornea 1992; 11:93-101; Weiss, Opthalmology 1996; 103:465-473; Weiss, Trans Am Opthalmol Soc 2007; 105:616-648), in which one of the authors (Weiss) reported that on slit-lamp examination of SCCD patients, only 57% of eyes (48 of 87) had corneal crystalline deposits. In addition, the pattern of progressive corneal opacification was predictable based on age, regardless of the presence or absence of crystalline deposition. (Weiss, Cornea 1992; 11:93-101) (FIG. 7) However, because it is challenging to make the diagnosis of SCCD in the absence of crystals (Weiss, Opthalmology 1996; 103:465-473), Weiss proposed the alternative name, Schnyder's crystalline dystrophy sine crystals (SCCD sine crystals). While SCCD with crystals can be diagnosed as early as 17 months of age, diagnosis of SCCD without crystals can be delayed to the fourth decade because it is difficult to determine when the cornea demonstrates the first changes of subtle panstromal haze. Consequently, the assignment of an unaffected phenotype is more challenging in younger patients and might explain the findings in the 19 year-old female patient (III-12 in pedigree 11) who was previously classified as clinically unaffected (Theendakara, et al., Hum Genet. 2004; 114:594-600), yet carries a newly constructed disease haplotype and the mutation (N1025), also found in her affected brother, father and two paternal aunts. The alternative explanation is incomplete penetrance, a common phenomenon.

### 2. Corneal Lipid Deposition in SCCD

Although some suggest that the course of SCCD is benign with “visual acuity often unaffected” (Ingraham, et al., Opthalmology 1993; 100:1824-1827) and that SCCD rarely requires corneal grafting (Weller, et al., Br J Opthalmol 1980; 64:46-52); long term follow up of 33 of the pedigrees followed by Weiss up to 18 years revealed that 54% of patients (20 of 37) with SCCD who were 50 years of age and older had undergone penetrating keratoplasty (PKP) surgery. (Weiss, Trans Am Opthalmol Soc 2007; 105:616-648).

Patients with SCCD have been found to develop corneal arcus by 23 years of age. (Weiss, Cornea 1992; 11:93-101) While premature occurrence of corneal arcus is reported to be associated with coronary artery disease, (Halfon, et al., Br J Opthalmol 1984; 68:603-604; Rouhiainen, et al., Cornea 1993; 12:142-145; Virchow, Virchow's Arch Path Anat 1852; 4:261-372), corneal arcus can occur independently of abnormal lipid levels or other systemic disorders. (Barchiesi, et al., Sury Opthalmol 1991; 36:1-22). Hypercholesterolemia is present in up to 2/3 of patients with SCCD. (Kajinami, et al., Nippon Naika Gakkai Zasshi 1988; 77:1017-1020; Karseras, et al., Br J Opthalmol 1970; 54:659-662; Williams, et al., Trans Opthalmol Soc UK 1971; 91:531-541). Although familial hypertriglyceridemia and dysbetalipoproteinemia have been reported, familial hypercholesterolemia is the most common lipoprotein abnormality found in patients with SCCD. (Crispin, Prog Retin Eye Res 2002; 21:169-224). These abnormalities can also occur in members of the SCCD pedigrees who are reported to be unaffected by the corneal dystrophy. (Barchiesi, et al., Sury Opthalmol 1991; 36:1-22; Bron, et al., Br J Opthalmol 1972; 56:383-399; Kajinami, et al., Nippon Naika Gakkai Zasshi 1988; 77:1017-1020; Yamada, et al., Br J Opthalmol 1998; 82:444-447). By comparison, the Cavalier King Charles Spaniel and rough collie breeds of dog with crystalline dystrophy usually have normal serum lipid levels. (Crispin, et al., Clin Sci 1988; 74:12).

Previously, the systemic hyperlipidemia in SCCD was postulated to be the primary defect which resulted in corneal clouding but this theory lost favor when others documented that patients affected with SCCD can have either normal or abnormal serum lipid, lipoprotein or cholesterol levels and that the progress of the corneal opacification is not related to the serum lipid levels. (Sysi, Br J Opthalmol 1950; 34:369-374; Lisch, et al., Ophthalmic Paediatr Genet. 1986; 7:45-56). Lisch followed 13 patients with SCCD for 9 years and concluded that no link could be drawn between the corneal findings and systemic hyperlipidemia although 8 of 12 patients had elevated cholesterol or apolipoprotein B levels and 6/8 had dyslipoproteinemia type IIa. (Lisch, et al., Ophthalmic Paediatr Genet. 1986; 7:45-56).

Histopathologic examination of SCCD corneal specimens demonstrates abnormal lipid deposition throughout the corneal stroma, (McGarvey, et al., J Cell Biochem 2005; 95:419-428; Peek, et al., Investigative Opthalmology & Visual Science 1998; 39:1782-1788; Hoang-Xuan, et al., J Fr Ophtalmol 1985; 8:735-742; Kaden, et al., Albrecht Von Graefes Arch Klin Exp Opthalmol 1976; 198:129-138; Thiel, et al., Klin Monatsbl Augenheilkd 1977; 171:678-684; Weller, et al., Br J Opthalmol 1980; 64:46-52; Delleman, et al., Opthalmologica 1968; 155:409-426; Ingraham, et al., Opthalmology 1993; 100:1824-1827; Halfon, et al., Br J Opthalmol 1984; 68:603-604; Rouhiainen, et al., Cornea 1993; 12:142-145; Virchow, Virchow's Arch Path Anat 1852; 4:261-372; Barchiesi, et al., Sury Opthalmol 1991; 36:1-22; Karseras, et al., Br J Opthalmol 1970; 54:659-662) basal epithelium and occasionally within the endothelial cells with the crystalline deposits which occur in some patients having been shown to be cholesterol. (Garner, et al., Br J Opthalmol 1972; 56:400-408; Delleman, et al., Opthalmologica 1968; 155:409-426: Bonnet, et al., Bull Soc Ophtalmol Fr 1934; 46:225-229; Rodrigues, et al., Am J Opthalmol 1990; 110:513-517). Lipid analysis demonstrates excess accumulation of unesterified cholesterol, esterified cholesterol, and phospholipid. (Weiss, et al., Opthalmology 1992; 99:1072-1081)

It has been proposed that the gene for SCCD resulted in an imbalance in local factors affecting lipid/cholesterol transport or metabolism. A temperature-dependent enzyme defect had been postulated because the initial cholesterol deposition occurs in the axial/paraxial cornea, which is the coolest part of the cornea (Crispin, Prog Retin Eye Res 2002; 21:169-224; Burns, et al., Trans Am Opthalmol Soc 1978; 76:184-196). The cornea as an active uptake and storage site for cholesterol has been documented. Radiolabeled 14-C cholesterol was injected 11 days prior to removing a patient's cornea during PKP and demonstrated the level of radiolabeled cholesterol was higher in the cornea than the serum at the time of surgery. (Burns, et al., Trans Am Opthalmol Soc 1978; 76:184-196). Furthermore, lipid analysis of the corneal specimens from patients affected with SCCD who have undergone PKP revealed that the apolipoprotein constituents of HDL (apo A-I, A-II and E) were accumulated in the central cornea while those of low-density lipoprotein (LDL) (apo B) were absent. This suggested an abnormality confined to HDL metabolism. (Gaynor, et al., Arterioscler Thromb Vasc Biol 1996; 16:992-999).

Because of its smaller size, HDL would be the only lipoprotein that could freely diffuse, while intact, to the central cornea. The size of the larger lipoproteins would prevent their free diffusion unless they were modified. (Bron, Cornea 1989; 8:135-140). HDL concentrations are inversely related to the incidence of coronary atherosclerosis (Mayes, Harpers Biochemistry: Cholesterol Synthesis, Transport and Excretion 2005; 26). Consequently, SCCD lipid accumulation could be caused by a local defect of HDL metabolism. Alternatively, because HDL-related apolipoproteins tend to associate with lipid, the accumulation of these apolipoproteins in the cornea could be secondary to lipid that accumulates in the cornea for some other reason.

The possibility that the gene for SCCD plays an important role in lipid/lipoprotein metabolism throughout the body is supported by an article by Battisti and coworkers (Battisti, et al., Am J Med Genet. 1998; 75:35-39) who cultured the skin fibroblasts of a patient with SCCD. Although membrane bound spherical vacuoles with lipid materials suggesting storage lipids were present in the skin, this work has not been repeated in the literature.

### 3. UBIAD1 and Lipid Metabolism

UBIAD1 is of interest as this gene produces a protein that contains several transmembrane domains and a prenyltransferase domain that potentially could play a role in cholesterol metabolism. UBIAD1, UbiA prenyltransferase domain containing 1, is also known as TERE1, or RP4-796F18. The TERE 1 transcript is present in most normal human tissue including cornea. It has been demonstrated that the expression of this gene was greatly decreased in prostate carcinoma. UBIAD1 interacts with the C terminal portion of apolipoprotein E14, which is known to be important in reverse cholesterol transport because it helps mediate cholesterol solubilzation and removal from cells. Apolipoprotein E was found to be present at increased levels in corneal specimens from SCCD corneas. Consequently, a potential mechanism for UBIAD1 gene-mediated cornea lipid cholesterol accumulation is that its interaction with apolipoprotein E, and possibly other HDL lipid solubilizing apolipoproteins, in the cornea, results in decreased cholesterol removal from the cornea.

There is another possible mechanism by which a mutated UBIAD1 gene could result in corneal cholesterol accumulation. This gene contains a prenyltransferase domain suggesting that the gene functions in cholesterol synthesis. Prenylation reactions are involved in cholesterol synthesis as well as the synthesis of geranylgeraniol, an inhibitor of HMG-CoA reductase, the rate limiting enzyme in cholesterol synthesis. Thus, it is possible that the UBIAD1 functions in regulating cholesterol synthesis and that excess cholesterol synthesis occurs when this gene is defective. In this regard, increased cholesterol synthesis in the liver and other tissues would be expected to downregulate the LDL receptor that mediates removal of LDL from the blood, thus accounting for the elevated LDL blood levels often observed in SCCD patients.

The potential consequences of the mutations described in this study on UBIAD1 protein function need to be investigated. Additionally, the UBIAD1 locus produces five transcripts that share exon 1, but exons 2 through 5 are transcript-specific. An expanded mutation spectrum can help identify which transcript produces the protein that, when mutated, causes SCCD. Furthermore, an expanded spectrum of mutations can assist in identification of genotype-phenotype correlations that highlight specific functions of the protein that, when mutated, lead to family-specific SCCD characteristics.

### II. Example 2

### Visual Morbidity in Thirty Three Families with SCCD

Example 2 was performed to assess the findings, visual morbidity, and surgical intervention in SCCD.

### A. Summary

### 1. Methods

There have been 115 retrospective case series of affected individuals from 34 SCCD families identified since 1989. Age, uncorrected visual acuity, best-corrected visual acuity (BCVA), corneal findings, and ocular surgery were recorded. Prospective phone, e-mail, or written contact provided updated information. Patients were divided into 3 age categories for statistical analysis: less than 26 years of age, 26 to 39 years of age, and 40 years of age and older.

### 2. Results

Mean age on initial examination was 38.8±20.4 (range, 2-81) with follow-up of 55 of 79 (70%) of American patients. While there were no statistically significant correlations between logMAR visual acuity and age (logMAR BCVA=0.033+0.002×age; R=0.21), the linear regression showed the trend of worse visual acuity with age. BCVA at ≧40 years was decreased compared to <40 (P<0.0001), although mean BCVA was >20/30 in both groups. Twenty-nine of 115 patients had corneal surgery with 5 phototherapeutic keratectomy (PTK) (3 patients), and 39 PKP (27 patients). PKP was reported in 20 of 37 (54%) patients ≧50 years and 10 of 13 (77%) of patients ≧70. BCVA 1 year prior to PKP in 15 eyes (9 patients) ranged from 20/25 to 20/400 including 7 eyes with other ocular pathology. BCVA in the remaining 8 eyes was 20/25 to 20/70 with 3 of these 4 patients reporting preoperative glare. Chart and phone survey suggested increasing difficulty with photopic vision with aging.

### 3. Conclusion

Although excellent scotopic vision continues until middle age in SCCD, most patients had PKP by the 7th decade. SCCD causes progressive corneal opacification, which can result in glare and disproportionate loss of photopic vision.

### 4. Systemic Lipid Abnormalities

The incidence of hypercholesterolemia in SCCD has been reported to be up to 66% of affected patients. (Bron, Cornea 1989; 8:135-140; Brownstein, et al., Can J Opthalmol 1991; 26:273-279; Sverak, et al., Cesk Oftalmol 1969; 25:283-287). Although many patients with SCCD have hypercholesterolemia, most investigators agree that the severity of the dyslipidemia is not correlated to the occurrence of crystalline formation (McCarthy, et al., Opthalmology 1994; 101:895-901) and that the progress of the corneal opacification is not related to the serum lipid levels (Lisch, et al., Ophthalmic Paediatr Genet. 1986; 7:45-56; Sysi, Br J Opthalmol 1950; 34:369-374). Patients affected by the corneal dystrophy can have normal or abnormal serum lipid, lipoprotein, or cholesterol levels. Likewise, serum lipid, lipoprotein, or cholesterol levels can be normal or abnormal in members of the pedigree without the corneal dystrophy. (Bron, Cornea. 1989; 8:135-140; Barchesi, et al., Sury Opthalmol 1991; 36:1-22; Bron, et al., Br J Opthalmol 1972; 56:383-399; Kajinami, et al., Nippon Naika Gakkai Zasshi 1988; 77:1017-1020; Yamada, et al., Br J Opthalmol 1998; 82:444-447).

### B. Corneal Findings and Confusion in the Published Literature

1. Corneal Crystals and SCCD

Most investigators have described the clinical appearance of SCCD to include the bilateral deposition of anterior stromal crystals early in life with subsequent appearance of corneal arcus and stromal haze (Lisch, et al., Ophthalmic Paediatr Genet. 1986; 7:45-56; Sysi, Br J Opthalmol 1950; 34:369-374; Bron, et al., Br J Opthalmol 1972; 56:383-399; van Went, et al., Niederl Tijdschr Geneesks 1924; 68:2996-2997; Schnyder, Schweiz Med Wochenschr 1929; 10:559-571; Bec, et al., Bull Soc Ophtalmol Fr 1979; 79:1005-1007; Chem, et al., Am J Opthalmol 1995; 120:802-803; Delogu, Ann Ottalmol Clin Ocul 1967; 93:1219-1225; DiFerdinando, G Ital Oftalmol 1954; 7:476-484; Freddo, et al., Cornea. 1989; 8:170-177; Garner, et al., Br J Opthalmol 1972; 56:400-408; Grop, Acta Opthalmol Suppl (Copenh) 1973; 12:52-57; Hoang-Xuan, et al., J Fr Ophtalmol 1985; 8:735-742; Kaden, et al., Albrecht Von Graefes Arch Klin Exp Opthalmol 1976; 198:129-138; Lisch, Klin Monatsbl Augenheilkd 1977; 171:684-704; Mielke, et al., Opthalmologe 2003; 100:158-159; Rodrigues, et al., Am J Opthalmol 1987; 104:157-163; Thiel, et al., Klin Monatsbl Augenheilkd 1977; 171:678-684; Weller, et al., Br J Opthalmol 1980; 64:46-52; Delleman, et al., Opthalmologica 1968; 155:409-426) typically suggesting that the finding of cholesterol crystals is integral to the diagnosis.

However, SCCD in the absence of corneal crystal deposition has also been described. (Lisch, et al., Ophthalmic Paediatr Genet. 1986; 7:45-56; Bron, et al., Br J Opthalmol 1972; 56:383-399; Grop, Acta Opthalmol Suppl (Copenh) 1973; 12:52-57; Lisch, Klin Monatsbl Augenheilkd 1977; 171:684-704; Delleman, et al., Opthalmologica 1968; 155:409-426; Weiss, et al., Opthalmology 1992; 99:1072-1081). In fact, a report of 4 Swede-Finn pedigrees with 18 affected members revealed that only 50% of patients actually had corneal crystals. (Weiss, Cornea. 1992; 11:93-101). Examination of these patients demonstrated that the characteristic corneal change of SCCD was a progressive diffuse opacification of the cornea.

Despite published documentation about the varied spectrum of corneal changes in this dystrophy, more recent publications continue to emphasize the importance of crystals in the diagnosis of SCCD, reporting that “the clinical appearance of this dystrophy varies, but it is characterized by the bilateral and usually symmetric deposition of fine, needle-shaped polychromatic cholesterol crystals”. (Paparo, et al., Cornea 2000; 19:343-347). The presumption that most, if not all, SCCD patients have corneal crystals can increase the difficulty of making the diagnosis of SCCD in the patient who has findings typical of SCCD but does not have crystalline deposits.

2. Clinical Course

Although SCCD is a progressive disease, (Grop, Acta Opthalmol Suppl (Copenh) 1973; 12:52-57) as recently as the last decade one investigator wrote that the disease “is often described as stationary” (Kohnen, et al., Klin Monatsbl Augenheilkd 1997; 211:135-136) and another indicated that the disease classically was “non-progressive . . . however, rare sporadic cases and individuals with progressive, panstromal Schnyder dystrophy have been described.” (Ingraham, et al., Opthalmology 1993; 100:1824-1827). It is possible that the rarity of the dystrophy compounded by the confusion about clinical findings, has previously resulted in surgical biopsy of the SCCD cornea in order to assist the ophthalmologist in making the diagnosis (Brownstein, et al., Can J Opthalmol 1991; 26:273-279; Ingraham, et al., Opthalmology 1993; 100:1824-1827). In fact, as recently as 2001, one published report indicated that the diagnosis of the disease was based on “clinical findings and corneal biopsy.” (Ciancaglini, et al., J Cataract Refract Surg 2001; 27:1892-1895).

3. PKP and PTK

Most articles have suggested that the course of the dystrophy is typically benign with some indicating that “visual acuity [is] often unaffected.” (Ingraham, et al., Opthalmology 1993; 100:1824-1827). Although there are frequent reports of PKP in SCCD, (Lisch, et al., Ophthalmic Paediatr Genet. 1986; 7:45-56; Yamada, et al., Br J Opthalmol 1998; 82:444-447; Freddo, et al., Cornea 1989; 8:170-177; Hoang-Xuan, et al., J Fr Ophtalmol 1985; 8:735-742; Rodrigues, et al., Am J Opthalmol 1987; 104:157-163; Weller, et al., Br J Opthalmol 1980; 64:46-52; Delleman, et al., Opthalmologica 1968; 155:409-426) the literature has reported that SCCD rarely requires corneal grafting. (Weller, et al., Br J Opthalmol 1980; 64:46-52). With the advent of the excimer laser, PTK has been successful in removal of subepithelial crystals and improving symptoms of glare and photophobia associated with the corneal opacity (Paparo, et al., Cornea 2000; 19:343-347; Ciancaglini, et al., J Cataract Refract Surg 2001; 27:1892-1895; Dinh R, et al., Opthalmology 1999; 106:1490-1497; Fagerholm, Acta Opthalmol Scand 2003; 81:19-32; Herring, et al., J Refract Surg 1999; 15:489). Recurrence of the dystrophy after both PKP (Brownstein, et al., Can J Opthalmol 1991; 26:273-279; Lisch, et al., Ophthalmic Paediatr Genet. 1986; 7:45-56; Garner, et al., Br J Opthalmol 1972; 56:400-408; Delleman, et al., Opthalmologica 1968; 155:409-426) and PTK (Vesaluoma, et al., Opthalmology 1999; 106:944-951) has been reported.

4. Questions About SCCD Not Yet Answered

Although Lisch and associates, (Lisch, et al., Ophthalmic Paediatr Genet. 1986; 7:45-56) in 1986, reported a 9-year follow-up of 13 patients with SCCD, there have been no recent studies documenting the actual course of visual decrease with age in a large number of patients with SCCD. The frequency of corneal surgical intervention in SCCD has never been reported. The rarity of the dystrophy has dictated that most publications have been case reports or small series that describe visual acuity in a limited number of affected patients.

5. Four Large Swede-Finn Pedigrees With SCCD

In 1992, the results of clinical examinations of 18 patients affected with SCCD in 4 families from Massachusetts were published. (Weiss, Cornea 1992; 11:93-101). Each of the 4 pedigrees had Swede-Finn ethnicity. The histopathologic findings of corneal specimens obtained from PKP surgery were described. (Weiss, et al., Opthalmology. 1992; 99:1072-1081) Quantification of the corneal lipid was also reported (Gaynor, et al., Arterioscler Thromb Vasc Biol 1996; 16:993-999). Subsequently, the clinical findings of 33 members of these pedigrees were published (including the 18 original affected patients). (Weiss, Opthalmology 1996; 103:465-473).

6. Genetics: UBIAD1, The Causative Gene For SCCD

Since the initial article in 1992 to the present, the goal of isolating the genetic defect in the disease resulted in a continuation of recruitment efforts nationally and internationally to enroll additional patients with SCCD. Under Institutional Review Board (IRB) approval of the Human Investigations Committee of the University of Massachusetts Medical Center, specimens from the initial Swede-Finn families were used to map the disease to 1p36. (Shearman, et al., Hum Mol Genet. 1996; 5:1667-1672). With the identification of more families nationally and internationally, and using 13 families with SCCD, the genetic interval was further narrowed to 2.32 Mbp. Identity by state was present in all 13 families for two markers, which further narrowed the candidate region to 1.57 Mbp (Theendakara, et al., Hum Genet. 2004; 144:594-600). At the same time that specimens were collected for the genetic mapping studies, clinical information about the affected members of the SCCD pedigrees continued to be collected. On enrollment in the genetic mapping study, information about visual acuity, corneal examination, and history of corneal surgery was requested. Since 1989, a total of 36 families worldwide with SCCD have been identified with a total of 132 affected members. Using 6 of these pedigrees, the author and coworkers recently reported that mutations in the UBAID1 gene resulted in SCCD (Weiss, et al., Invest Opthalmol Vis Sci 2007; 48:5007-5012).

### C. Methods

The analysis of the clinical data in this large group of patients with SCCD represented an unusual opportunity to assess the visual impact of this disease. This study summarizes the clinical findings, visual acuity with age, and prevalence of corneal surgical intervention in the largest cohort of SCCD patients ever reported with the longest-term data yet reported in this disease. The recruitment and information gathering efforts for this study span 19 years from the recruitment of the first affected patients in 1987 to 2006. The recruitment methods varied during the 2 decades and are summarized below.

1. Initial Recruitment And Screening

a. History

Between July 1987 and October 1988, three unrelated individuals were referred for diagnosis of bilateral corneal opacities. Each patient was diagnosed as having SCCD. Interestingly, each of the three patients had a surname or maiden name of Johnson and had Swede-Finn ethnicity. Because this appeared to be a unique opportunity to study a large number of patients with this disease, a 3-part recruitment effort was begun in January 1989 (Weiss, Cornea 1992; 11:93-101).

Letters were sent to ophthalmologists in the community describing the corneal findings in SCCD and requesting that patients with these findings be referred for further evaluation. More than 600 letters were sent to patients in the local phone book with the name Johnson informing them of free ophthalmic screenings offered to identify patients with the dystrophy. In addition, articles publicizing free screenings were written for local newsletters, which were distributed in the Swede-Finn community.

Preliminary screening examinations performed from 1989 to 1995 included uncorrected visual acuity (UCVA) or best-corrected visual acuity (BCVA) and slit-lamp examination of the cornea. Patients noted to be unaffected on screening slit-lamp examination did not have complete ophthalmic examinations performed. Patients who were identified to have SCCD had dilated examination and corneal sensation testing. Testing of corneal sensation was performed before administration of eye drops by lightly touching the cornea with a wisp of cotton from a cotton swab or by performing Cochet Bonnet testing.

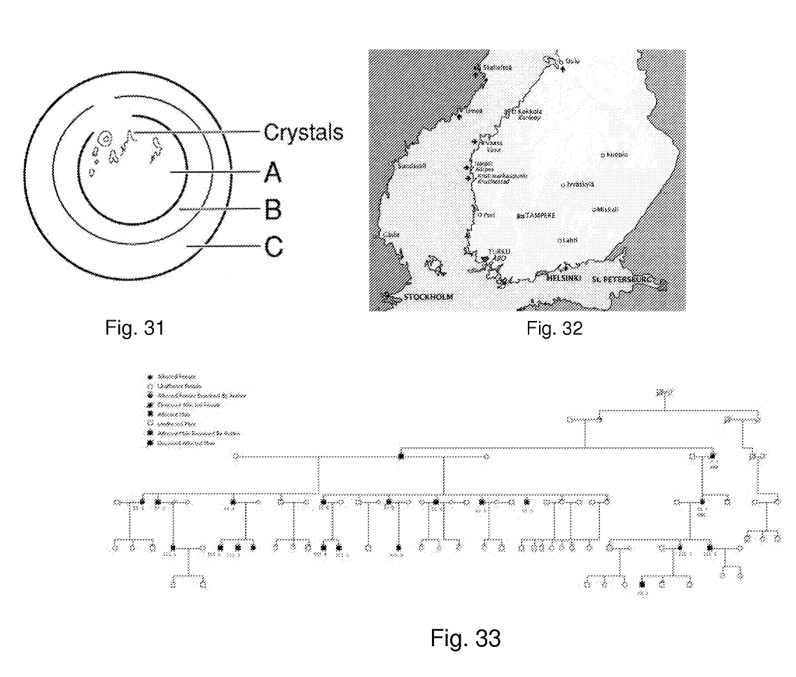

Notation was made of the location of specific corneal findings, including crystalline deposits, central disc opacity, midperipheral corneal haze, and arcus lipoides (FIG. 31). Selected patients had cholesterol analysis. (Weiss, Cornea 1992; 11:93-101). Patients were asked about family history, which allowed identification of other members of the family who could subsequently be examined. Gradually, individual pedigrees were established with indication of both the affected and unaffected individuals. The ancestors of the original four Swede-Finn pedigrees were found to originate from towns of Vasa, Narpes, and Kristinestad in a 60-km area on the west coast of Finland (FIG. 32). Aside from learning more about the corneal changes in SCCD, it appeared that examining large numbers of patients affected with SCCD could present an opportunity to isolate the genetic defect in the disease.

b. Present Study

Under IRB Approval of the University of Massachusetts Medical Center and the Wayne State University Medical School, different recruitment efforts were employed to attract additional SCCD patients to the study. Patients were recruited by referral from other physicians, referral from family members in affected pedigrees, or self-referral. Once an index patient agreed to participate in the study, the patient was asked to contact other family members to see if they would agree to be contacted. Throughout the years, additional pedigrees with SCCD were recruited for the study. The goal was to obtain clinical and genetic information from as many members of each SCCD pedigree as possible.

On the initial contact, patients were invited to complete a clinical data and family history form and/or submit a blood sample for genetic mapping. All studies were performed under the auspices of the IRB, and all patients who were willing to participate signed informed consent before participation.

Some patients, who were close enough geographically, underwent a complete eye examination with notation of BCVA, specific corneal findings noted on slit-lamp examination, dilated examination, and often corneal sensitivity testing. Notation was made if and when the patient had undergone corneal surgery, including PTK or PKP. Presence of genu valgum or history of prior surgery for genu valgum was indicated.

Other patients were requested to sign medical record releases so their examining ophthalmologist could be contacted for results of their examination. The ophthalmologist was asked to complete a 1-page sheet indicating the UCVA, BCVA, intraocular pressure, motility, complete slit-lamp examination with corneal findings on either eye including crystals, arcus, central disc opacities, and midperipheral haze, and other findings such as prior PKP and dilated examination. Corneal sensitivity testing was requested.

Enrolled patients were also requested to personally complete two forms. The first form was a one-page general health history, including general health questions and inquiries about hyperlipidemia and treatment. In addition, there was a nine-page family history questionnaire that asked names and ages of children, siblings, parents, grandparents, aunts and uncles, known health problems, and which members were thought to be affected with SCCD and when they were diagnosed. The family history was used to establish the individual SCCD pedigrees. Participants were also asked to provide contact information for other family members who expressed willingness to be contacted for the study.

Corneal sensation was checked with cotton swab or Cochet Bonnet when the patient had no prior ocular drops. Other physicians were asked to circle if testing was done with Q-tip, Cochet Bonnet or other. Any report of reduction in sensation by the examiner or a Cochet Bonnet measurement of 5/6 or less was recorded as decreased sensation.

2. Follow-Up Forms

To obtain long-term information on the enrolled patients, physicians of the referring foreign families were contacted by e-mail between 2005 and 2006 requesting updated clinical information.

Contact information was available on all of the families living in the United States from their initial study enrollment. In September 2005, using the original contact information, a medical record request form was sent to patients residing in the United States in order to obtain information about disease progression. Unfortunately, in the majority of cases, letters were either returned as undeliverable or patients did not respond. A record was made of those patients whose questionnaire was returned back stamped “return to sender” with the assumption that the patient had moved and there was no longer a forwarding address.

A list of corrected, current addresses for affected patients in the United States was established by using Internet search engines or by contacting known family members who could provide updated information for those family members whose address and phone numbers had changed.

a. Written Survey

American patients were mailed two separate questionnaires and a medical record release form. The eye history questionnaire was a three-page questionnaire including questions about other ocular diseases and details about any ocular surgery, including dates and type of surgery. Specific questions included whether the patient had one or more PKP procedures and, if so, the date, postoperative vision if known, and any problems experienced. Additional questions were directed at whether there were any affected family members who were now deceased, as well as a request for contact information for any previously unaffected members who were now diagnosed as having SCCD. Medical record request form for the ophthalmologist or optometrist and HIPAA (Health Insurance Portability and Accountability Act) information were included. Patient information was updated with results of the questionnaire as well as medical records that were received. Information about date and cause of death was included for SCCD patients who were reported to die during the course of the study. Patients who were newly affected with SCCD were mailed the eye history questionnaire and medical record release form.

The seven-page health history questionnaire asked patient's name; cholesterol, LDL, HDL and triglyceride measurement; and whether cholesterol-lowering medication was being taken. Additional questions included whether the patient or family members had diabetes, stroke, cerebrovascular accident, myocardial infarction, and hyperlipidemia; were taking lipid-lowering drugs; or had high blood pressure.

b. Telephone Survey

Telephone calls to clarify survey responses and to obtain information from those patients who did not answer the survey were made. Patients who had previously agreed to participate in the study were contacted by telephone to clarify answers supplied in written questionnaires that had been returned or to request that the questionnaire be completed and returned. In addition, during the phone call, patients were asked whether they or any affected family members had undergone PKP or other ocular surgery or had any ocular problems such as corneal graft rejection or dystrophy recurrence after PKP. Questions were also asked about systemic cholesterol and triglyceride levels, use of lipid-lowering agents, and past history of coronary artery disease, myocardial infarction, and cerebrovascular accident. Patients were also asked whether any family members had died and if so the age and cause of death. Information from patient telephone survey that was entered into the final data set included age and cause of death for deceased affected members, whether a patient had undergone PTK or PKP, and when and whether a patient was on a cholesterol lowering medication.

3. Data Recording

Information from the affected patient's initial examination was recorded, including family pedigree name, patient name, date of birth, date and age at first examination, name of the doctor who performed the examination, UCVA, BCVA, corneal findings including presence of crystals, central corneal haze, midperipheral corneal haze and/or arcus lipoides; whether dilated examination was performed; presence of cataract or other ocular pathology; history of ocular surgery, including PTK or PKP; and whether there was past or present history of genu valgum, which is known to sometimes be associated with the disease. (Hoang-Xuan, et al., J Fr Ophtalmol 1985; 8:735-742). If clinical photographs were available, these were also used to confirm or obtain information about corneal findings such as presence of corneal crystals, midperipheral haze, or arcus lipoides. If the information was not present or was unclear on chart or photograph review, the entry was listed as unknown. Symptoms or signs such as complaints of glare or results of glare testing, as well as use of lipid-lowering medication, were recorded if available from initial or follow-up examinations. Notation was made of any additional ocular pathology found on examination, such as amblyopia or cataracts. Patients with other ocular pathology or prior ocular surgery were eliminated from UCVA and BCVA analysis for initial examination and follow-up examinations.

BCVA included vision obtained with correction (glasses or contact lenses), with pinhole, or with manifest refraction. If all 3 were listed, the vision with manifest refraction was chosen. If the vision with glasses and vision with glasses and pinhole were available, the latter was chosen. UCVA and BCVA were converted to logMAR units for statistical analysis. Patients were divided into 3 age categories for statistical analysis: less than 26 years of age, 26 to 39 years of age, and 40 years of age and older.

When available, information obtained from serial ocular examinations from chart notes was recorded for the individual patients. This information allowed long-term follow-up of ocular findings in individual patients with SCCD. For those patients who underwent corneal surgery, preoperative UCVA or BCVA within 1 year of surgery was compared to UCVA or BCVA at the most recent visit. Patients who had at least 7 years between eye examinations were used to examine the changes in visual acuity over time.

To calculate the percentage of patients in each decade who had undergone corneal surgery, data from the most recent examination, telephone, or written contact was used. The patient's age, decade of age, and whether or not the patient reported having had PTK, photorefractive keratectomy (PRK), or PKP was recorded. The total number of patients in each decade was compared to the number of patients in that decade who had reported corneal surgery.

### D. Results

1. Demographics

Thirty-six families with SCCD were enrolled since 1987. Two pedigrees from Finland with 20 members had no clinical information and were initially excluded. Of the remaining 34 families, 13 families originated from outside the United States and 21 of the families were recruited from the United States (Table 1). Of these, 16 families were referred by other physicians, 4 families were self-referred because of SCCD, and one family presented directly to the author for routine clinical examination, at which time SCCD was diagnosed. In total, the author examined 8 of the 21 US pedigrees.

Of the 13 foreign pedigrees, 4 were from Germany, 3 were from Taiwan, 3 from England, 1 was from Turkey (Koksal, et al., Cornea 2004; 23:311-313), 1 from Japan, and 1 from Czechoslovakia. The author examined patients from 2 of the 3 Taiwanese pedigrees.

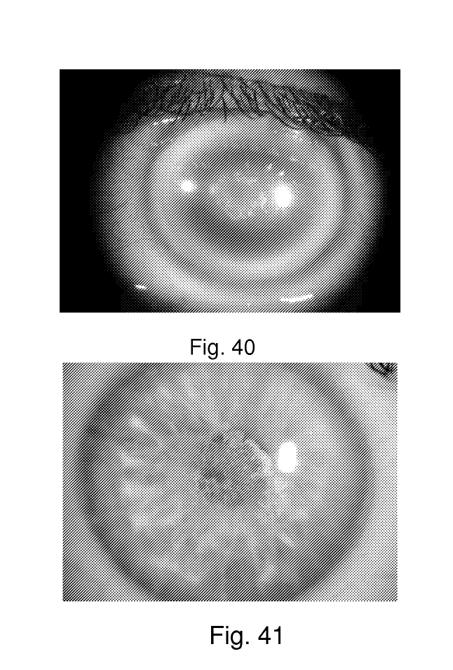

There were 115 affected patients in the 34 pedigrees. Of the 115 patients, 56 were female, 56 were male, and gender was not specified in 3 patients. Thirty of the pedigrees had 5 or fewer affected members in the family. The other 3 pedigrees were much larger: pedigree A had 19 affected members enrolled (FIG. 33), pedigree B had 18 (FIG. 41), and pedigree J had 9 (FIG. 35).

Age was specified in 93 of the 115 patients. The range of age in these patients was from 2 to 81 years of age, with a mean age of 38.8±20.4. This included 46 females and 47 males.

2. Mortality

During the course of the study, it was known that at least 8 of the 115 patients died. While the exact of date of death and cause were not available for each of these patients, the information available suggested that at least 7 of the 8 patients died of causes unrelated to premature cardiovascular mortality.

Of 4 patients who died in their 9th decade, no cause of death was available for 2 patients, 1 died of pancreatic cancer, and 1 died of sepsis. Four other patients died between the 4th and 7th decade. Of these, 1 died of a brain tumor and 2 died of injuries related to auto accidents. The other patient died at age 62 of coronary artery disease, sepsis, and endocarditis.

3. Visual Acuity

Eighty-four of 93 patients (90%) had a record of BCVA or UCVA. A patient with UCVA of 20/20 was counted as having had both UCVA of 20/20 and BCVA of 20/20 for purposes of calculation of mean visual acuity for the group. Forty-five patients had only BCVA recorded, 30 patients had BCVA and UCVA recorded, and 10 patients had only UCVA recorded (FIG. 43). One patient had UCVA only in 1 eye and BCVA and UCVA in the other eye and so was counted in both categories. Because this patient was counted twice, the total number of patients appeared to add up to 85, even though only 84 patients had a record of BCVA or UCVA.

The mean BCVA and UCVA were analyzed in eyes that did not have prior ocular surgery or documented ocular pathology, such as cataract, amblyopia, macular degeneration, and glaucoma. To calculate the mean BCVA for each of the 3 age-groups, eyes included in the calculation had either a record of BCVA or had UCVA of 20/20 or better.

Of the 149 eyes of 75 patients that had BCVA recorded, ocular pathology excluded 5 eyes in patients <26 years of age, one eye in patients between 26 and 39 years of age, and 38 eyes in patients ≧40 years of age. The mean logMAR BCVA in patients <26 years of age (31 eyes) was 0.084±0.147, at 26 to 30 years of age (39 eyes) was 0.076±0.164, and at ≧40 years of age (35 eyes) was 0.171±0.131.

Of the 78 eyes of 39 patients that had UCVA recorded, ocular pathology excluded 12 eyes in patients ≧40 years of age. The mean logMAR UCVA in patients <26 years of age (32 eyes) was 0.173±0.197; in those 26 to 39 years of age (22 eyes) was 0.125±0.221; and in patients ≧40 years of age (12 eyes) was 0.258±0.144.

The mean Snellen BCVA in affected patients with no other ocular pathology was between 20/20 and 20/25 in those <40 years of age and between 20/25 and 20/30 in those ≧40 years of age. Although there were patients in each age category who achieved BCVA of 20/20 or better, the worst BCVA reported in patients <26 years of age was 20/60, in patients 26 to 39 was 20/70, and in patients ≧40 years of age was 20/100.

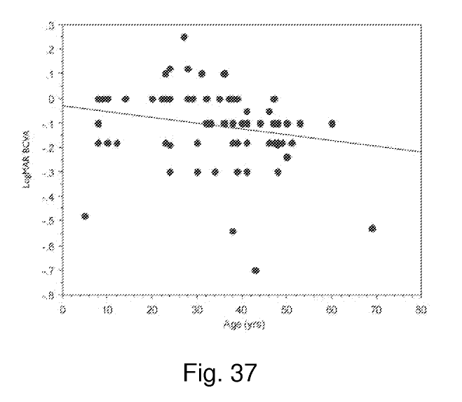

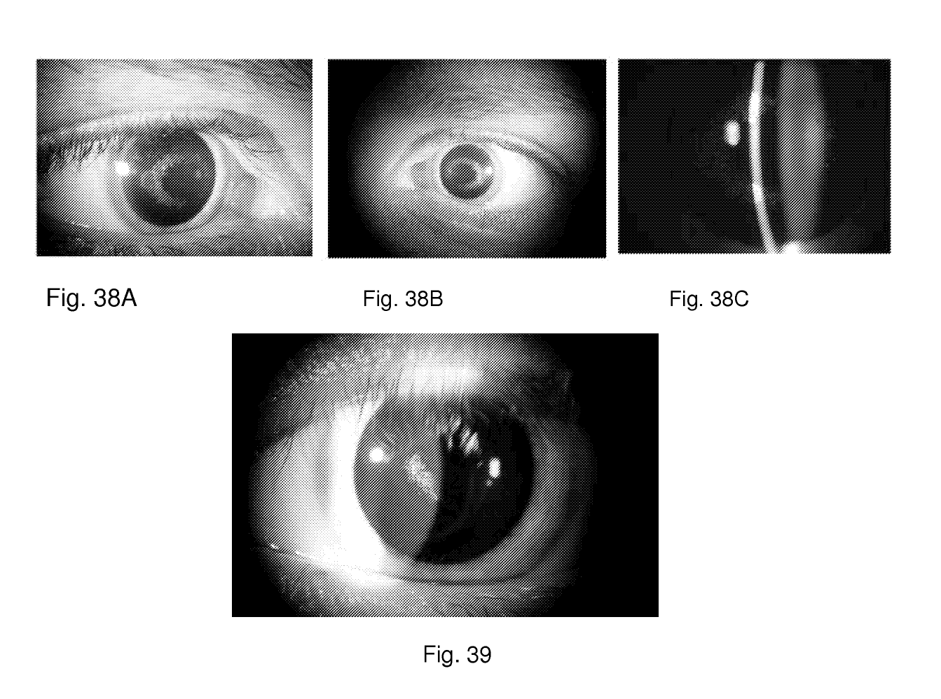

Mean Snellen UCVA was between 20/25 and 20/30 in patients <40 years of age and between 20/30 and 20/40 in patients ≧40 years of age. There were patients in all age categories with UCVA of 20/25, and the worst vision reported in all age categories was UCVA of 20/80. Regression analysis of the vision showed a weak trend of worsening vision with age y=−0.033+0.002×; R²=0.046 (FIG. 37). There was no statistically significant difference between patients <26 years of age and those 26 to 39 for either BCVA (P=0.835) or UCVA (P=0.4101). There was a statistically significant difference for both BCVA (P<0.0001) and UCVA (P<0.0001) between those patients <40 years of age and those ≧40 years of age.

4. Corneal Sensation

Of all eyes enrolled in the study that did not have corneal surgery, only 91 eyes had corneal sensation measurements performed (Table 3), and 47% (43 of 91) had decreased corneal sensation.

Decreased sensation was recorded in 10 of 26 eyes (38%) in patients <26 years of age, in 6 of 22 eyes (27%) of patients between 26 and 39 years of age, and in 27 of 43 eyes (63%) in patients ≧40 years. There was a statistically significant decrease in corneal sensation between those patients ≧40 years of age compared to patients <40 years of age (P=0.004).

The findings in the total cohort were similar to those in the cohort examined by the author. Sixty-seven eyes that did not have prior corneal surgery had corneal sensation measurements that the author personally performed. Twenty-nine of 67 eyes (43%) had decreased corneal sensation measurements. Decreased sensation was recorded in 4 of 12 eyes (33%) of patients <26 years of age, 6 of 20 eyes (30%) of patients 26 to 39, and 19 of 35 eyes (54%) in patients ≧40 years.

These statistics were similar to those found in pedigrees A and B. For patients <26 years of age, decreased corneal sensation was recorded in 2 of 10 patients in Family A and 2 of 8 patients in Family B. Between 26 and 39 years of age, decreased sensation was recorded in 2 of 10 patients in Family A and none of the 6 patients in Family B. In patients ≧40 years of age, decreased corneal sensation was noted in 3 of 7 eyes in Family A and 6 of 12 eyes in Family B.

5. Corneal Findings

a. Crystals

The prevalence of corneal crystal deposition was examined in the total cohort, those patients examined by the author and also in pedigrees A, B, and J. The number of eyes that had documentation of crystalline deposits was compared to the total number of eyes that had a record of presence or absence of crystalline deposits.

In the entire cohort, of the 160 eyes that had no prior corneal surgery and that had notation of presence or absence of corneal crystals, 119 of 160 (74%) had crystalline deposition. The percentage of eyes with crystals varied little among the different age categories with crystals noted in 38 of 50 eyes (76%) of patients <26 years of age, 23 of 36 (64%) of patients 26 to 39 years of age, and 58 of 74 eyes (78%) of patients ≧40 years of age. Four patients had crystalline deposits in only one eye. There was no statistically significant difference in the frequency of crystals reported between the individual age-groups (P=0.25).

If only those patients examined by MDs other than the author were reviewed, 71 of 76 (93%) of eyes had crystal deposits. This compares to crystalline deposits noted in 48 of 84 (57%) of eyes examined by the author with the deposits occurring in 11 of 20 (55%) of eyes in patients <26 years of age, 7 of 20 eyes (35%) of patients 26 to 39 years of age, and 30 of 44 (68%) of eyes of patients ≧40 years of age.

There was a statistically significant higher prevalence of crystals in patients examined by other physicians compared to the prevalence of crystals in patients examined by the author (P<0.0001).

Those pedigrees with 5 or more patients were also examined for crystal prevalence in those patients who had notation of either presence or absence of crystals. Families A, B, and J were examined by the author and had crystalline deposits in 12 of 19 (63%), 11 of 18 (61%), and 3 of 8 (36%), respectively. Both families W and Y, pedigrees from Turkey and Germany, were not examined by the author. Each of these families had 5 affected patients, all of whom had crystalline deposits.

In the younger patients, the crystal configurations were initially often mirror images between the 2 eyes, but the deposits were always subepithelial (FIG. 45). In younger patients, it appeared that the crystals initially formed an arc (FIG. 39) and continued to deposited in ring formation, but by middle age crystals could maintain ring formation (FIG. 40) or be scattered more diffusely (FIG. 41).

b. Central Corneal Haze

Of the eyes examined by all physicians who did not have prior corneal surgery and who had a record of either having presence or absence of central haze, central haze was noted in 11 of 43 eyes (26%) in patients <26 years of age, 28 of 38 eyes (74%) in patients between 26 and 39 years of age, and 71 of 75 eyes (95%) in patients ≧40 years. There was a statistically significant increase in the prevalence of haze between patients <26 years of age and those ≧26 years of age (P<0.0001) and also a statistically significant increase in prevalence of haze between those patients 26 to 39 years of age compared to patients ≧40 years of age (P=0.004)

Of the eyes examined by the author in which a notation was made as to presence or absence of central haze, central haze was present in 6 of 20 eyes (30%) in patients <26 years of age, 18 of 22 eyes (82%) of patients 26 to 39 years, and 47 of 47 eyes (100%) in patients ≧40 years of age. There was an increase in the prevalence of central corneal haze with age, which was statistically significant (P<0.0001).

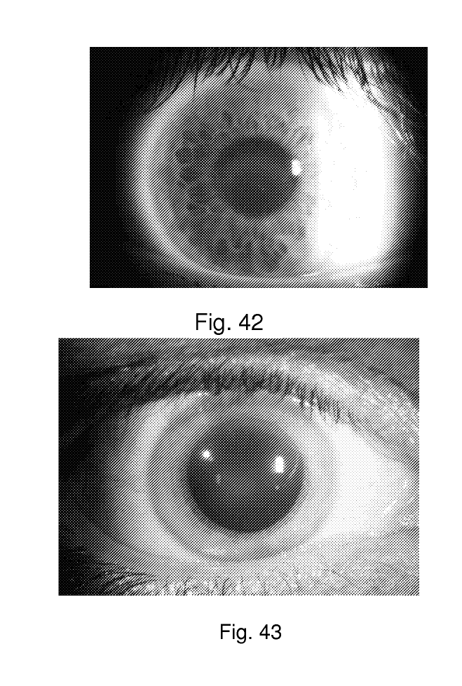

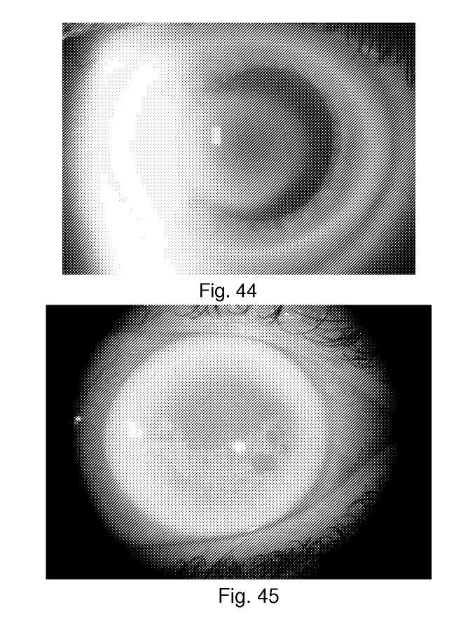

Similar to the ring formation that could occur with crystalline deposition, the central haze could appear in ring formation (FIG. 42), or it could appear as a central disc (FIG. 43). If retroillumination was used, it became apparent that the disc was more lucent centrally (FIG. 44).

c. Crystals/Central Haze

Virtually all patients in each age category had evidence of crystals, central corneal haze, or a combination of both (FIG. 40). In patients without corneal surgery and examined, in all age-groups, 15 eyes had only crystals, 33 eyes had crystals and corneal haze, and 33 eyes had only corneal haze. Three eyes had neither crystal deposition nor corneal haze. The 3 eyes with no central corneal findings belonged to 2 patients, a 4-year-old boy and a 22-year-old man. The 4-year-old child (patient III 4 in FIG. 33) had SCCD crystals in the central cornea of one eye but no manifestations of the disease in his second eye. The 22-year-old man (patient III 1 in FIG. 3) was not diagnosed as having SCCD on his first clinical examination, when his corneas were reported as being clear. Ten years later he was noted to have a subtle central corneal haze in the absence of crystalline deposition, and the diagnosis of SCCD was made.

Of patients without corneal surgery examined by other doctors, in all age-groups, 23 had crystals alone, 32 had crystals and corneal haze, and 11 had only corneal haze. The 3 eyes of the previously described patients had neither crystal deposition nor corneal haze.

Consequently, at all ages, virtually every SCCD patient had either corneal crystals, central corneal haze, or both findings. There was a statistically significant greater number of eyes that had only central corneal haze in patients examined by the author, 33 of 81 eyes (41%), compared to patients examined by other physicians, 11 of 66 (17%) (P=0.0015).

d. Midperipheral Haze

In patients examined in the entire cohort and whose chart notes or photos indicated either the presence or absence of midperipheral haze, none of 44 eyes of patients <26 years of age had midperipheral haze, 9 of 20 eyes (45%) of patients 26 to 39 years of age had midperipheral haze, and 55 of 65 (85%) had midperipheral haze. There was a statistically significant increased prevalence of midperipheral haze in patients ≧40 compared to those <40 (P<0.0001).

Of patients examined by the author, in which chart notes or photos indicated either the presence or absence of midperipheral haze, there was no midperipheral haze in any of the 25 eyes of patients <26 years of age, and midperipheral haze was noted in 2 of 12 eyes (17%) of patients 26 to 39 years of age. The 2 eyes with midperipheral haze belonged to a 39-year-old affected patient. Thirty-five of 39 eyes (90%) of patients ≧40 years of age had midperipheral haze.

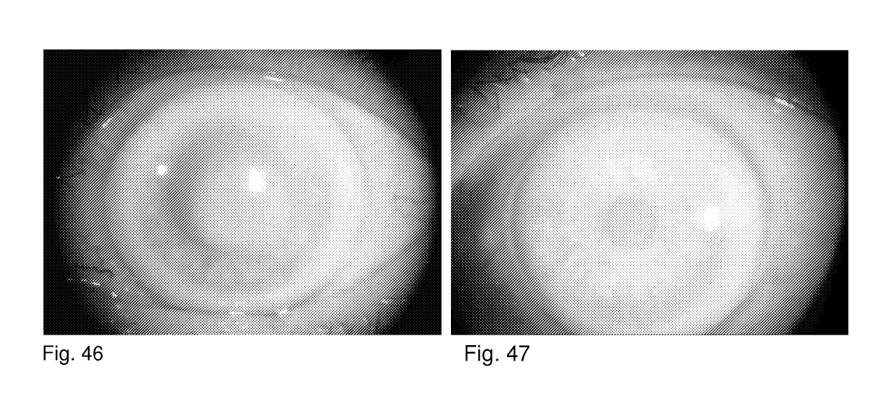

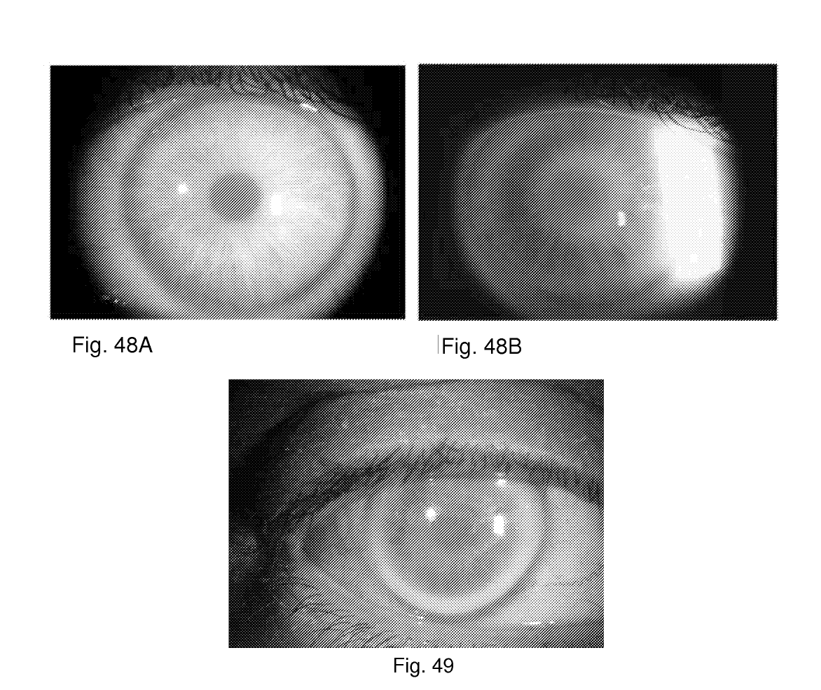

The prevalence of midperipheral haze increased from youngest to oldest age-groups with the majority of patients ≧40 years of age demonstrating this finding. In the older patients sometimes the cornea appeared diffusely hazy with prominent arcus and crystals (FIG. 45) or diffusely hazy with prominent central disc opacity (FIG. 46). There were cases where the most prominent finding was dense diffuse corneal haze (FIG. 47), and it was not possible to delineate central disc opacity. In such cases, the visual acuity could be surprisingly good considering the degree of corneal opacity. In some cases, retroillumination of the diffuse haze revealed that the opacity was not confluent in that there was a denser opacification in the central cornea (FIG. 48).

e. Arcus

Of the all the patients examined whose chart notes or photos indicated either the presence or absence of arcus lipoides, arcus was noted in 10 of 46 eyes (22%) of patients <26 years of age, 36 of 36 eyes (100%) of patients 26 to 39 years of age, and 71 of 73 eyes (97%) of patients ≧40 years of age. There was a statistically significant increased incidence of arcus in patients ≧26 years of age compared to those <26 years of age (P<0.0001).

Of the patients examined by the author whose chart notes or photos indicated the presence or absence of arcus lipoides, in patients <26 years of age, no eyes (0 in 20) had evidence of arcus, while arcus was noted in 20 of 20 eyes (100%) of patients aged 26 to 39 and 47 of 47 (100%) of eyes of patients ≧40 years of age.

The results indicate that virtually all SCCD patients had arcus formation at ≧26 years of age. As the patient aged, the arcus became prominent enough to be easily seen without the aid of a slit lamp (FIG. 49).

6. Long-Term Follow-Up Of SCCD Patients

a. Foreign

Of the 13 foreign families, follow-up examination information was available on only 2 families, X and EE.

b. American

Of the 87 patients affected with SCCD in US pedigrees, at least 8 patients were known to die during the course of the study. An eye and health history questionnaire and medical request form was created to obtain follow-up information on the 79 living American patients.

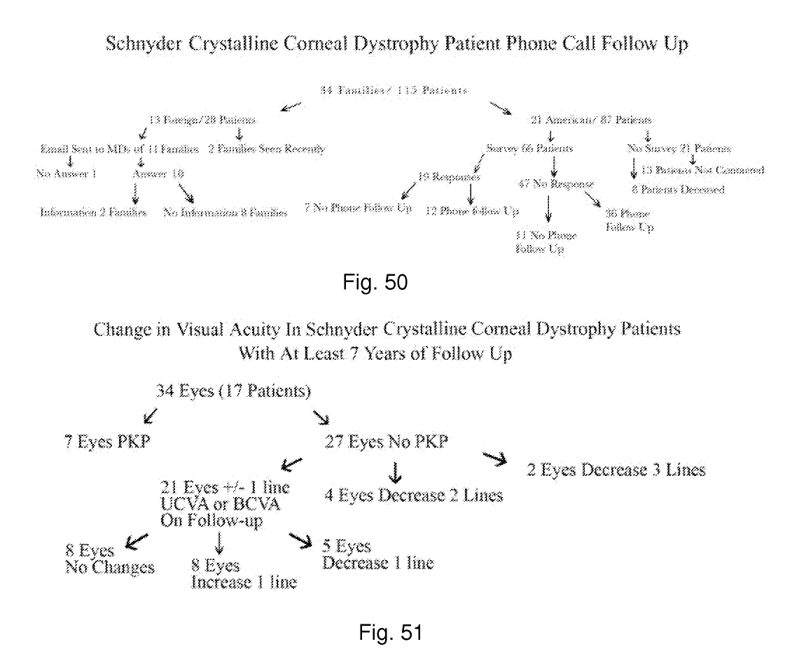

Thirteen of these patients were not sent a request for follow-up data. These included 3 patients from Family Z who were examined for the first time after the survey was mailed, one patient from Family H who had requested to withdraw from the study, and 9 patients from families L, M, S, V, AA, and FF who did not have current addresses or had not answered multiple prior phone or mail requests for information previously (FIG. 50).

The remaining 66 patients were mailed an eye and health history questionnaire as well as a medical record release request. Only 19 patients returned the completed forms and/or the medical record request, which was used to obtain medical records. Twelve of these 19 patients were also contacted by telephone to clarify data.

Of the remaining 47 patients that did not return the written questionnaire or medical record release form, 36 patients answered a phone questionnaire asking about corneal surgery results, systemic cholesterol medication, and information about other family members, including whether any family members had undergone ocular surgery or had died.

In all, 55 of 66 SCCD living patients who were contacted in the United States (83%) answered a phone call or written survey. This represented 55 (70%) of the 79 living American SCCD patients cohort.

Pedigrees A, B, and J, had survey/phone call responses of 15 of 15 living members (100%), 15 of 18 (83%), and 6 of 8 living members, respectively.

7. Visual Acuity Changes With Time in the Individual SCCD Patient

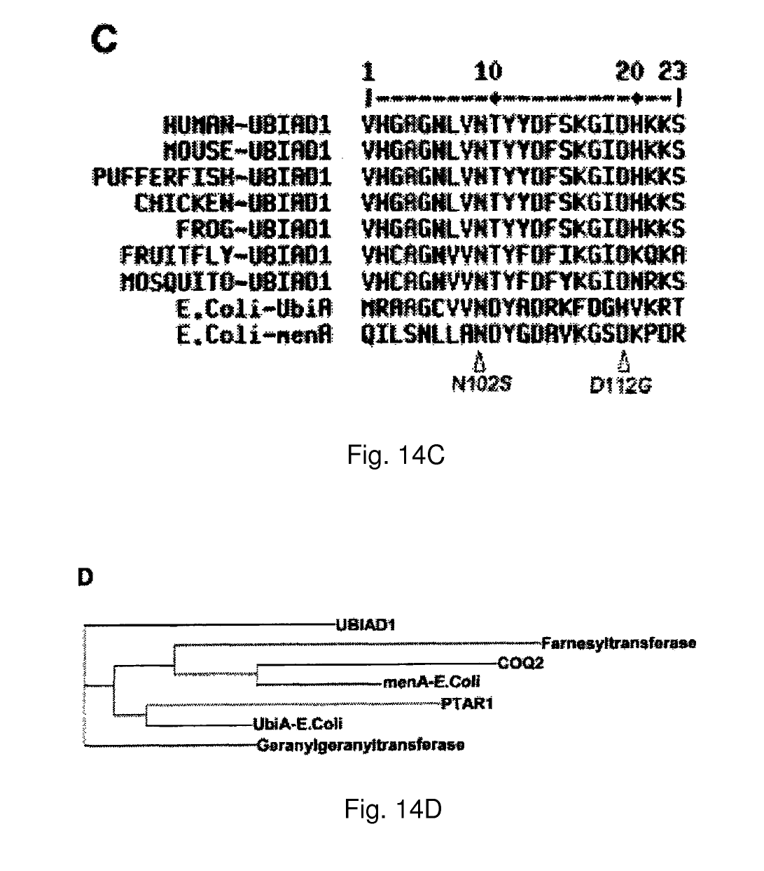

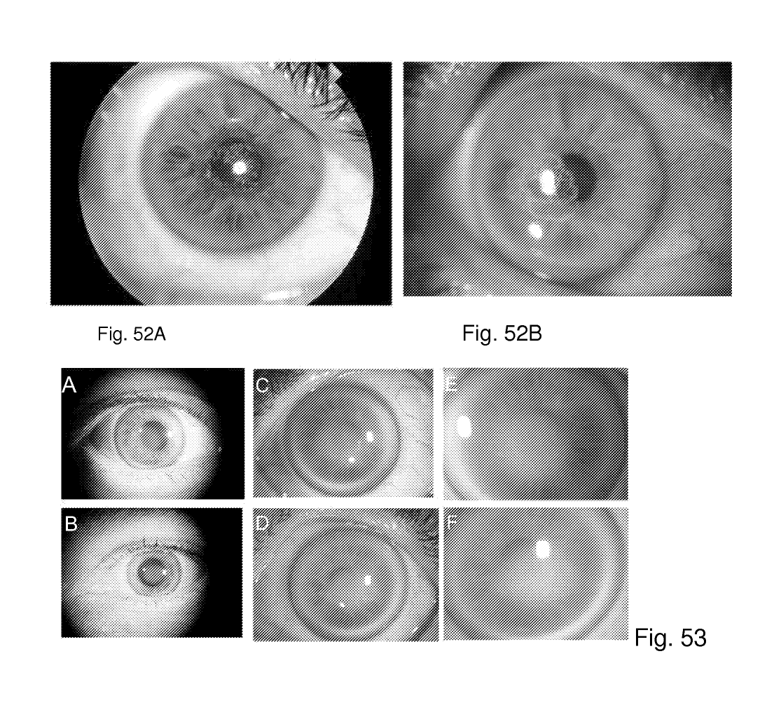

Seventeen patients (34 eyes) had at least 7 years of follow-up from their first to last ocular examination with a mean of 11.4 years±3.9 (range, 7-17) (Table 4). Mean age at initial examination was 33 years±14.7 (range, 8-60) and at last examination was 44.5 years±14.8 (range, 18-67) (FIG. 14). All patients had UCVA or BCVA ≧20/30 on first examination except for a 40-year-old woman in pedigree C with known amblyopia and BCVA of 20/400 and a 38-year-old Taiwanese woman in pedigree X with BCVA of 20/70 OU who subsequently underwent PKP left eye (OS) that year. Four of the 17 patients (24%), 7 of the 34 eyes (21%), with long-term follow-up underwent PKP in the course of the follow-up. A 41-year-old male in Family Q had an unsuccessful PTK that did not improve the BCVA of 20/50, and so a PKP was performed in this eye at age 42 (FIG. 52).

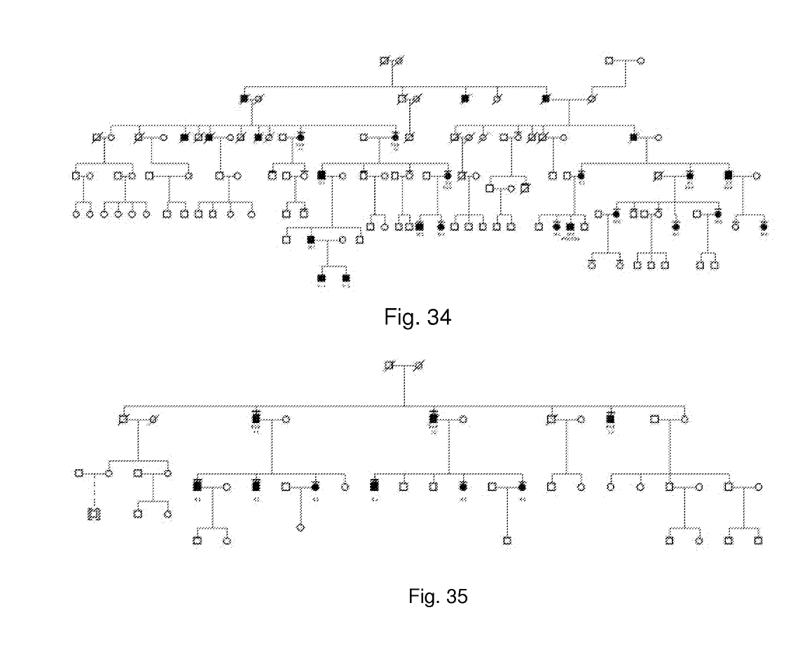

Of 27 eyes that did not undergo surgery, 21 eyes stayed within I line of the initial recorded visual acuity, 8 eyes improved by I line of vision, 8 eyes maintained the same UCVA or BCVA, and 5 eyes lost I line of UCVA or BCVA. Four additional eyes lost 2 lines of BCVA. Three of these eyes had final BCVA of 20/30. In a fourth patient, a 39-year-old woman from Family C; progressive cornea opacification that occurred over a 17-year follow-up caused the BCVA to decrease from 20/30 to 20/50 in her nonamblyopic eye. PKP was reported as being planned in the near future (FIG. 53). Only one patient had a loss of 3 lines of BCVA over 16 years with final BCVA of 20/40 at age 45 (patient III 6 in Family B, FIG. 34).

8. Corneal Surgery

Forty-four corneal surgical procedures were performed on 43 eyes of 29 patients. Twenty-seven patients had PKP and 3 patients had PTK. A 41-year-old male in pedigree Q had PTK on one eye, but when visual acuity did not improve; PKP was performed on the same eye 1 year later (Table 2).

a. PTK

Five eyes of 3 patients had PTK, with bilateral PTK performed in 2 of 3 patients. Mean age was 37 years (range, 34-41). Preoperative BCVA was 20/50 to 20/60 in 4 eyes whose only pathology was SCCD and 20/100 in an eye that also had a preoperative diagnosis of anisometropic amblyopia. BCVA improved in 4 of 5 eyes, including 1 eye that had anisometropic amblyopia.

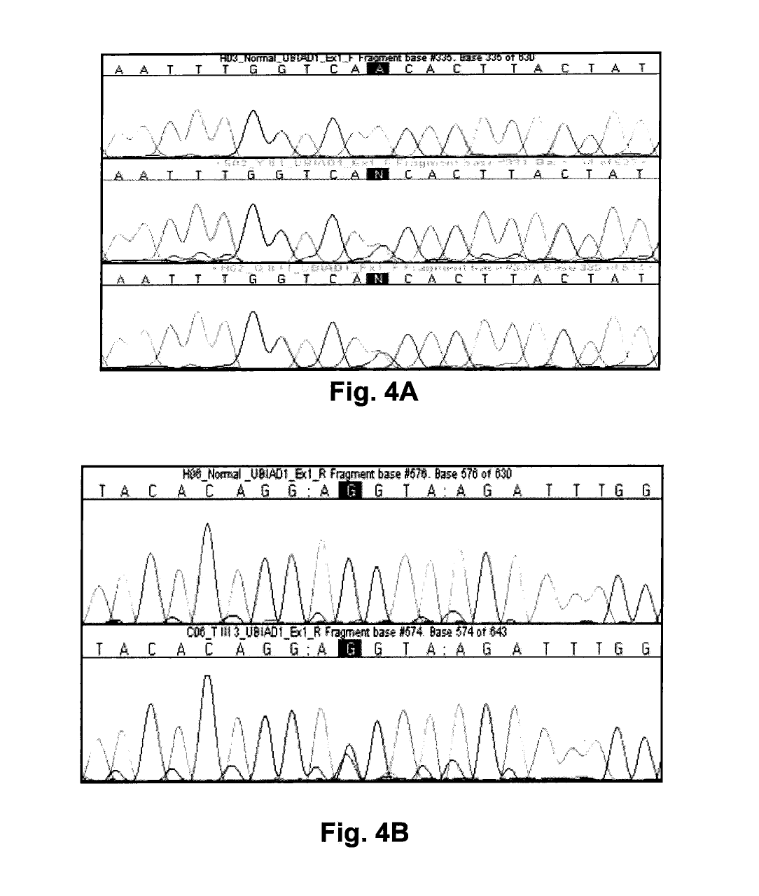

A 34-year-old Turkish man (Family W) had amblyopia OS. H is preoperative BCVA was 20/100 OU, which improved to postoperative BCVA of 20/20 right eye (OD) and 20/50 OS. A 37-year-old man, (patient III 5 in Family B, FIG. 4) underwent PTK and PRK for myopia OU (FIG. 11). The BCVA OD improved from 20/60 to UCVA of 20/25 OD, but postoperative results were not available for the OS. A 41-year-old in Family Q with BCVA of 20/50 had unilateral PTK for corneal crystalline deposition. One year postoperatively, the BCVA was 20/50 with persistence of corneal haze, and PKP was performed (FIG. 52).

b. Age at First PKP

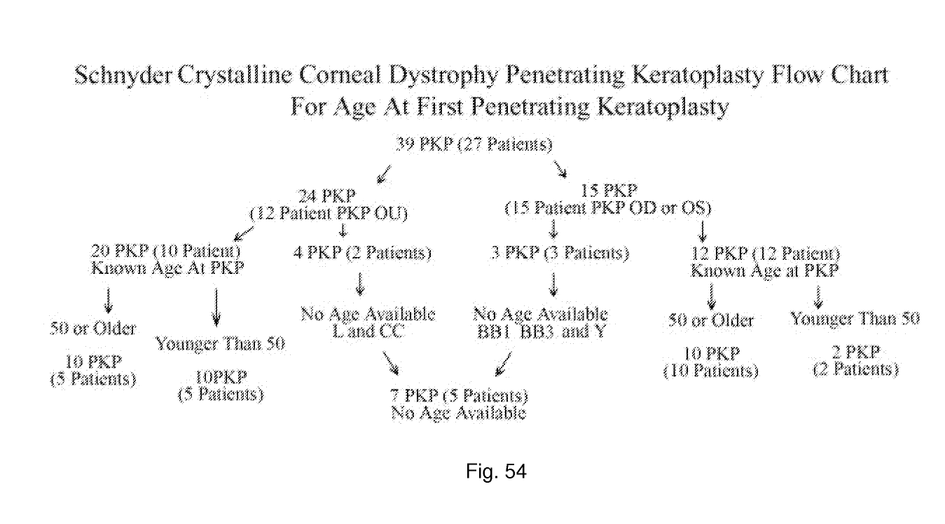

Initial entry examination, subsequent follow-up examinations, e-mail correspondence and written and telephone surveys revealed that 39 PKP were performed in 27 patients. Twelve patients had bilateral PKP. Of the 27 PKP patients there were 12 females and 13 males, and gender was not identified in 2 patients. Age at surgery was known in 22 patients (32 eyes) with a mean age at surgery of 60 years of age±13 years (range, 39-81). Age at surgery was not available in 7 eyes of 5 patients (families L, BB1, BB3, CC and Y) (FIG. 54).

Of the 22 patients whose age was known at first PKP, 15 patients (68%) had their first PKP at ≧50 years of age. The 7 patients <50 at first PKP had a mean age of 43±4 years (range, 39-49). Five of the 7 patients undergoing PKP at a younger age eventually had bilateral surgery compared to the entire cohort, where 5 of 15 had bilateral PKP. There was not a statistically significant difference between the frequency of bilateral PKPs between patients <50 and patients ≧50 years of age (P=0.17).

c. PKP at 50 Years of Age and Above

The most recent eye examination, telephone contact, or questionnaire was used to record the patient's age. For those patients who were deceased, the age at the last examination was recorded as the patient age. Twenty of 37 patients (54%) who were ≧50 years of age on their most recent contact reported having had unilateral or bilateral PKP surgery. For each pedigree, the number of patients ≧50 years of age who had PKP was compared to the total number of patients ≧50 years of age who were members of the pedigree (Table 2). The total number of patients in each pedigree who underwent PKP and PTK were listed in separate columns in Table 2. While information was obtained for each pedigree, only the largest pedigrees A, B, and J had at least 5 patients who were ≧50 years. The prevalence of PKP in the older age-group ranged from 2 of 6 in pedigree A, to 5 of 9 in pedigree B and 3 of 5 in pedigree J. The mean age of those patients in the ≧50 years of age cohort was 62 for A, 67 for B, and 70 for J. Each successive pedigree had both a higher percentage of patients ≧50 who had PKP and a higher mean age for this cohort. However, there was no statistical difference (P=0.79) between the prevalence of PKP in each of the 3 pedigrees.

d. Prevalence of Corneal Surgery With Aging

To determine the prevalence of corneal surgery, PTK or PKP, as the SCCD patient aged, the age of most recent contact (including examination, written survey, or telephone contact) and whether or not the patient reported having had PKP or PTK for SCCD was recorded. In the few cases where the only information available was the age at PKP or PTK, this age was recorded as the actual patient age (FIG. 54).

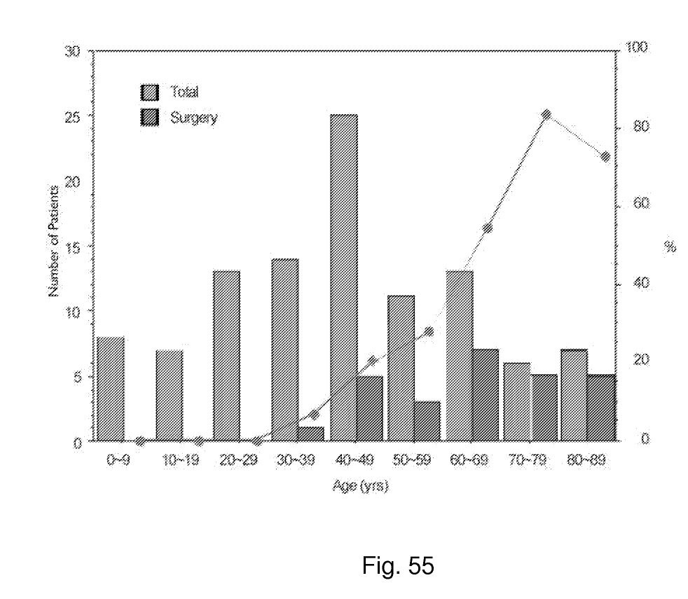

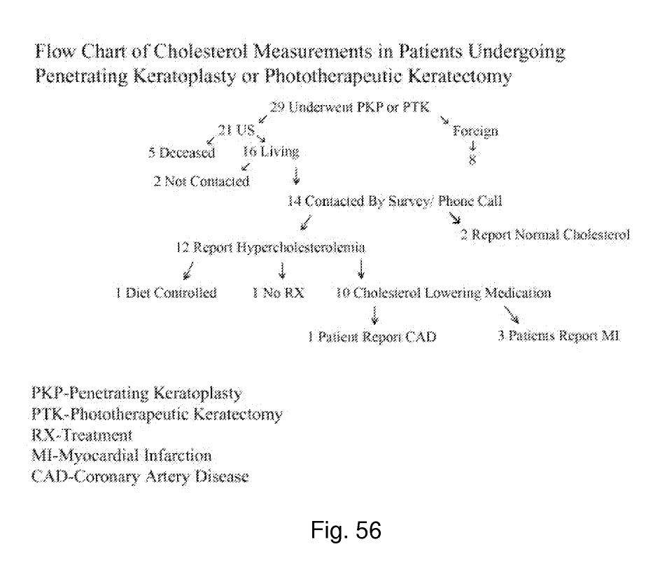

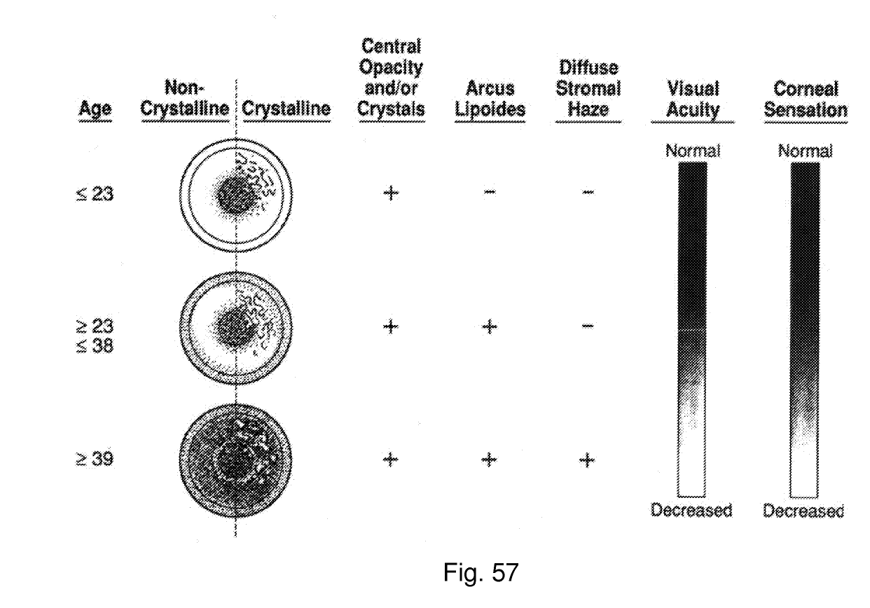

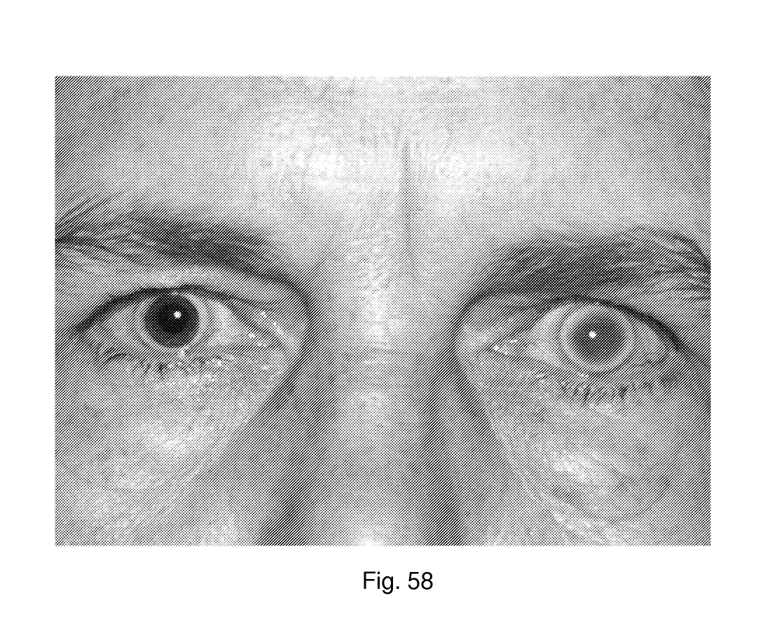

For each decade of age, the number of patients who reported corneal surgery at their last examination was compared to the total number of patients in that age-group. The percentage of patients reporting corneal surgery increased markedly after middle age, with PKP or PTK reported in 1 of 14 patients (7%) in the 4th decade, 5 of 25 (20%) in the 5th decade, 3 of 11 (27%) in the 6th decade, 7 of 13 (53%) in the 7th decade, 5 of 6 in the 8th decade, and 5 of 7 in the 9th decade. There was a statistically significant increase in the prevalence of corneal surgery with age (P=0.002) (FIG. 55).

There were 10 patients in the 8th and 9th decade who had PKP and 3 who had not. The 3 who did not have surgery included a 78-year-old male who lived in Turkey (pedigree W), and no chart notes were available. The 2 additional patients in the 9th decade who did not undergo PKP were siblings in pedigree T. Review of the chart notes indicated that the examining ophthalmologist recorded that PKP was under consideration for both patients because of decreased vision or glare.

e. PKP

Twenty-two patients underwent PKP and had information available about their age at PKP. Preoperative BCVA within 1 year of PKP was available in 9 patients (15 eyes).

### i. Preoperative Vision

Preoperative visual data was unavailable in 13 patients because of the following reasons: Five patients did not sign medical record release forms sent to them although all did communicate medical information by phone or letter, including 3 patients who indicated that they had undergone PKP surgery. Three patients died and old medical records could not be obtained. For the remaining 5, either the patient or physician did not return the follow-up data and there was no other communication. In some cases, while BCVA was available, it was obtained more than 1 year prior to PKP, typically 5 or more years, and so these patients/eyes were excluded from the calculations because they might not give accurate reflection of the level of visual decrease that necessitated surgical intervention.

Preoperative BCVA within 1 year of PKP was available in 15 eyes of 9 patients. Preoperative visual acuity ranged from 20/25 to 20/400 (Table 5), However, 6 of the 15 eyes (4 patients) had evidence of cataract formation and/or macular degeneration, and one eye had prior PTK In the remaining 8 eyes of 5 patients with no other ocular pathology, preoperative BCVA ranged from 20/20 to 20/70 with complaints of glare or decreased contrast recorded for 3 patients from pedigrees, A, E, and G. An additional 2 patients, from pedigrees B and C, had cataract formation with documentation of decrease in vision with glare testing. In total, 5 of the 9 patients (7 PKP eyes) had a chart note indicating either subjective complaint of glare or objective decrease in contrast sensitivity.

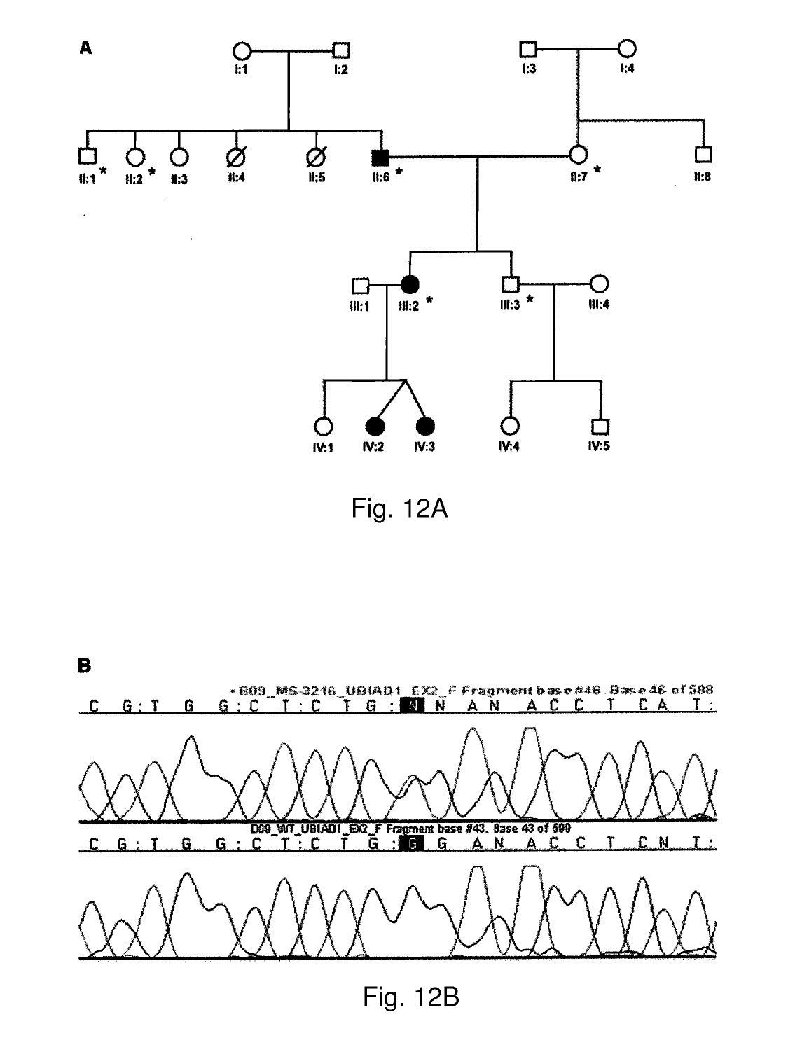

An additional patient who underwent PKP with BCVA 20/30 3 years prior to PKP was not included in the calculations because visual acuity 1 year prior to surgery was not available but was also recorded as having a chief complaint of photophobia preoperatively (FIG. 12).

### ii. Postoperative Vision

Postoperative information was available in 14 patients and 22 eyes. Range of postoperative follow-up was from 1 to 22 years with mean of 6.4±6.7 years. Sixteen of 22 eyes attained BCVA of 20/50 or better. Six eyes attained visual acuity of 20/70 or worse. Five of these eyes had other pathology, including 2 with senile macular degeneration, 1 with Hollenhorst plaque, 1 with graft vascularization, and 1 with a suture abscess at the time of the examination.

Seven patients (11 eyes) recorded had a record of both preoperative BCVA within 1 year of PKP and postoperative BCVA more than 1 year after PKP (Table 6) with a mean follow-up of 5.3 years±2.0 years (range, 1-8). Five eyes had increase of BCVA, 3 eyes maintained same BCVA, and 3 eyes had decrease of I line of BCVA. Of the eyes with visual acuity loss, 2 eyes had evidence of cataract postoperatively and a third had a suture abscess.

Two patients (3 eyes) had BCVA listed as ≧20/30 preoperatively with a presenting complaint of glare or objective decrease in vision on glare testing (Table 5 patients in pedigree A and G). Postoperative BCVA after PKP was the same in 2 eyes and I line worse in the third because of postoperative cataract formation.

f. Recurrence

Five of the 27 patients, 8 of the 39 eyes (21%), who underwent PKP, had evidence of recurrence of the dystrophy in the graft postoperatively. While all of these patients had bilateral PKP, recurrence occurred unilaterally in 2 patients and bilaterally in 3 patients

Visual acuity after recurrence was only available in 2 patients (3 eyes). Two eyes with recurrence had BCVA of 20/40, and the third had BCVA of 20/200 with graft vascularization. The remaining patients with recurrence reported maintenance of good visual acuity despite the recurrence of the dystrophy. There were no cases of repeated PKP performed for dystrophy recurrence.

g. Impact of Hypercholesterolemia in Patients With Corneal Surgery

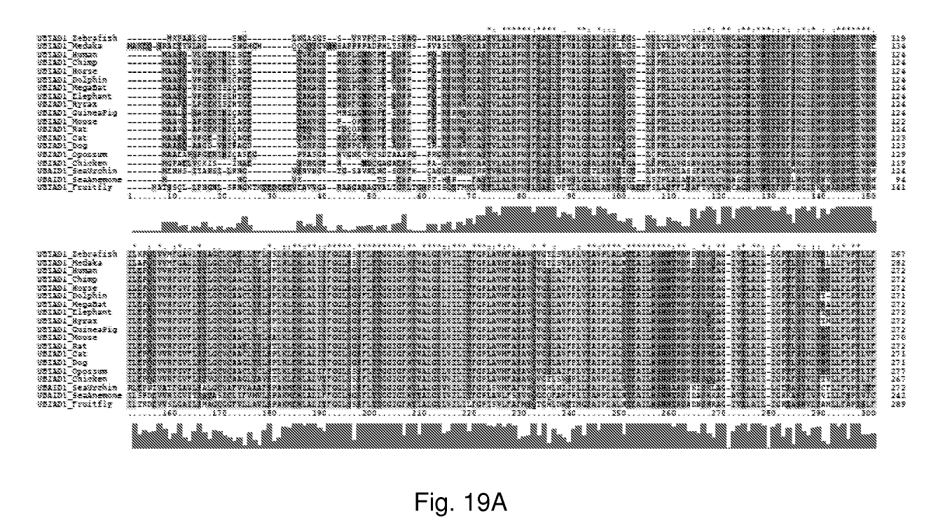

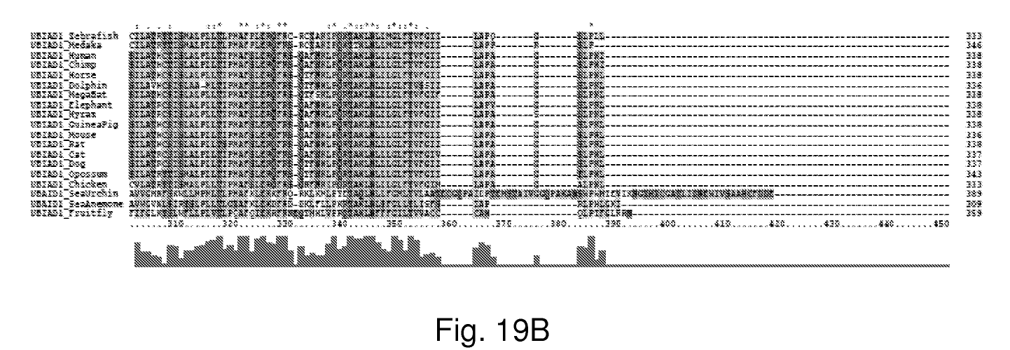

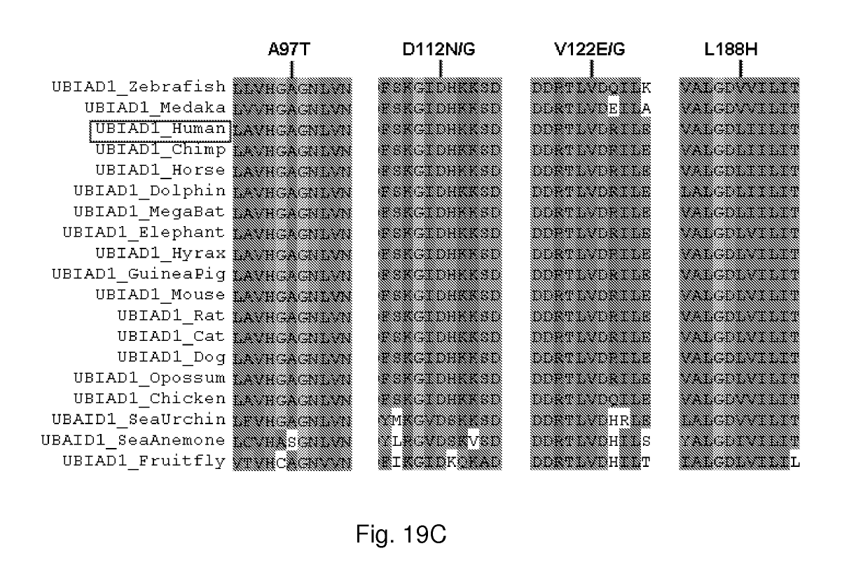

The American cohort who had PTK or PKP was contacted through written and telephone questionnaire to determine the prevalence of hyperlipidemia in those patients who had prior corneal surgery (FIG. 19). Of the 21 American patients who had reported PTK or PKP, 5 patients were deceased. Two additional patients did not receive a mailing or telephone call because of inability to contact them on multiple prior occasions.

Of the 5 deceased patients, 4 were 81 years of age or older at the time of their death. One patient died of pancreatic cancer, 1 patient died of sepsis, and cause of death for the other 2 patients was not available. Two of the four patients in their 9th decade had history of myocardial infarction and congestive heart failure. A fifth patient died at age 62 of coronary artery disease, bacterial endocarditis, and sepsis.

All of the remaining 14 patients were successfully contacted by written or phone questionnaire. Seven patients responded to phone and written questionnaire, and 7 patients responded to phone query alone. Twelve of the 14 patients reported elevated cholesterol (86%). The mean age of the patients with hypercholesterolemia was 68±10.5 years (range, 52-82). Two patients, a 37-year-old and a 52-year-old reported normal cholesterol levels.

Of the 12 patients with hypercholesterolemia, 1 was on diet control, 1 was not using any treatment, and 10 were taking oral cholesterol-lowering medications. Ten of 14 patients (71%) contacted were using an oral medication to lower cholesterol. Cardiovascular disease was reported in 4 of 14 patients (29%) contacted. One patient reported coronary artery disease and three additional patients had a history of prior myocardial infarction

To try to compare prevalence of hypercholesterolemia of patients who had corneal surgery to those who had not undergone PTK or PKP, the frequency of cholesterol-lowering medications in SCCD patients ≧50 years who had not had corneal surgery was compared.

There were 17 patients ≧50 years who had not reported undergoing any corneal surgery. No information on cholesterol values or use of cholesterol medication was available for 4 of these patients, including 1 American patient and 3 foreign patients. Of the 13 patients with information about cholesterol medications, the mean age was 62±10.3 years (range, 50-83). Seven of 13 patients (54%) were taking cholesterol-lowering agents. There was no statistically significant difference between the percentage of patients ≧50 years who were taking cholesterol-lowering agents in the group that had corneal surgery compared to the group that had no surgery (P=0.34).

h. Genu Valgum

While information about genu valgum was not listed for all patients, 5 patients from 3 families (Family A, Z, and M) were documented to have genu valgum. This finding occurred in at least 5 of 115 patients enrolled, or approximately 4% of patients.

### E. Data Analysis

1. The Basics

Different cohorts were analyzed to confirm or refute trends to minimize the possibility of bias.

For trends involving changes of visual acuity, corneal findings, or surgical intervention with age, there were 4 types of cohorts used. The entire cohort of patients with ages specified (93 patients) was always analyzed because this provided the largest cohort and increased statistical power. Data was compared to the cohort of patients examined by the author personally (47 patients) because this cohort provided consistency of examination technique as all patients were examined by the same doctor. The largest pedigrees, A, B, and J were also examined because the follow-up of all available members of an individual family might decrease selection bias. Finally, analysis of the cohort of patients examined by physicians other than the author (46 patients) provided a means to detect a difference in examination technique by the author versus other physicians or, alternatively, detect a difference in type of patients seen by the author versus other physicians.

When there were similarities between the findings among the groups, conclusions appeared to be confirmed, but when there was a difference among the groups, the data was further analyzed. For example, comparison of the cohorts revealed that 57% of patients examined by the author had crystals compared to crystalline deposits noted in 93% of patients examined by other physicians.

To clarify this large difference in findings, the largest pedigrees were examined. Pedigrees A, B, and J had crystalline deposition in 12 of 19 (63%), 11 of 18 (61%), and 3 of 8 patients, respectively, but most patients were examined by the author. The only pedigree that had 5 or more members with data about crystals that was not examined by the author was pedigree W from Turkey and Y from Germany. In both families, all family members (100%) had crystalline deposits. The possible explanations for this variation in findings were either that the families the author examined had different clinical manifestations than those examined by others physicians or that the author has a higher index of suspicion to make the diagnosis of SCCD in patients who lacked the characteristic crystalline deposition.

The second challenge was determination of the incidence of PKP in SCCD. A critical question to address initially was whether the selection of the study population had introduced unacceptable bias. Perhaps patients with the most severe disease were referred for entry into the study.

If this was the case, the number of patients undergoing PKP would be inordinately high. The unwanted result of this preselection could be an inaccurately dismal prediction of the natural history of the disease by suggesting a higher surgical intervention than actually occurs. However, it was also possible that an insufficient follow-up of the cohort could result in the underreporting of PKPs. This could result in a falsely optimistic picture of the disease course.

An attempt to answer this challenge was the separate analysis of the 3 largest pedigrees, which had not only the greatest number of patients examined in each family but also the highest response to the phone and written follow-up questionnaires.

Pedigrees A, B, and J had long-term follow-up ranging from 75% to 100%. Consequently, the prevalence of PKP in these large pedigrees with better long-term follow-up was compared to the entire cohort to see if the results were consistent. In the entire cohort, 20 of 37 patients (54%) aged ≧50 years reported prior PKP. The prevalence of PKP in patients aged ≧50 ranged from 2 of 6 in pedigree A, 5 of 9 in pedigree B and 3 of 5 in pedigree J with the pedigrees with higher PKP incidence having a higher mean age. There was no statistically significant difference between the frequency of PKP in these 3 pedigrees (P=0.79)

Despite the many limitations of this study, there appeared to be a consistency of trends of corneal surgical intervention, BCVA, and corneal findings with age, which suggest the accuracy of the conclusions drawn.

2. Genetics

SCCD is inherited as autosomal dominant trait with high penetrance and has been mapped to the UBIAD1 gene on 1p36. (Shearman, et al., Hum Mol Genet. 1996; 5:1667-1672; Aldave, et al., Mol Vis 2005; 11:713-716; Theendakara, et al., Hum Genet. 2004; 144:594-600; Riebeling, et al., Opthalmologe 2003; 100:979-983; Weiss, et al., Invest Opthalmol Vis Sci 2007; 48:5007-5012).

Although most cases of SCCD have a clear pattern of heredity, sporadic cases have been reported. (Brownstein, et al., Can J Opthalmol 1991; 26:273-279; Weller, et al., Br J Opthalmol 1980; 64:46-52; Delleman, et al., Opthalmologica 1968; 155:409-426; Kohnen, et al., Klin Monatsbl Augenheilkd 1997; 211:135-136; Bonnet, et al., Bull Soc Ophtalmol Fr 1934; 46:225-229; Burns, et al., Trans Am Opthalmol Soc1978; 76:184-196; Gillespie, et al., Am J Opthalmol 1963; 56:465-467). Three of the 34 families, families E, G, and H, reported no history of the disease in prior generations. Although this could not be confirmed because both parents of the proband were not available for examination, the disease appeared to be sporadic by history in these 3 families.

3. Ethnicity

While the ethnicity of the patients in the literature with SCCD is largely Caucasian, Asian patients with SCCD have also been reported. (Kajinami, et al., Nippon Naika Gakkai Zasshi 1988; 77:1017-1020; Yamada, et al., Br J Opthalmol 1998; 82:444-447; Wu, et al., Opthalmology 2005; 112:650-653). In this study, patients were Caucasian, Asian, and African American. For convenience, Family W from Turkey was classified as Caucasian. There are no published articles reporting the occurrence of SCCD in the African American population. Although the initial pedigrees examined, A, B, C, and D were Swede-Finn, the majority of the other US pedigrees did not have Swede-Finn ethnicity. Pedigrees E and J reported Hungarian ancestry, and pedigree Z was from Kosovo. The other pedigrees did not provide information about their ancestry.

### F. Diagnosing SCCD

1. Corneal Biopsy

The corneal findings in SCCD are well described in the literature. Nevertheless, determining whether an individual patient has the disease can be difficult, not only because of the rarity of the disease, but also because confusion is introduced by misinformation published about diagnostic criteria. Despite the predictable clinical findings in this dystrophy, as recently as the last decade, 2 articles were published using corneal biopsy rather than slit-lamp examination in order to establish the diagnosis. (Brownstein, et al., Can J Opthalmol 1991; 26:273-279; Ingraham, et al., Opthalmology 1993; 100:1824-1827). Ciancaglini wrote that “the diagnosis of SCCD is usually based on clinical findings and corneal biopsy.” (Ciancaglini, et al., J Cataract Refract Surg 2001; 27:1892-1895). This present data on SCCD will not only clarify the long-term history of this disease but serve to further clarify the clinical findings of this disease so that corneal biopsy will not be required.

SCCD causes progressive corneal opacification with age. Grop described 17 patients ranging in age from 7 to 82 and observed that patients developed an arcus by age 20, a central opacity at age 30, and a diffuse opacity at age 40. (Grop, Acta Opthalmol Suppl (Copenh) 1973; 12:52-57). Despite the increasing corneal opacification, he reported that good vision was maintained until the 50s or 60s.

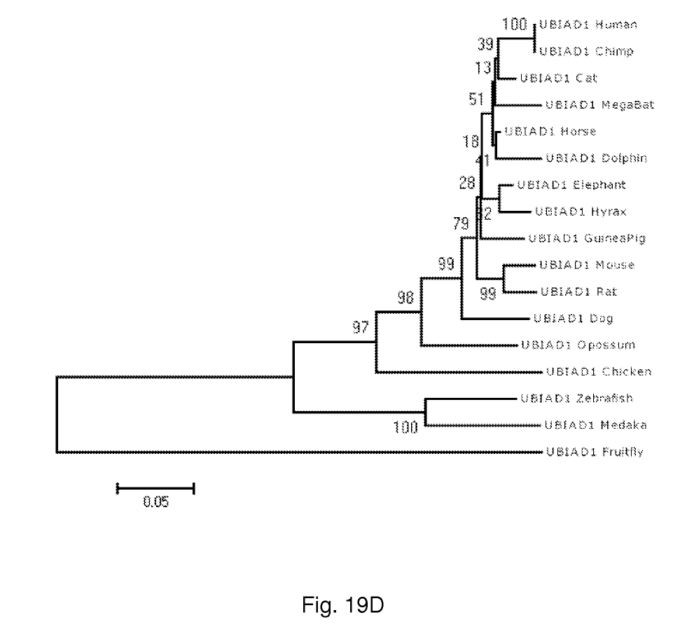

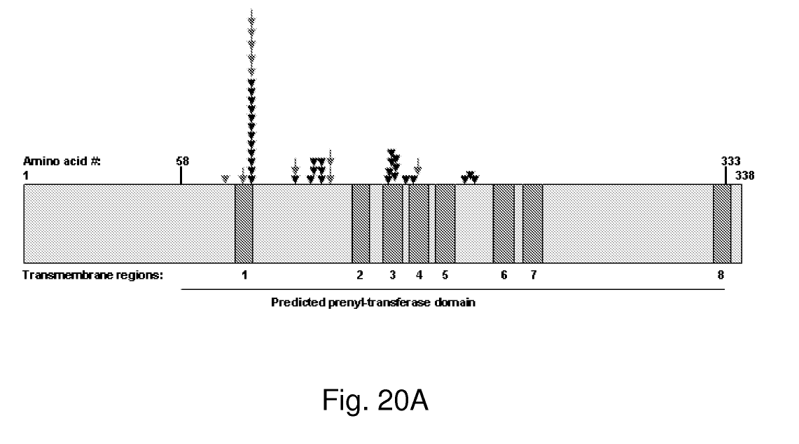

A slightly different schema was published based on the initial examination of 18 affected patients with SCCD in the 4 large Swede-Finn pedigrees (Weiss, Cornea 1992; 11:93-101). In this article, the central opacity was described to occur first in patients less than 23 years of age, the arcus was present in affected patients between 23 and 37, and those patients older than 37 developed a midperipheral corneal opacification (FIG. 20). The present report corroborates most of these prior findings on the course of progression of the corneal findings in the disease. The earliest finding was either a central corneal opacity and/or crystalline deposition. Virtually all patients had one or both of these findings in all age-groups.

Often the central opacity would have a ringlike formation that allowed the central visual axis to be spared until later in life. Crystals initially appeared to deposit as a ring. The central corneal haze could also be deposited as a ring or as a disc. In early SCCD with central corneal disc like opacification; retroillumination often revealed that the opacity was less dense centrally. Even later in life, the central opacity appeared to be the least dense at its center, when viewed with retroillumination. Delleman and Winkelman described different patterns of corneal opacification in SCCD, including a ringlike central deposit.

While arcus lipoides was recorded in 10 of 26 eyes (22%) of the patients <26 years of age in the entire cohort and none of the patients <26 years of age examined by the author; 71 of 93 eyes (97%) of patients in the entire cohort and 47 of 47 eyes (100%) examined by the author in patients who were ≧26 years of age had arcus lipoides.

Quantification of midperipheral haze was more challenging because information about this finding was often not recorded, but examination revealed that no patients <26 years of age had midperipheral haze, 9 of 20 eyes (45%) had arcus between ages 26 and 39. By ≧40, 55 of 65 eyes (85%) had midperipheral haze. This finding was more difficult to determine in the individual patient because it represented the overall progression of corneal opacification that occurs with time in the SCCD cornea. However, there was a statistically significant increase of midperipheral haze in patients ≧40 compared to those <40 (P<0.0001).

This clarification of the corneal changes that developed with age underscores that the major clinical finding in SCCD was a diffuse progressive corneal opacification. Progressive diffuse corneal opacification in SCCD has been previously reported. As the corneal opacity became more dense, even patients in SCCD pedigrees could observe the corneal opacification with their naked eye. The progressive corneal changes allowed patients to report which family members had “cloudy” corneas.

2. Crystals and Diagnosing SCCD

For decades, the literature has reflected that an integral part of SCCD diagnosis was the deposition of cholesterol crystals. The importance of crystals in making the diagnosis of SCCD was first challenged in 1993, when examination of 4 large SCCD pedigrees revealed only 50% of patients had cholesterol crystal deposition. Nevertheless, the majority of published articles about SCCD describe the corneal crystalline change (Lisch, et al., Ophthalmic Paediatr Genet. 1986; 7:45-56; Sysi, Br J Opthalmol 1950; 34:369-374; Bron, et al., Br J Opthalmol 1972; 56:383-399; van Went, et al., Niederl Tijdschr Geneesks 1924; 68:2996-2997; Schnyder, Schweiz Med Wochenschr 1929; 10:559-571; Schnyder, Klin Monatsbl Augenheilkd 1939; 103:494-502; Bec, et al., Bull Soc Ophtalmol Fr 1979; 79:1005-1007; Chem, et al., Am J Opthalmol 1995; 120:802-803; Delogu, Ann Ottalmol Clin Ocul 1967; 93:1219-1225; DiFerdinando, G Ital Oftalmol 1954; 7:476-484; Freddo, et al., Cornea 1989; 8:170-177; Garner, et al., Br J Opthalmol 1972; 56:400-408; Grop, Acta Opthalmol Suppl (Copenh) 1973; 12:52-57; Hoang-Xuan, et al., J Fr Ophtalmol 1985; 8:735-742; Kaden, et al., Albrecht Von Graefes Arch Klin Exp Opthalmol 1976; 198:129-138; Lisch, Klin Monatsbl Augenheilkd 1977; 171:684-704; Mielke, et al., Opthalmologe 2003; 100:158-159; Rodrigues, et al., Am J Opthalmol 1987; 104:157-163; Thiel, et al., Klin Monatsbl Augenheilkd 1977; 171:678-684; Weller, et al., Br J Opthalmol 1980; 64:46-52) although diagnosis of the disease in absence of crystals is also described. (Lisch, et al., Ophthalmic Paediatr Genet. 1986; 7:45-56; Bron, et al., Br J Opthalmol 1972; 56:383-399; Bec, et al., Bull Soc Ophtalmol Fr 1979; 79:1005-1007; Grop, Acta Opthalmol Suppl (Copenh) 1973; 12:52-57; Delleman, et al., Opthalmologica 1968; 155:409-426; Weiss, et al., Opthalmology 1992; 99:1072-1081).

McCarthy and coworkers described a 62-year-old with bilateral corneal clouding with history of poor vision in both of the deceased parents and corneal opacification in the patient's daughter. (McCarthy, et al., Opthalmology 1994; 101:895-901). There were no crystals present, and despite the apparent autosomal dominant inheritance, the patient received a diagnosis of macular dystrophy, which is an autosomal recessive inherited corneal dystrophy. Histopathology demonstrated lipid infiltration characteristic of SCCD but absence of alcian blue staining Alcian blue stains mucopolysaccharides, which are deposited in macular dystrophy. Consequently, the histopathological staining pattern was characteristic of SCCD, not macular dystrophy, despite the initial misdiagnosis on clinical examination.

Previously, many thought that the presence of crystals was integral to the diagnosis of SCCD. In 1972, Garnernd Tripathi (Garner, et al., Br J Opthalmol 1972; 56:400-408) wrote about a SCCD case described by Offret (Offret, et al., Arch Ophtalmol Rev Gen Ophtalmol 1966; 26:171-181) that “must be accepted with some reservation since cholesterol crystals were not demonstrated.” Unfortunately, the incorrect presumption that a patient cannot have SCCD unless crystals are present is still fairly prevalent. Even more recent literature indicates that the disease is characterized by presence of crystals and that while a noncrystalline form occurs, it is much less common (Paparo, et al., Cornea 2000; 19:343-347) or that “the main features . . . crystalline spindle shaped deposits.” (Ciancaglini, et al., J Cataract Refract Surg 2001; 27:1892-1895).

Perhaps, then, it should not have been surprising to discover the large difference in the prevalence of crystalline deposition between patients examined by the author, who found crystals in 57% of the eyes examined, compared to the other physicians, who reported crystals in 93% of eyes they examined. While one possible explanation was that the Swede-Finn pedigrees of A, B, C, and D examined by the author could have had different manifestations of the dystrophy than the majority of the pedigrees; pedigree J with Hungarian ancestry was also examined by the author. The majority of members of this pedigree also did not have crystals. Typically, photographs of the patients who were not examined by the author appeared to have similar changes as those patients examined by other physicians. For example, the slit-lamp photo of the corneal changes in 38-year-old Taiwanese female were similar to the changes in a 38-year-old American male (FIG. 40).

Another possible reason why the author saw more patients with SCCD without crystals is that cases of SCCD without crystals were not diagnosed by others. The challenge of making the diagnosis of SCCD in these patients has been previously reported. The detection of early central panstromal haze in a patient with early SCCD without crystals is very difficult. The author initially misdiagnosed a 23-year-old male in pedigree A (Patient III 1 in FIG. 33) as being unaffected because no corneal opacification or crystals were detected on slit-lamp examination. Genetic testing subsequently revealed that the patient had the defect on chromosome 1 indicating he was affected with SCCD. Repeated slit-lamp examination when the patient was age 30 revealed extremely subtle signs of central corneal clouding and arcus bilaterally. Even at that age, it would have been easy to dismiss the subtle corneal clouding that was noted on examination if the examiner had not prior knowledge about the history.

It is not possible to determine whether the pedigrees examined by the author had different disease manifestations or whether the acrystalline form of the disease was not diagnosed by referring physicians. However, other findings, such as average BCVA, loss of BCVA over time, and age at surgical intervention, did not seem to vary between the pedigrees.

The increased incidence of PKP with age was associated with the progressive corneal opacification that is characteristic of the disease. It is important to emphasize that despite the emphasis on corneal crystalline deposition in SCCD, which may or may not be present in an individual patient, all patients manifest the finding of progressive corneal clouding. Some patients with SCCD who lack the characteristic corneal crystals consult with many ophthalmologists, including corneal specialists, in their quest for a diagnosis. The difficulties experienced by multiple members of Family J who did not obtain a definitive diagnosis for the corneal clouding even after undergoing PKP, illustrate the problem.

3. Two Families With Clinical and Histopathologic Misdiagnosis

A 74-year-old male from Family J (patient I 1 in FIG. 35) had an inability to find out why family members had “cloudy cornea” despite examinations over the past 10 years by multiple well-respected corneal specialists. Both he and 2 brothers had even undergone successful PKPs, but no conclusive diagnosis was obtained from the histopathologic examination of corneal specimens. The patient was taking a cholesterol-lowering agent for hypercholesterolemia and reported a strong family history of “cloudy eyes.” Despite diffuse cornea clouding OD, which made it difficult to examine anterior segment structures (FIG. 47), the BCVA was surprisingly good at 20/25 OD. He had a clear corneal transplant OS, but BCVA was reduced to 20/40 in this eye because of a Hollenhorst plaque. Although the corneal haze was diffuse without a clearly defined central opacity and an arcus which appeared to blend into the diffuse cornea haze, the corneal findings were consistent for SCCD without crystal deposition

Other members of the patient's family (pedigree J) were examined. The patient's 80-year-old brother (patient I 2 in FIG. 35) had a PKP OD 3 years previously for corneal clouding, and chart notes revealed the corneal specialist listed the diagnosis in this eye as central cloudy dystrophy of Francois (CCDF). On postoperative examination, BCVA was 20/30 OD and 20/40 OS. The PKP OD was clear, whereas the corneal examination OS showed diffuse corneal clouding slightly more prominent centrally and no crystalline deposits (FIG. 59). The stromal opacification was tessellated, which was similar to that seen in CCDF or posterior crocodile shagreen. Tessellation of the corneal opacity in SCCD has been previously reported. (Wu, et al., Opthalmology. 2005; 112:650-653). Review of slit-lamp photos of the patients with SCCD examined in this study revealed members of pedigrees A, B, C, G, J, and X with a central opacity that contained polygonal opacities similar to posterior crocodile shagreen or CCDF. It was not possible to determine whether the polygonal opacities represented an additional corneal degeneration, posterior crocodile shagreen, or just another pattern of morphology of lipid deposit. In addition, the fact that the 80-year-old patient was having visual disability associated with the corneal clouding argued against CCDF, because CCDF is reported to cause no visual disability. (Bramsen T, et al., Acta Opthalmol (Copenh) 1976; 54:221-226; Karp, et al., Arch Opthalmol 1997; 115:1058-1062; Meyer, et al., Cornea 1996; 15:347-354; Strachan, et al., Br J Opthalmol 1969; 54:192-194).

The histopathology report from the 74-year-old's prior PKP surgery was requested. The preoperative pathology diagnosis was corneal opacity. Postoperative pathology diagnosis was endothelial corneal degeneration with bullous keratopathy and central corneal leukoma. The slide was reviewed, and it appeared that the endothelium could have been stripped in processing, which gave the misdiagnosis of bullous keratopathy; no central scarring was noted. It was difficult to make any specific diagnosis on basis of re-review of the specimen because the prior routine processing of the slide prevented subsequent stains for lipid.

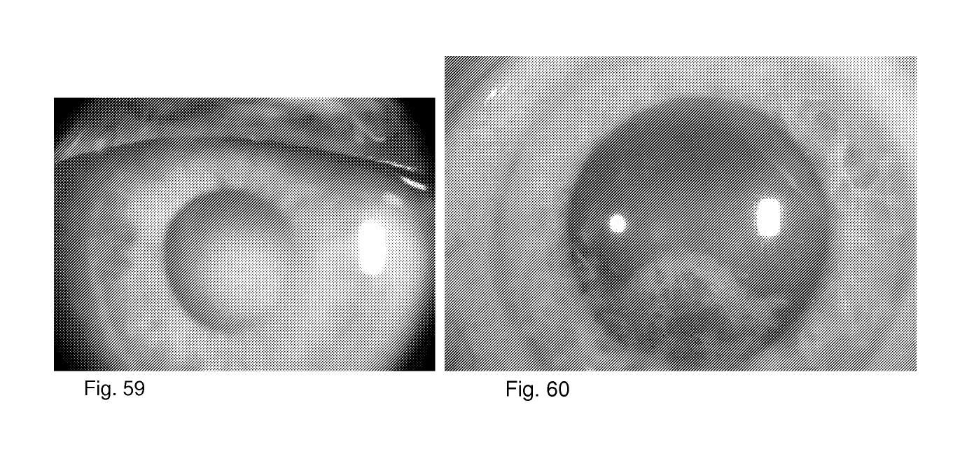

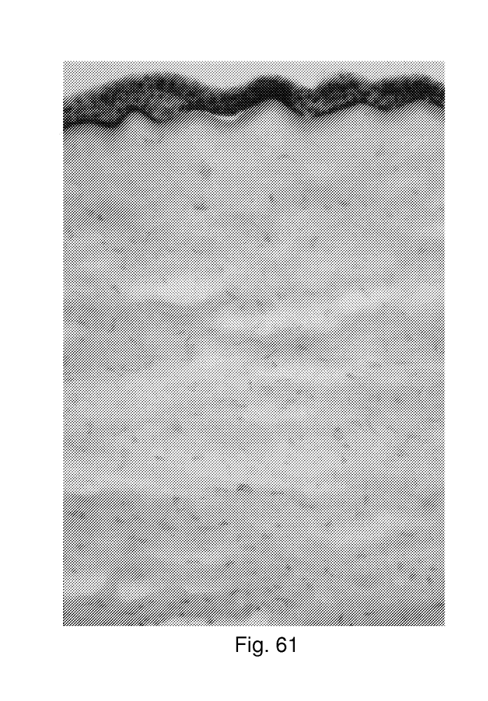

The son (patient II 1 in FIG. 35) of the initial patient was examined with BCVA of 20/25 OD and 20/50 OS. There was a history of amblyopia OS and evidence of cataract formation OU. Corneal examination revealed bilateral central corneal opacity, subepithelial corneal crystals, midperipheral haze, and arcus (FIG. 60).

In total, the author found that 9 members of the pedigree had SCCD with bilateral corneal opacification with 3 of 9 patients having cholesterol crystalline deposition on initial examination.

Should the diagnosis of SCCD have been apparent initially? The 80-year-old proband reported that he had seen 5 corneal specialists throughout the prior decades and was unable to obtain a definitive diagnosis. While the constellation of clinical findings in the 2 brothers was challenging, namely the absence of crystal deposition and the diffuseness of the corneal changes, they were within the spectrum of SCCD findings. The patients had a history suggestive of autosomal dominant inheritance, hypercholesterolemia, corneal opacification so severe that the patient himself could remember other family members with corneal clouding, and BCVA that appeared disproportionately good compared to the severity of the opacity. All of these findings were highly suggestive, if not diagnostic, of SCCD.

4. Why Histopathology in SCCD Does Not Always Yield the Diagnosis

Unfortunately, the histopathologic changes associated with abnormal lipid deposition in the cornea can be missed if the specimen is not processed properly. If the ophthalmologist does not suspect the disease and alert the pathologist, the opportunity to make the diagnosis can be lost because the lipid can be dissolved by routine processing.

The inability to obtain accurate pathology was also observed to occur in a patient from pedigree U, who reported that he could see the “arch around” his father's eye “but no clouding.” Correspondence with the patient indicated that at age 30, he was initially diagnosed at “a reputable university eye clinic” to have “atypical granular dystrophy.” He wrote that “years later, it was changed to Schnyder” during an examination with “two well respected corneal specialists.” PKP was performed, but no indication of the suspected clinical diagnosis was written on the pathology specimen. The final pathology report indicated “focal loss of endothelial cells consistent with Fuchs endothelial dystrophy.” No lipid stains were performed.

Ophthalmologists are cautioned of the importance of alerting the pathologist when considering a diagnosis of sebaceous cell carcinoma because without the proper preparation of the specimen, lipid can dissolve and the opportunity to make the diagnosis with lipid stains can be lost. If tissue is not embedded properly, staining for lipids can be negative because the lipids are dissolved out during the dehydrating stage of embedding (Hoang-Xuan, et al., J Fr Ophtalmol 1985; 8:735-742).

Without proper preparation of the corneal specimen in SCCD to avoid fixatives that dissolve the lipid, the opportunity to do special staining in SCCD can be lost as well.

### G. Histopathology

1. Light and Electron Microscopy

Histopathology of SCCD has been well described with abnormal lipid deposition throughout the corneal stroma. (Brownstein, et al., Can J Opthalmol 1991; 26:273-279; Freddo, et al., Cornea 1989; 8:170-177; Garner, et al., Br J Opthalmol 1972; 56:400-408; Weller, et al., Br J Opthalmol 1980; 64:46-52; Delleman, et al., Opthalmologica 1968; 155:409-426; Weiss, et al., Opthalmology. 1992; 99:1072-1081; Bonnet, et al., Bull Soc Ophtalmol Fr 1934; 46:225-229; Offret, et al., Arch Ophtalmol Rev Gen Ophtalmol 1966; 26:171-181; Babel, et al., Arch Ophtalmol Rev Gen Ophtalmol 1973; 33:721-734; Ehlers, et al., Acta Opthalmol (Copenh) 1973; 51:316-324; Ghosh, et al., Can J Opthalmol 1977; 12:321-329; Malbran, Am J Opthalmol 1972; 74:771-809; Pfannkuch, Klin Monatsbl Augenheilkd 1978; 173:355-358).

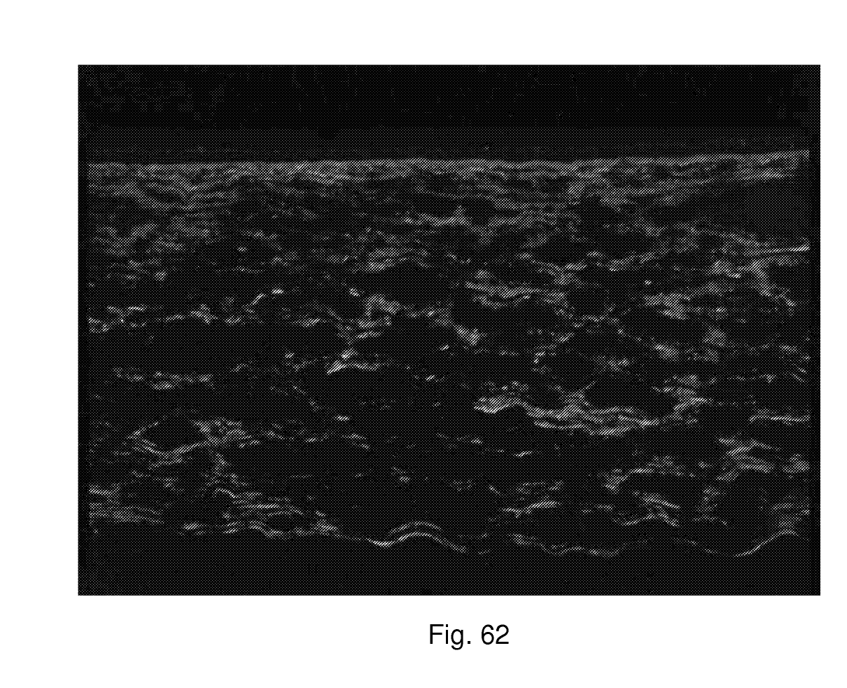

Lipid deposits have been reported particularly in the superficial stroma and Bowman's. These stain positive with oil red O or Sudan black. But these dyes are lipid-soluble and stain only esterified cholesterol, not unesterified cholesterol (Rodrigues, et al., Am J Opthalmol 1990; 110:513-517) (FIG. 61). Nonesterified cholesterol, cholesterol esters, and phospholipids have been found to be the predominant lipids in the SCCD cornea. Crystalline deposits in SCCD have been shown to be cholesterol. (Garner, et al., Br J Opthalmol 1972; 56:400-408; Delleman, et al., Opthalmologica 1968; 155:409-426; Bonnet, et al., Bull Soc Ophtalmol Fr 1934; 46:225-229; Rodrigues, et al., Am J Opthalmol 1990; 110:513-517).

The typical compounds that are used for ultrastructural studies, such as osmium tetroxide and organic solvents and resins, can dissolve lipids. However, cryoultramicroscopy allows ultra-thin sections of cryopreserved lipid-laden tissue that can then be stained with filipin, which is a fluorescent probe that specifically detects unesterified cholesterol (FIG. 62).

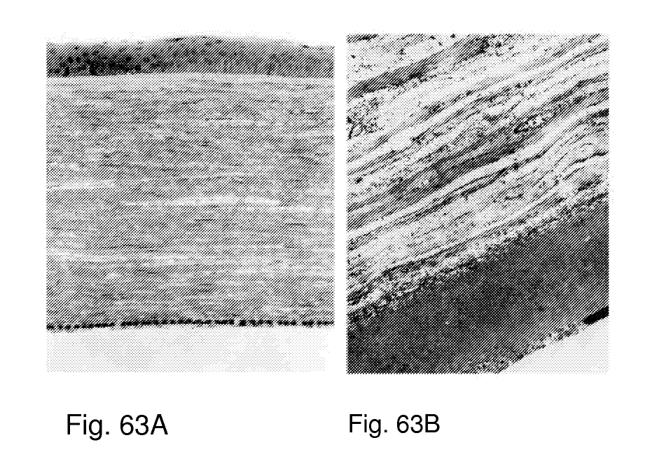

This technique reveals that the major constituent of the corneal deposit in SCCD is unesterified cholesterol with smaller amounts of other lipids. (Lisch, Klin Monatsbl Augenheilkd 1977; 171:684-704). Electron microscopic analysis has revealed intracellular and extracellular lipid throughout the stroma with vacuoles representing dissolved lipid cholesterol in the basal epithelium, stroma, and occasionally within endothelial cells (FIGS. 63A-B). (Weiss, et al., Opthalmology. 1992; 99:1072-1081).

Animal models for SCCD exist. Histopathology of the condition in the animal mode is similar to that found in humans. (Crispin, et al., J Small Anim Pract 1983; 24:63-83; Crispin, et al., Clin Sci 1988; 74:12). Crystalline stromal dystrophy is the commonest canine corneal lipid deposition and is relatively common in the Cavalier King Charles Spaniel. Corneal opacities similar to SCCD have also been produced by feeding a cholestanol-enriched diet to BALB/c mice, but these are associated with corneal vascularization, which is not present in SCCD. In this animal model, the serum cholestanol was 30 to 40 times normal, and the corneal deposits were composed of calcium phosphorous and probably cholestanol (Kim K S, et al., Biochim Biophys Acta 1991; 1085:343-349).

2. Chemical Analysis

Quantitative analysis of the cornea in SCCD reveals that the lipid accumulation is mostly unesterified cholesterol and phospholipids. (McCarthy, et al., Opthalmology 1994; 101:895-901). Lipid analysis of the corneal specimens from patients affected with SCCD who have undergone PKP demonstrates that apolipoprotein constituents of HDL (apo A-I, A-II, and E) are accumulated in the central cornea, whereas those of the LDL (apo B) are absent. This suggests an abnormality confined to HDL metabolism. HDL concentrations in the serum are inversely related to the incidence of coronary atherosclerosis. (Murray, et al., Harpers Biochemistry: Cholesterol Synthesis, Transport and Excretion 2005; 26).

Chemical analysis of corneas removed from patients with SCCD reveal that the cholesterol and phospholipids contents increase greater than 10-fold and 5-fold, respectively, in affected corneas compared to normal corneas. Sixty-five percent of the cholesterol is unesterified compared to the control cornea, where 50% is esterified. Unesterified cholesterol to phospholipid molar ratios (1.5 vs. 0.5) are higher in affected compared with normal corneas. Western blots confirm increased amounts of HDL apolipoproteins, indicating that there is a specific local metabolic defect in HDL metabolism in the corneas of SCCD patients. Interestingly, human and animal atherosclerotic lesions have also been reported to stain positive for filipin, demonstrating the accumulation of unesterified cholesterol. (DiFerdinando, G Ital Oftalmol 1954; 7:476-484; Gaynor, et al., Arterioscler Thromb Vasc Biol 1996; 16:993-999; Kruth, Atherosclerosis 1987; 63:1-6)

Yamada and associates (Yamada, et al., Br J Opthalmol 1998; 82:444-447) confirmed the findings of increased unesterified cholesterol in the SCCD cornea with their chemical analysis that the SCCD cornea had only 14% of cholesterol esterified in comparison 60% to 71% esterified corneal cholesterol found in controls. Sphingomyelin was found at 33 times the concentration that was found in controls. Primary lipid keratopathy is also reported to have elevated unesterified cholesterol and sphingomyelin.

3. Similarity to Findings in Atherosclerosis

Filipin-stained deposits of unesterified cholesterol that are found in the SCCD cornea are similar to the filipin-stained deposits of unesterified cholesterol found in atherosclerotic lesions. In the vessels, plasma lipoprotein is the source of cholesterol. It is unclear what the source of cholesterol is in the SCCD cornea (Kruth, Atherosclerosis 1987; 63:1-6).

### H. Additional Characteristic Corneal Findings In SCCD

### Corneal Sensation

While many patients did not have assessment of corneal sensation; approximately 27 of 43 (63%) of eyes of patients ≧40 years of age had decreased corneal sensation. In patients ≧40 years of age, 3 of 7 eyes had decreased corneal sensation in pedigree A, 6 of 12 (50%) in pedigree B, and 19 of 35 (54%) in patients examined by the author. While pooling of objective measurements of corneal sensation like Cochet Bonnet, with subjective assessment of the cotton wisp test, was not ideal for statistical analysis; the studies funding is confirmed by previous published reports of decreased corneal sensation in SCCD. (Brownstein, et al., Can J Opthalmol 1991; 26:273-279; Grop, Acta Opthalmol Suppl (Copenh) 1973; 12:52-57; Ehlers, et al., Acta Opthalmol (Copenh) 1973; 51:316-324).

Confocal microscopy has demonstrated the deposition of highly reflective deposits in the early stages of SCCD. Lipid deposits are noted inside keratocytes and along basal epithelial/subepithelial nerve fibers. Later in the disease, deposition of large extracellular crystals and reflective extracellular matrix results in disruption of basal epithelial/subepithelial nerve plexus. This corresponds with the clinical finding of loss of corneal sensation. (Ciancaglini, et al., J Cataract Refract Surg 2001; 27:1892-1895; Vesaluoma, et al., Opthalmology 1999; 106:944-951).

### I. Visual Loss In SCCD

### Scotopic Versus Photopic Visual Acuity in the SCCD Patient

The literature has suggested that SCCD typically causes minimal visual morbidity, with some investigators even reporting that “visual acuity often is unaffected,” (Ingraham, et al., Opthalmology 1993; 100:1824-1827). For purpose of statistical analysis, both UCVA and BCVA were converted to logMAR units for all analysis in this study.

To assess the actual impact of SCCD on visual acuity, a 3-pronged approach was taken. The first was determining the visual acuity on initial examination of all patients who had no other ocular pathology and plotting the BCVA with increasing patient age (FIG. 37).

The second approach was to determine how vision had changed in the individual patient with time (Table 4).

The third approach was to examine the number of patients who reported corneal surgical intervention. The BCVA within 1 year prior to PKP was examined to determine the indications for intervention (Table 5). The percentage of patients in each decade of age that had reported undergoing PTK or PKP was also graphed (FIG. 55). Surgical intervention was assumed to be an indirect indication of visual loss, as presumably only those patients with significant visual disability would undergo PKP or PTK.

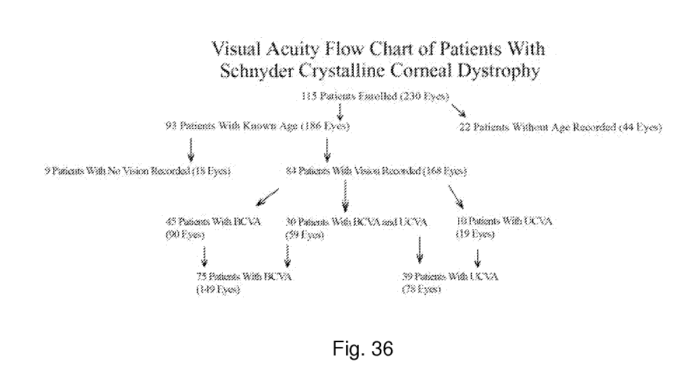

While 75 of 93 patients had BCVA on initial examination (FIG. 36); 44 of these 149 eyes were eliminated from analysis because of coexisting ocular pathology, including prior corneal surgery, cataracts, amblyopia, macular degeneration, and other retinal pathology. Perhaps somewhat predictably, 38 of the eyes with coexisting ocular pathology were in patients ≧40 years of age with the most frequent exclusionary factor being cataract. Although it is possible that some of the cataracts were visually insignificant and perhaps these eyes did not have to be excluded from visual acuity analysis, stringent criteria gave more assurance that any visual decrease associated with age would most likely only be associated with increasing corneal opacification because of SCCD.

While there was a statistically significant decrease in BCVA between those patients ≧40 years and those <40 (P<0.0001), the mean Snellen BCVA was excellent in all age-groups. In those patients <40 years of age, mean Snellen BCVA was between 20/20 and 20/25, and in those patients ≧40 years of age, mean Snellen BCVA was between 20/25 and 20/30. Regression analysis demonstrated a weak trend of small deterioration in BCVA with age (FIG. 37).

The overall maintenance of good visual acuity and the slow deterioration of BCVA were confirmed in the small cohort of 34 eyes that had 7 or more years of follow-up with a mean follow-up of 11.4 years. While 7 of 34 eyes underwent PKP, 21 eyes stayed within I line of initial visual acuity. Four additional eyes lost 2 lines of BCVA. Two eyes lost 3 lines of BCVA to final BCVA of 20/40 OU. All other eyes which had no other concomitant pathology had a final BCVA of at least 20/30. In fact, a 61-year-old woman from Family D who had been followed for 15 years maintained a BCVA OU of 20/25 on her most recent visit (Table 4).

Lisch and associates (Lisch, et al., Ophthalmic Paediatr Genet. 1986; 7:45-56) reported on 13 patients affected with SCCD that were followed for 9 years. All patients who were less than 40 years of age maintained visual acuity of at least 20/30 on second examination. Of the 3 patients that were 40 years or older, a 68-year-old had PKP, with preoperative visual acuity of 20/80 but no mention was made if there was any other ocular pathology; another 65-year-old maintained 20/30 visual acuity; and a 48-year-old had visual decrease from 20/50 OU to 20/100 OU. Unfortunately, no information was provided as to other ocular pathology, such as cataract formation.

In the current study, the slow deterioration of visual acuity and the maintenance of excellent BCVA did not explain why such a large percentage of eyes (7 of 34, 21%) followed for at least 7 years had PKP. Apparently, there was a visual impairment that was not explained by the measurement of scotopic visual acuity alone. Glare testing was not included in initial protocol and was documented in only a few patients older than 40, so the percentage of patients having loss of photopic vision could not be quantified.

However, some charts did indicate that there was a subjective complaint of glare and a marked decrease in vision in the lightened room for some patients. The difference between scotopic and photopic visual acuity in the SCCD patient was discussed by Paparo and coworkers, (Paparo, et al., Cornea 2000; 19:343-347) who postulated that diffraction of light from corneal crystals resulted in a loss of photopic vision in SCCD. Fagerholm (Fagerholm, Acta Opthalmol Scand 2003; 81:19-32) further suggested that although the crystals could result in light diffraction causing glare and photophobia, the diffuse general haze itself was another cause of decreased vision.

An attempt to quantify the effect of SCCD on photopic vision was performed over a decade ago by Van den Berg and coworkers. (Van den Berg, et al., Doc Opthalmol 1993; 85:13-19).

They postulated that the phenomenon of intraocular straylight explained the reduced visual quality in SCCD. Intraocular straylight occurs “when the retina receives light at locations that do not optically correspond to the direction the light is coming from.” Straylight was increased in the 4 eyes of SCCD patients that they measured, while visual acuity was relatively spared. This light-scattering phenomenon explained why patients were frequently bothered by loss of contrast and glare. The investigators thought that the corneal opacification, rather than the crystals alone, were the cause of the abnormal light scattering, which resulted in decreased visual quality, retinal contrast reduction, and glare. In a darkened room, they noted the patient maintained “relatively well preserved visual acuity.” (Van den Berg, et al., Doc Opthalmol 1993; 85:13-19).

The stray light hypothesis suggested a reason for the higher numbers of PKPS in the long-term follow-up of SCCD patients in this study than would have been anticipated considering the benign visual prognosis that this dystrophy has traditionally carried. Although the level of visual deterioration was slow and good BCVA seemed to be maintained; an increasing percentage of patients still underwent PKP with age. BCVA was reported to be as good as 20/25 in one patient prior to PKP. At the same time, those few patients who had glare testing documented demonstrated a decrease in visual acuity when lights were turned on.

### J. Prevalence Of PKP In SCCD

Although there are frequent reports of PKP in SCCD (Lisch, et al., Ophthalmic Paediatr Genet. 1986; 7:45-56; Yamada, et al., Br J Opthalmol 1998; 82:444-447; Freddo, et al., Cornea 1989; 8:170-177; Hoang-Xuan, et al., J Fr Ophtalmol 1985; 8:735-742; Rodrigues, et al., Am J Opthalmol 1987; 104:157-163; Weller, et al., Br J Opthalmol 1980; 64:46-52; Delleman, et al., Opthalmologica 1968; 155:409-426; Ehlers, et al., Acta Opthalmol (Copenh) 1973; 51:316-324; Pfannkuch, Klin Monatsbl Augenheilkd 1978; 173:355-358; Rodrigues, et al., Am Opthalmol 1990; 110:513-517; Eiferman, et al., Metab Pediatr Syst Opthalmol. 1979; 3:15) the literature reports that SCCD “rarely requires corneal grafting.” (Weller, et al., Br J Opthalmol 1980; 64:46-52; Gillespie, et al., Am J Opthalmol 1963; 56:465-467).

In the current study, 39 eyes of 27 patients underwent PKP with an increasing number of PKPs reported as patients aged. The prevalence of PKP in patients ≧50 years was 20 of 37 (54%). Ten of 13 patients ≧70 years (77%) had PKP. Only 3 patients ≧70 had no history of having PKP. Chart notes of the 2 older patients who had not had corneal surgery indicated that PKP was being considered. Chart notes were unavailable for the third patient, who lived in Turkey. This analysis implied that PKP was either performed or strongly considered in every SCCD patient who was above the age of 70.

Why was PKP performed so frequently if the BCVA did not appear to be markedly decreased? The first possibility was that selection bias recruited patients with more severe disease and artificially resulted in an increased PKP prevalence in this disease. This possibility was previously discussed in section II, E above. A second possible explanation for the large number of PKPs performed was that PKPs could have been performed earlier than usual if the corneal surgeon was more aggressive. However, each of the patients who had preoperative BCVA of 20/50 or better within 1 year prior to the PKP originated from a different pedigree and had the PKP performed by a different surgeon. Another possibility for a higher surgical intervention than anticipated was that the approach to SCCD has changed during the years with earlier intervention because of the successful results of PKP surgery. While any of these explanations could explain a higher number of PKPs than would be expected on the basis of the corneal findings and visual acuity, the analysis of the individual pedigrees that had excellent follow-up still serves to give a good estimate of PKP frequency.

### K. Preoperative Visual Acuity and Glare Before PKP

Although the study was limited by number of patients who had preoperative vision within 1 year of PKP, 13 eyes had preoperative BCVA within 1 year of PKP documented.

Nine eyes of 5 patients had preoperative BCVA that was ≧20/50, including one eye with cataract and another with prior PTK. Only 3 patients with preoperative BCVA ≧20/50 had no concomitant ocular pathology. However, all 3 had preoperative documentation of glare complaints or decrease in vision under photopic conditions. The combination of good BCVA prior to surgery with a documentation of a subjective complaint of glare supports the hypothesis that SCCD can disproportionately affect scotopic vision and motivate the patient to have PKP sooner than the photopic vision might indicated.

The question of subjective glare was further clarified by an attempt to repeat the phone interview of the 55 American patients who had originally responded to phone or written follow up. Forty-one patients were reached and again interviewed by phone. Patients were asked about symptoms of glare during day and night and about functional limitations such as difficulty reading, using a computer, driving during day or night because of visual problems. (Shildkrot™, et al., Poster presented at: Association for Research in Vision and Opthalmology meeting in Fort Lauderdale, Fla., 2007)

Mean patient age was 43.8±21.0 years (range, 6-83 years). Subjective decrease in near and distance vision was reported by 6 of 41 patients (14.6%) Nighttime glare was reported by 26 of 41 patients (63.4%), of whom 9 stopped or limited night driving. Nighttime glare was reported in 0 of 8 patients <25 years of age, 10 of 12 patients (83.3%)≧25 and <45 years of age, and 16 of 21 patients (76.2%)≧45 years of age. Daytime glare was reported by 11 of 41 patients (26.8%), one of whom reported having to stop watching television because of glare problems. Daytime glare was reported in 0 of 8 of patients <25 years of age, 1 of 12 (8.3%) patients ≧25 and <45 years of age, and 10 of 21 patients (47.6%)≧45 years of age. Prevalence of reported glare increased with age both in daytime (P=0.008) and nighttime (P=0.0002).

The brief phone survey had many limitations, including providing subjective, not objective, information about the prevalence of glare and lack of a control group to compare the prevalence of glare to a population unaffected with SCCD. However, the data still provides some confirmation that glare appears to be a prominent complaint in patients with SCCD and that the complaint of glare increases with age. This lends support to the hypothesis of Van den Berg and coworkers (Van den Berg, et al., Doc Opthalmol 1993; 85:13-19) that progressive corneal opacification in SCCD causes light scattering. In addition, this would support the hypothesis that glare symptoms could be a potential cause for the high number of PKP in the SCCD population.

### L. Indications For PKP In The Literature For SCCD And Other Stromal Dystrophies

Most articles written about PKP in SCCD are case reports, and so there is no recommendation in the literature on when to perform PKP for the SCCD patient. In addition, case reports on PKP in SCCD often lack important data to assess indications for surgery. For example, Weller and Rodger reported PKP was performed for “unmarried woman in her 50s . . . who couldn't carry out her job” but the authors did not list vision prior to PKP. (Weller, et al., Br J Opthalmol 1980; 64:46-52).

Ingraham (Ingraham, et al., Opthalmology 1993; 100:1824-1827) reported PKP in a 46-year-old with BCVA of 20/80 but did not indicate whether there was any other pathology that could be causing visual decrease, such as cataract. Rodrigues, et al., discussed PKP OD for a 57-year-old with BCVA OD of count fingers and OS 20/50 and complaints of photophobia but the patient also had cataract formation more prominent in the OD than OS. (Rodrigues, et al., Am J Opthalmol 1990; 110:513-517). Was the SCCD causing the visual decrease and photophobia OD, or was it the cataract? The aging patient can have concomitant ocular pathology, such as cataract formation, which can reduce vision and cause glare symptoms. Without clear information about the complete ocular examination, it is difficult to use the published literature to clearly determine the indications for surgical intervention in SCCD.

How does the preoperative level of BCVA in the patients in this report prior to PKP compare to 2 studies of patients with corneal stromal dystrophies undergoing PKP? Ellies and coworkers examined 110 eyes of 73 patients with BIGH3 mutations who underwent PKP. (Ellies, et al., Opthalmology. 2002; 109:793-797). The investigators indicated that PKP was performed for BCVA that was 20/80 or worse. Another study, by Al-Swailem and coworkers, reports 229 PKPs that were performed in patients with macular dystrophy; 68% of patients had preoperative visual acuity of 20/100 to 20/180.

1. Success Of PKP In SCCD

The present study was limited by the lack of information on preoperative vision within a year of surgery and postoperative vision in the majority of PKP eyes. The 11 eyes in with documentation of both preoperative and postoperative visual acuity appeared to do well after PKP. Five eyes improved by 1 or more lines of BCVA. One eye with 20/30 BCVA preoperatively maintained the same visual acuity postoperatively. The remaining 5 eyes had other ocular diagnoses, including suture abscess or macular degeneration, and maintained the same visual acuity or loss of I line of vision. Only 1 patient reported a graft rejection and no patients reported repeat PKP in the same eye.

2. PTK

PTK has been reported to be successful in removing crystalline opacities that are impairing vision in SCCD. (Paparo, et al., Cornea 2000; 19:343-347; Ciancaglini, et al., J Cataract Refract Surg 2001; 27:1892-1895; Fagerholm, Acta Opthalmol Scand 2003; 81:19-32; Herring, et al., J Refract Surg 1999; 15:489; Koksal, et al., Cornea 2004; 23:311-313; Forster, et al., Graefes Arch Clin Exp Opthalmol 1997; 235:296-305; Maloney, et al., Am J Opthalmol 1996; 122:149-160; Orndahl, et al., J Refract Surg 1998; 14:129-135; Rapuano, Cornea 1997; 16:151-157; Rapuano, et al., CLAO J 1993; 19:235-240; Rapuano, et al., CLAO J 1994; 20:253-257; Tuunanen, et al., CLAO J 1995; 21:67-72). Researchers have reported 4 eyes of 3 patients with SCCD and central corneal crystals who had PTK. (Paparo, et al., Cornea 2000; 19:343-347) In all cases, the patients complained of glare or photophobia, and BCVA worsened in the lighted room. When crystals were removed after PTK, there was subjective improvement in glare and photophobia and average BCVA improved from 20/175 to 20/40 in bright light, but vision was still best under scotopic conditions. However, the average hyperopic shift was +3.28.

In the present study, PTK was performed to remove the central cholesterol crystals that were causing impairment of vision. Three patients underwent PTK with an improvement in vision in 4 of 5 eyes. PTK in one eye of a 41-year-old patient did not improve the preoperative BCVA of 20/50, and the patient subsequently had PKP (FIG. 52B). This patient was older than the other 2 patients who had successful PTK. By age 41, it was possible that concomitant stromal opacification resulted in visual decrease even after the crystalline opacity was removed by PTK.

3. Recurrence

Recurrences of SCCD after PKP have been previously reported, (Brownstein, et al., Can J Opthalmol 1991; 26:273-279; Lisch, et al., Ophthalmic Paediatr Genet. 1986; 7:45-56; Garner, et al., Br J Opthalmol 1972; 56:400-408; Delleman, et al., Opthalmologica 1968; 155:409-426) but there is no consensus how frequently this occurs. Delleman and Winkelman indicated recurrence was common. (Delleman, et al., Opthalmologica 1968; 155:409-426). In a retrospective review of all patients with stromal dystrophies undergoing PKP at Wills Eye Hospital between 1984 and 2001, only 4 eyes of 4 patients with SCCD had PKP. There was no recurrence of the dystrophy in any of the eyes in up to 4.6 years of follow-up and so the investigators concluded that the dystrophy had a low recurrence rate. This compared to a follow-up of 5 years with a recurrence rate of 88% in corneal dystrophies of Bowman's layer, 40% recurrence rate in granular dystrophy, and a 17.8% recurrence rate in lattice dystrophy. (Marcon, et al., Cornea 2003; 22:19-21).

In this study, 5 of the 27 patients and 8 of the 39 eyes (21%) undergoing PKP had evidence of recurrence. While all of these patients had bilateral PKP, recurrence was unilateral in 2 patients and bilateral in 3 patients. The rate of recurrence for SCCD in this study appears to be most similar to the recurrence rate for lattice dystrophy found by Marcon and associates. (Marcon, et al., Cornea 2003; 22:19-21).

### M. Differential Diagnosis Of SCCD

1. Crystalline Deposits, Cloudy Corneas, and Disorders of Lipid Processing

Crystalline deposits can be found in numerous diseases, including cystinosis, dysproteinemias, multiple myeloma, monoclonal gammapathy, calcium deposits, oxalosis, hyperuricemia, Tangier disease, tyrosinosis, porphyria, Bietti's crystalline dystrophy, infectious crystalline keratopathy; instillation of sap from the Dieffenbachia plant; and in association with ingestion of drugs such as gold, indomethacin, chlorpromazine, chloroquine, and clofazimine. (Brownstein, et al., Can J Opthalmol 1991; 26:273-279; Brooks, et al., Opthalmology 1988; 95:448-452).

Primary or secondary lipid corneal degeneration is associated with corneal neovascularization with subsequent leakage of lipid into the Cornea While primary lipid corneal degeneration has no known underlying cause, secondary lipid degeneration is typically secondary to chronic inflammation. In both entities, progressive lipid deposition results in corneal opacification with potential decrease in visual acuity. Histopathology reveals lipid granules, histiocytes, vascularization, and nongranulomatous inflammation. (Baum, Am J Opthalmol 1969; 67:372-375; Spraul, et al., Klin Monatsbl Augenheilkd 2002; 219:889-891).

This is easily distinguished from SCCD because corneal blood vessels are absent in SCCD. (Duran J A, et al., Cornea 1991; 10:166-169). Familial lecithin-cholesterol acyltransferase deficiency (LCAT), fish eye disease, and Tangier disease should also be considered in the differential diagnosis of SCCD. (Bron, Cornea 1989; 8:135-140; McIntyre, J Inherit Metab Dis 1988; 11(Suppl 1):45-46).

2. LCAT

In LCAT, there is absence of the LCAT enzyme that is involved in cholesterol metabolism. Unlike SCCD, LCAT is inherited in an autosomal recessive mode with deficient activity of the enzyme LCAT to esterify cholesterol in the LDL and HDL particles. The plasma can appear turbid because of the elevated free cholesterol and lecithin levels. Normochromic anemia and/or renal disease can occur.

Similarly to SCCD, corneal changes can occur before puberty with a prominent arcus lipoides and minute gray diets affecting the entire corneal stroma. (Vrabec, et al., Arch Opthalmol 1988; 106:225-229). When crystals occur, they occur in the peripheral stroma near Descemet's rather than the superficial stroma like SCCD. Vacuoles are noted in Bowman's layer and throughout the stroma. (Bethell, et al., Can J Opthalmol 1975; 10:494-501).

3. Fish Eye Disease

In the extremely rare disease fish eye, the LCAT enzyme has deficient activity in esterifying cholesterol in HDL particles (McIntyre, J Inherit Metab Dis 1988; 11(Suppl 1):45-46). The disease is autosomal recessive with little systemic disorder except for hypertriglyceridemia and reduced HDL levels. On clinical examination of the patient with fish eye disease, there is almost complete corneal opacification, sometimes with arcus noted and significant loss of vision by age 15. Phospholipid and cholesterol are noted throughout the corneal layers except epithelium on histopathology examination.

4. Tangier Disease

Tangier disease results from a deficiency of HDL and apolipoprotein, apo A1, due to increased catabolism. Many associated systemic disorders can accompany this autosomal recessively inherited disease, including lymph node enlargement, peripheral neuropathy, and hepatosplenomegaly. No arcus lipoides is noted, although there is a granular stromal haze. LCAT activity is normal, triglycerides are elevated, and there is a reduction of total cholesterol, HDL and LDL. (Schaefer, et al., Ann Intern Med 1980; 93:261-266).

Although all these diseases affect cholesterol metabolism and cause corneal clouding, there are many characteristics that allow differentiation from SCCD. Whereas SCCD is inherited in an autosomal dominant mode, LCAT, fish eye, and Tangier are autosomal recessive inherited diseases. None of the diseases have the subepithelial cholesterol crystalline deposition that can occur in SCCD. HDL is not typically affected in SCCD, but low HDL levels are seen in LCAT, fish eye and Tangier disease. (Weiss, et al., Opthalmology. 1992; 99:1072-1081).

### N. Pathogenesis

1. Hyperlipidemia and Corneal Clouding in SCCD—Independent Variables or Causative Association and the Role of UBIAD1 in Understanding Disease Mechanism

While premature occurrence of corneal arcus is reported to be associated with coronary artery disease, (Halfon, et al., Br J Opthalmol 1984; 68:603-604; Rouhiainen, et al., Cornea 1993; 12:142-145; Virchow, Virchow's Arch Path Anat. 1852; 4:261-372) corneal arcus has also been reported to occur independent of abnormal lipid levels or other systemic disorders. (Barchesi, et al., Sury Opthalmol 1991; 36:1-22). Previously, the systemic hyperlipidemia in SCCD was postulated to be the primary defect resulting in corneal clouding, (Sysi, Br J Opthalmol 1950; 34:369-374; Bron, et al., Br J Opthalmol 1972; 56:383-399; Bonnet, et al., Bull Soc Ophtalmol Fr 1934; 46:225-229) but this theory lost favor when others documented that patients affected with SCCD can have either normal or abnormal serum lipid, lipoprotein, or cholesterol levels. (Barchesi, et al., Sury Opthalmol 1991; 36:1-22; Bron, et al., Br J Opthalmol 1972; 56:383-399; Rouhiainen, et al., Cornea 1993; 12:142-145).

Although familial hypertriglyceridemia and dysbetalipoproteinemia have been reported, familial hypercholesterolemia is the most common lipoprotein abnormality found (Kajinami, et al., Nippon Naika Gakkai Zasshi 1988; 77:1017-1020; Thiel, et al., Klin Monatsbl Augenheilkd 1977; 171:678-684; Crispin, Prog Retin Eye Res 2002; 21:169-224) in patients with SCCD. Hypercholesterolemia has been reported in up to two-thirds of patients with SCCD. (Sverak, et al., Cesk Oftalmol 1969; 25:283-287 Karseras, et al., Br J Opthalmol 1970; 54:659-662; Williams, et al., Trans Opthalmol Soc UK 1971; 91:531-541). By comparison, the Cavalier King Charles Spaniel and rough collie breeds of dog with crystalline dystrophy usually have normal serum lipid levels. (Crispin, Cornea 1988; 7:149-161).

Lisch and associates (Lisch, et al., Ophthalmic Paediatr Genet. 1986; 7:45-56) followed 13 patients with SCCD for 9 years and concluded that no link could be drawn between the corneal findings and systemic hyperlipidemia, although 8 of 12 patients had elevated cholesterol or apolipoprotein B levels and 6 of 8 had dislipoproteinemia type IIa. Consequently, it is likely that the gene for SCCD results in an imbalance in local factors affecting lipid/cholesterol transport or metabolism. A temperature-dependent enzyme defect has been postulated because the initial cholesterol deposition occurs in the axial/paraxial cornea, which is the coolest part of the cornea. (Crispin, Prog Retin Eye Res 2002; 21:169-224).

Plasminogen activator secretion was also reported as being decreased in SCCD corneal fibroblasts when compared to normal fibroblasts, but this work has not been reduplicated. (Mirshahi, et al., C R Acad Sci III 1990; 311:253-260). The possibility that the gene for SCCD plays an important role in lipid/lipoprotein metabolism throughout the body is supported by an article by Battisti and coworkers, (Battisti, et al., Am J Med Genet. 1998; 75:35-39) who cultured the skin fibroblasts obtained from a skin biopsy of a patient with SCCD. Membrane-bound spherical vacuoles with lipid materials suggesting storage lipids were present in the skin. This work has not been reproduced.

Work by Burns and associates (Burns, et al., Trans Am Opthalmol Soc. 1978; 76:184-196) documented the cornea as an active uptake and storage site for cholesterol. They injected radioactively labeled 14C-cholesterol 11 days prior to removing a patient's cornea during PKP and demonstrated that the level of radioactive cholesterol was higher in the cornea than the serum at the time of surgery. Furthermore, lipid analysis of the corneal specimens from patients affected with SCCD who have undergone PKP revealed that the apolipoprotein constituents of HDL (apo AI, A-II and E) were accumulated in the central cornea, while those of the LDL (apo B) were absent. This suggested an abnormality confined to HDL metabolism. (Gaynor, et al., Arterioscler Thromb Vasc Biol 1996; 16:993-999). Because of its smaller size, HDL would be the only lipoprotein that could freely diffuse while intact to the central Cornea The size of the larger lipoproteins would prevent their free diffusion unless they were modified (Bron, Cornea 1989; 8:135-140).

HDL concentrations are inversely related to the incidence of coronary atherosclerosis. (Murray, et al., Harpers Biochemistry: Cholesterol Synthesis, Transport and Excretion 2005; 26). Consequently, it appears that SCCD is directly related to a local defect of HDL metabolism, but the relevance of abnormal HDL corneal metabolism is not yet established.

Recent discovery of UBIAD1 as the causative gene for SCCD will provide the mechanism to understand the pathogenesis of this disease. UBIAD1 contains a prenyltransferase domain that could play a role in cholesterol metabolism. Prenylation reactions are involved in cholesterol synthesis, and it is possible that excess cholesterol synthesis results from a defective gene. In addition, UBIAD1 interacts with the C-terminal portion of apo E which is known to be important in reverse cholesterol transport. Consequently, another possible disease mechanism could be that decreased cholesterol removal from the cell results from an alteration in the interaction with apo E. (Weiss, et al., Invest Opthalmol Vis Sci 2007; 48:5007-5012).

Although this study was not meant to examine cholesterol issues exhaustively, patients who had PKP were asked whether or not they had hypercholesterolemia and if they were on cholesterol-lowering medication. Twenty-one of the 29 patients who had corneal surgery lived in the United States, and 5 of these were deceased. Of the remaining 16 patients, 14 were contacted by telephone.

While 12 of the 14 patients (86%) reported elevated cholesterol levels, 4 of the 14 (29%) had a history of cardiac disease and 10 of the 14 (71%) were on a cholesterol-lowering agent. The mean age of patients with hypercholesterolemia was 68±10.5 years (range, 52-82). There was no statistical difference between the percentage of patients who were ≧50 and who were on cholesterol-lowering medications among patients who had corneal surgery compared to those who did not have corneal surgery (P=0.34). The few studies on the effect of systemic cholesterol on progression of the dystrophy conclude that these are independent traits, (Lisch, et al., Ophthalmic Paediatr Genet. 1986; 7:45-56) but the numbers of patients and length of follow-up are too small to draw any definitive conclusions. None of the previously published studies have looked at cholesterol measurements specifically in an older cohort.

2. Coronary Artery Disease and Myocardial Infarction

Although the purpose of this study was to assess the visual morbidity of SCCD, the frequency of hypercholesterolemia in the PKP patient presented the question of whether or not there was early mortality from cardiovascular disease.

Four patients who had PKP and were on cholesterol-lowering medication reported coronary artery disease or prior myocardial infarction. The age at death and cause of mortality for the 8 patients who were known to die during the study were also assessed. Four patients died in the 9th decade. One of these patients had a history of myocardial infarction and the other congestive heart failure. Three brothers died before the 6th decade; one from brain cancer and the other two from auto accidents. Only one patient who died before the 7th decade had a cardiac-related diagnosis of coronary artery disease, bacterial endocarditis, and sepsis.

Although the study is too small to detect any increased risk of mortality from cardiovascular events in this population, it is reassuring that 7 of the 8 deaths did not appear to be a result of premature death from cardiovascular disease.

The importance of obtaining cholesterol measurements in the affected and unaffected members of SCCD pedigrees has been previously emphasized in the literature. (Kohnen, et al., Klin Monatsbl Augenheilkd 1997; 211:135-136). Perhaps the apparent infrequency of cardiac mortality in this cohort, combined with the large numbers of patients (Gillespie, et al., Am J Opthalmol 1963; 56:465-467) undergoing corneal surgery ≧50 years who are taking cholesterol-lowering agents; underscores that appropriate diagnosis and treatment are successful interventions in this disease.

3. Genu Valgum

Genu valgum has been postulated to be an independent trait (Brownstein, et al., Can J Opthalmol 1991; 26:273-279; Barchesi, et al., Sury Opthalmol 1991; 36:1-22; Hoang-Xuan, et al., J Fr Ophtalmol 1985; 8:735-747) reported in association with SCCD. The percentage of patients with SCCD that have this finding is not known, but Delleman and Winkelman (Delleman, et al., Opthalmologica 1968; 155:409-426) reported that 16 of the 21 SCCD patients in a 6-generation pedigree had genu valgum. Only 1 of 33 patients with SCCD had genu valgum (Hoang Xuan T, et al., J Fr Ophtalmol 1985; 8:743-747) in the 4 Swede-Finn pedigrees previously reported. In the current study, 5 patients in three families had genu valgum.

SCCD has previously been a poorly understood disease because of its rarity and spectrum of clinical manifestations. The present study represents the largest number of patients with SCCD and the longest follow-up of patients with SCCD ever published. The information obtained from this large case series should clarify both the clinical findings and the course of SCCD.

The ophthalmologist must be aware that despite individual variations, there are predictable changes in the corneal opacification pattern that can occur with age and that the characteristic crystals may not always be seen on examination. The pathologist must be made aware prior to processing the corneal specimen that SCCD is a consideration so that the cornea be placed in fixatives that will not dissolve lipid and prevent pathologic diagnosis.

A goal of this example was to attempt to answer the most frequent question asked by a patient newly diagnosed with SCCD. “What can I expect to happen with time?” The patient can be reassured that scotopic vision can be excellent into their 5th decade and beyond. It is most likely that that the major visual disability experienced is loss of photopic vision. In this study, surgical intervention occurred in 54% of patients 50 years and above and almost 77% of patients in the 8th or 9th decade.

Another, perhaps unasked, question is the impact of systemic hypercholesterolemia on mortality. It was reassuring to discover that only 1 of the 8 deaths might have been associated with premature demise from cardiovascular disease. The majority of the nonaccidental deaths were patients in their 9th decade. Consequently, the proper concomitant monitoring and treatment of systemic hyperlipidemia is imperative and could have resulted in normal life span in the majority of patients studied.

TABLE 2
DEMOGRAPHY AND SURGERY IN SCHNYDER CRYSTALLINE
CORNEAL DYSTROPHY PEDIGREES

| | | | | AVERAGE | | PTS ≧50 WITH | NO. PTS. | NO. PTS. |
|---|---|---|---|---|---|---|---|---|
| FAMILY | MEMBERS | FEMALE | MALE | AGE | SD | SURGERY | PKP | PTK |
| A | 19 | 4 | 15 | 30 | 19 | 2/6 | 2 | 0 |
| B | 18 | 12 | 6 | 35 | 19 | 5/9 | 5 | 1 |
| C | 2 | 2 | 0 | 56 | 23 | 1/2 | 1 | 0 |
| D | 4 | 4 | 0 | 43 | 31 | 1/2 | 1 | 0 |
| E | 3 | 1 | 2 | 22 | NI | 1/1 | 1 | 0 |
| G | 4 | 3 | 1 | 44 | 23 | 1/1 | 1 | 0 |
| H | 1 | 1 | 0 | 23 | NI | 0 | 0 | 0 |
| I | 4 | 0 | 4 | 46 | NI | 0 | 1 | 0 |
| J | 9 | 3 | 6 | 57 | 16 | 3/5 | 3 | 0 |
| K (Germany) | 4 | 2 | 2 | 37 | 14 | 0/1 | 0 | 0 |
| K1 (Germany) | 2 | 1 | 1 | 56 | 15 | 1/1 | 1 | 0 |
| L | 3 | 2 | 1 | 21 | 23 | 0 | 1 | 0 |
| M | 2 | 1 | 1 | 28 | 28 | 0 | 0 | 0 |
| N (Germany) | 2 | 1 | 1 | NI | NI | 0 | 0 | 0 |
| O | 2 | 1 | 1 | NI | NI | 1/1 | 1 | 0 |
| Q | 5 | 3 | 2 | 24 | 13 | 1/1 | 1 | 1 |
| R | 1 | 1 | 0 | 38 | 0 | 0 | 0 | 0 |
| S | 1 | 0 | 1 | NI | NI | 0 | 0 | 0 |
| T | 2 | 1 | 1 | 81 | NI | 0/2 | 0 | 0 |
| U | 1 | 0 | 1 | 44 | 0 | 1/1 | 1 | 0 |
| V | 1 | 1 | 0 | NI | NI | 0 | 0 | 0 |
| W (Turkey) | 5 | 2 | 3 | 51 | 15 | 0/1 | 0 | 1 |
| X (Taiwan) | 1 | 1 | 0 | 38 | NI | 0 | 1 | 0 |
| Y (Germany) | 5 | 3 | 2 | 41 | 18 | 1/1 | 2 | 0 |
| Z | 3 | 2 | 1 | 18 | 18 | 0 | 0 | 0 |
| AA | 1 | 0 | 1 | 63 | NI | 1/1 | 1 | 0 |
| BB (Czech) | 3 | 1 | 2 | 33 | 11 | 0 | 0 | 0 |
| BB1 (England) | 1 | NI | NI | NI | NI | 0 | 1 | 0 |
| BB2 (England) | 1 | NI | NI | NI | NI | 0 | 0 | 0 |
| BB3 (England) | 1 | NI | NI | NI | NI | 0 | 1 | 0 |
| CC (Japan) | 1 | 1 | 0 | NI | NI | 0 | 1 | 0 |
| DD (Taiwan) | 1 | 0 | 1 | NI | NI | 0 | 0 | 0 |
| EE (Taiwan) | 1 | 1 | 0 | 63 | NI | 0/1 | 0 | 0 |
| FF | 1 | 1 | 0 | 42 | NI | 0 | 0 | 0 |
| TOTAL | 115 | 56 | 56 | 39 | 20 | 20/37 | 27 | 3 |

NI, no information; PKP, penetrating keratoplasty; PTK, phototherapeutic keratectomy; Pts, patients; SD, standard deviation.

TABLE 3
CORNEAL SENSATION IN SCHNYDER
CRYSTALLINE CORNEAL DYSTROPHY

| | DECREASED | ≦25 YEARS | 26-39 YEARS | ≧40 YEARS |
|---|---|---|---|---|
| | SENSATION | OF AGE | OF AGE | OF AGE |
| Total | 43/91 (47%) | 10/26 (38%) | 6/22 (27%) | 27/43 (63%) |
| cohort | | | | |
| Author | 29/67 (43%) | 4/12 (33%) | 6/20 (30%) | 19/35 (54%) |
| Family A | 7/20 (35%) | 2/10 | 2/10 | 3/7 |
| Family B | 8/18 (44%) | 2/8 | 0/6 | 6/12 (50%) |

Trans Am Ophthalmol Soc 2007 December; 105: 616-648.

TABLE 4
VISUAL ACUITY WITH LONG-TERM FOLLOW-UP IN PATIENTS
WITH SCHNYDER CRYSTALLINE CORNEAL DYSTROPHY

| PATIENT | | AGE AT | | | AGE AT | | | YEARS | |
|---|---|---|---|---|---|---|---|---|---|
| NUMBER | FAMILY | 1ST EXAM | VA OD | VA OS | 2ND EXAM | VA OD | VA OS | FOLLOW UP | OTHER/PKP |
| II 1 | A | 46 | sc20/25^{‡} | sc20/25^{†} | 58 | sc20/20^{‡} | sc20/30^{†} | 8 | |
| III 1 | A | 23 | sc20/20^{‡} | sc20/20^{‡} | 30 | sc20/15^{‡} | sc20/15^{‡} | 7 | |
| III 7 | A | 19 | sc20/30^{‡} | sc20/25^{‡} | 36 | cc20/25^{‡} | sc20/20^{‡} | 17 | |
| III 2 | B | 14 | cc20/20^{§} | cc20/20* | 21 | cc20/30^{§} | cc20/20* | 7 | |
| III 3 | B | 10 | sc20/30^{‡} | sc20/30^{‡} | 25 | cc20/25^{‡} | sc20/25^{‡} | 15 | |
| II 3 | B | 48 | cc20/30* | cc20/25^{†} | 62 | cc20/30* | cc20/30^{†} | 14 | Cataract |
| III 6 | B | 29 | sc20/20^{¶} | sc20/20^{¶} | 45 | cc20/40^{¶} | cc20/40^{¶} | 16 | Cataract OU |
| 1 | C | 40 | cc20/30§ | cc20/400* | 57 | cc20/50^{§} | cc20/40* | 17 | Amblyopia OS |
| 1 | D | 50 | sc20/25* | sc20/25* | 61 | cc20/25* | cc20/25* | 15 | |
| 2 | D | 32 | sc20/25^{‡} | sc20/20^{§} | 43 | cc20/20^{‡} | cc20/30^{§} | 10 | |
| 1 | G | 60 | cc20/25 | cc20/25 | 67 | PKP | PKP | 7 | PKP |
| | | | | | | Age 61^{#} | Age 62^{#} | | |
| 1 | M | 8 | cc20/25* | cc20/25* | 18 | cc20/25* | cc20/25* | 10 | |
| 1 | Q | 33 | cc20/25^{†} | cc20/25 | 49 | PKP | PKP | 16 | PKP |
| | | | | | | Age 42^{#} | Age 43^{#} | | |
| 2 | Q | 29 | cc20/20^{†} | cc20/20^{†} | 38 | cc20/25^{†} | cc20/25^{†} | 9 | |
| 1 | R | 38 | cc20/20^{§} | cc20/25^{†} | 47 | cc20/30^{§} | cc20/30^{†} | 10 | |
| 1 | U | 44 | cc20/20 | cc20/20 | 54 | PKP | PKP | 9 | PKP |
| | | | | | | Age 45^{#} | Age 52^{#} | | |
| 1 | X | 38 | cc20/70* | cc20/70 | 45 | cc20/70* | PKP | 7 | PKP |
| | | | | | | | Age 38^{#} | | |

cc, with correction; OD, right eye; OS, left eye; PKP, penetrating keratoplasty; sc, without correction; VA, visual acuity.
*Same VA.
^{†}Loss 1 line.
^{‡}Gain 1 line.
^{§}Loss 2 lines.
^{¶}Loss 3 lines.
^{#}PKP eye.

TABLE 5
PREOPERATIVE BEST-CORRECTED VISUAL ACUITY IN
PATIENTS UNDERGOING PENETRATING KERATOPLASTY

| PREOPERATIVE | NO. OF | PATIENT | | AGE at | OCULAR | PHOTOTOPIC |
|---|---|---|---|---|---|---|
| BCVA | EYES | NO. | PEDIGREE | PKP | PATHOLOGY | VISION COMPLAINTS |
| 20/25 | 2 | 1 | G | 61 | No | Lights on BCVA |
| | | | | | | 20/400 |
| | | 1 | G | 62 | No | Lights on BCVA |
| | | | | | | 20/400 |
| 20/30 | 2 | II 9 | A | 47 | No | Glare |
| | | 1 | Q | 43 | No | |
| 20/40 | 1 | 1 | E | 50 | No | Glare |
| 20/50 | 4 | II 9 | A | 51 | No | Glare |
| | | 1 | E | 51 | No | |
| | | 1 | Q | 42 | Prior PTK | |
| | | 1 | AA | 63 | Cataract | |
| 20/70 | 2 | I 1 | B | 64 | Cataract | Lights on BCVA |
| | | | | | | of count fingers |
| | | 1 | X | 38 | No | |
| 20/200 | 1 | 2 | C | 74 | Cataract | Lights on BCVA |
| | | | | | | of count fingers |
| 20/400 | 2 | 2 | C | 72 | Cataract | Lights on BCVA |
| | | | | | | of count fingers |
| | | 3 | D | 76 | SMD | |
| Count fingers | 1 | 3 | D | 81 | SMD | |

BCVA, best-corrected visual acuity; PTK, photherapeutic keratectomy; PKP, penetrating keratoplasty; SMD, senile macular degeneration.

TABLE 6
CHANGE IN VISUAL ACUITY AFTER PENETRATING KERATOPLASTY SURGERY

| | | | INCREASE | | | | | | | | POST- |
|---|---|---|---|---|---|---|---|---|---|---|---|
| | PATIENT | PREOP. | BCVA (LINES) | | | | NO | DECREASE | ADD. | FOLLOW | OPERATIVE |
| PEDIGREE | NUMBER | BCVA | 1 | 2 | 3 | >4 | CHANGE | BCVA (LINES) | SURG.^{†} | UP (YRS) | PATHOLOGY |
| A | II 9 | 20/30 | | | | | X | | | 5 | |
| | II 9 | 20/50 | X | | | | | | | 1 | |
| B | I 1 | 20/70 | | | | | | X | | 4 | Suture Abscess |
| C | 2 | 20/200 | | | | X | | | CE | 5 | |
| | | | | | | | | | IOL | | |
| | 2 | 20/400 | | | | X | | | CE | 4 | |
| | | | | | | | | | IOL | | |
| D | 3 | CF | | | | | X | | CE | 4 | SMD |
| | | | | | | | | | IOL | | |
| G | 1 | 20/25 | | | | | X | | | 6 | Cataract |
| | 1 | 20/25 | | | | | | X | | 7 | Cataract |
| Q | 1 | 20/30 | | | | | | X | | 7 | Cataract |
| | 1 | 20/50 | | X | | | | | | 8 | Cataract |
| X | 1 | 20/70 | | | X | | | | | 7 | |

CE IOL, cataract extraction and intraocular lens; CF, count fingers; Preop BCVA, preoperative best-corrected visual acuity; SMD, senile macular degeneration.
*Each patient in the individual pedigree has a unique identifying patient number. Patient identification numbers for pedigrees A and B are also listed on the individual pedigree for family A.
^{†}Additional ocular surgical procedures, such as CE IOL.

### III. Example 3

In this example, three candidate genes that can be involved in lipid metabolism and/or are expressed in the cornea were analyzed, for the purpose of further understanding SCCD.

DNA samples were obtained from six families with clinically confirmed SCCD. Analysis of FRAP1, ANGPTL7, and UBIAD1 was performed by PCR-based DNA sequencing, to examine protein-coding regions, RNA splice junctions, and 5′ untranslated region (UTR) exons.

No disease-causing mutations were found in the FRAP1 or ANGPTL7 gene. A mutation in UBIAD1 was identified in all six families: Five families had the same N102S mutation, and one family had a G177R mutation. Predictions of the protein structure indicated that a prenyl-transferase domain and several transmembrane helices are affected by these mutations. Each mutation cosegregated with the disease in four families with DNA samples from both affected and unaffected individuals. Mutations were not observed in 100 control DNA samples (200 chromosomes).

Nonsynonymous mutations in the UBIAD1 gene were detected in six SCCD families, and a potential mutation hot spot was observed at amino acid N102. The mutations are expected to interfere with the function of the UBIAD1 protein, since they are located in highly conserved and structurally important domains. (Weiss, Invest Opthalmol Vis Sci. 2007; 48:5007-5012) (DOI:10.1167/iovs.07-0845).

SCCD is considered to be a rare dystrophy, with fewer than 150 articles in the published literature, and most articles reporting only a few affected persons. In the late 1980s, four large Swede-Finn pedigrees of patients with SCCD in central Massachusetts and published the results of clinical examinations of 33 affected individuals. (Weiss, Cornea 1992:11:93-101; Weiss, Opthalmology 1996:103:465-473).

In two of the original Swede-Finn pedigrees, a genome-wide DNA linkage analysis mapped the SCCD locus within a 16-cM interval between markers D1S2633 and D1S228 on chromosome short arm I, region 36.7. In a subsequent study, 13 pedigrees were used to perform haplotype analysis by using densely spaced microsatellite markers refining the candidate interval to 2.32 Mbp between markers D1S1160 and D1S1635. A founder effect was implied by the common disease haplotype that was present in the initial Swede-Finn pedigrees. Identity by state was present in all 13 families for two markers, D1S244 and D1S3153, further narrowing the candidate region to 1.57 Mbp. (Rieheling P, et al., Opthalmologe 2003; 100:979-983; Theendakara, et al., Hum Genet. 2004; 114:594-600.).

Candidate gene analyses for mutations by sequencing the exonic regions of ENO1, CA6, LOC127324, SLC2A5, SLC25A33, PIK3CD, MINI, CTNNBIP1, LZIC, NMNAT, RBP7, UBE4B, K1F1B, PGD, CORT, DFFA, and PEXI4 have been performed. (Aldave, et al., Mol. Vis. 2005; 11:713-716). No pathogenic mutations were found. In May 2007, Oleynikov et al., (IOVS 2007; 48:ARVO E-Abstract 549) reported results of mutation screening of the remaining 16 of the 31 genes that were within the 2.32-Mbp candidate region for SCCD on the short arm of chromosome 1. They found no disease-causing mutations in the patients with SCCD. Possible explanations for the absence of mutations in any of the 31 genes studied included locus heterogeneity for SCCD, incomplete gene annotation for the candidate interval, the presence of pathogenic mutations outside the coding regions of candidate genes, or an error in the assignment of the candidate locus for SCCD due to misclassification of disease status in family members. Indeed, reanalysis of the pedigrees reported in an article by Theendakara et al., (Theendakara, et al., Hum Genet. 2004; 114:594-600) showed a misclassification in one individual. Individual III-5 in Family 9 was reported by herself and her father not to have SCCD. Re-review of the patient's clinical chart, however, revealed that she had evidence of subtle SCCD without crystals. The phenotype in the patient's family was atypical, with some affected members having had only a diffuse, confluent corneal clouding without crystal deposition. (Weiss, et al., Trans Am Opthalmol Soc 2007; 105:616-648).

In a recent article (Weiss, et al., Trans Am Opthalmol Soc 2007; 105:616-648) detailing the phenotypic variations and long-term visual morbidity in 33 pedigrees with SCCD, Family 9 was identified as Family J. When compared with the corneal findings in other SCCD families, the dystrophy phenotype in Family 9 appeared to be mild, resulting in less visual morbidity than in other SCCD pedigrees. Affected members of Family 9 often maintained excellent visual acuity well into old age. Family 9 had been used to define the centromeric boundary of the candidate interval at D151635.9. Family 9 was removed from the analysis and the haplotypes were re-evaluated in only the other 12 families. This resulted in a shift of the centromeric boundary of the candidate interval from D1S1635 to D1S2667. The expanded candidate interval included C1orf127, TARDBP, MASP2, SRM, EXOSC10, FRAP1, ANGPTL7, UBIAD1, and LOC39906. Three genes were chosen: ANGPTL7 (NCBI Entrez Gene ID: 10218; http://www.ncbi.nlm.nih.gov/gene; provided in the public domain by the National Center for Biotechnology Information, Bethesda, Md.), FRAP1 (NCBI Entrez Gene ID: 2475), and UBIAD1 (NCBI Entrez Gene ID: 29914); for initial examination. ANGPTL7 and UBIAD1 were included in the study, because both were expressed in the cornea. FRAP1 and UBIAD1 were included because of their known involvement in lipid metabolism, diabetes, and nutrient signaling. (Parent R, et al., Cancer Res. 2007; 67:4337-4345; McGarvey, et al., Oncogene 2001; 20:1042-1051; McGarvey, et al., Prostate 2003; 54:144-155; McGarvey, et al., J Cell Biochem 2005; 95:419-428; van Gelderen B E, et al., Invest Opthalmol Vis Sci 1998; 39:1782-1788).

### A. Methods

1. Sample Collection

The recruitment efforts which spanned from 1987 to the present have been described in prior publications with institutional Review Board approval of the study obtained from University of Massachusetts Medical Center from 1992 to 1995 and subsequently from Wayne State University to the present. Written informed consent was obtained from all adult participants and the parents of minor participants according to the research tenets of the Declaration of Helsinki Ophthalmic examination included assessment of visual acuity and performance of slit lamp examination to assess corneal findings. Blood samples were collected from individuals from six unrelated SCCD pedigrees. Three of these pedigrees had DNA samples available on at least four individuals (FIGS. 1, 2, 3). Genotyping of two of these families, Q and Y, has been reported. They were identified as pedigrees 11 and 12, respectively, in the article by Theendakara et al. Genotyping of Family T was not reported by Theendakara. DNA from two individuals in Family U, one affected and one unaffected as well as a single affected member from two additional families were also examined. The six families with SCCD were Caucasian, with one family from Germany, two families from England, and three American families, one of mixed European ancestry and the others of unknown ancestry. An independent set of 100 commercially available normal Caucasian DNA samples from individuals of European ancestry (Coriell Cell Repositories, Camden, N.J.) was examined for each mutation, to ensure that mutations were novel, associated with SCCD disease, and were not rare SNPs.

2. DNA Isolation and PCR

DNA Isolation and PCR performed as described in section I, A, 2 above.

3. DNA Sequencing

DNA Sequencing performed as described in section I, A, 3 above.

### B. Results

All protein coding regions, splice junctions, and 5′ untranslated region (UTR) exons were examined in the FRAP1, ANGPTL7, and UBIAD1 genes. Sequence variants were found in the FRAP1 and ANGPTL7 genes, but they were either present in both affected and unaffected individuals or were annotated in the SNP database (dbSNP, data not shown). In UBIAD1, DNA sequencing revealed mutations in affected members of all six families examined (Table 7, FIG. 5). In Family Q (FIG. 1), two affected and two unaffected individuals were sequenced, and both of the affected members (II-10 and III-11) shared the N102S mutation, whereas the unaffected ones (1-1 and 11-9) did not have this mutation. Both affected persons showed evidence of corneal crystal deposition on slit lamp examination. The clinical status of 111-12, a 19-year-old female who had been classified as unaffected in an earlier study (Theendakara, et al., Hum Genet. 2004; 114:594-600) was not clear. The examiner was unsure whether this patient might have a slight corneal haze suggestive of early SCCD without crystals. Sequencing revealed that she had an allele with the N102S mutation in two independent DNA samples, reducing the likelihood of sample mislabeling or other technical errors. It was noted that the disease haplotype was shared by all three affected individuals after haplotype reconstruction, using the corrected clinical classification. (Theendakara, et al., Hum Genet. 2004; 114:594-600).

TABLE 7
Mutations Identified in Six SCCD Families

| Family and | | |
|---|---|---|
| Individual ID | Mutation | Codon |
| T III-3 | GGT > CGT | G177R |
| Q II-11 | AAC < AGC | N102S |
| Y II-3 | AAC < AGC | N102S |
| U | AAC > AGC | N102S |
| BB1 | AAC > AGC | N102S |
| BB2 | AAC > AGC | N102S |

Family T (FIG. 2) was found to have a G177R mutation in both affected siblings (III-2 and 111-3) available for the study and in neither of the two unaffected children (IV-1 and IV-2) of individual III-2. An unaffected spouse (III-4) also did not have the mutation. The third SCCD family, Family Y (FIG. 3), had the same mutation as Family Q in all five affected members available for the study. The one unaffected sibling (III-6) and her unaffected mother (II-4), whose DNA was also sequenced, did not have the mutation.

The N102S mutation was also found in three other unrelated, small SCCD families. An affected individual from Family U possessed the N102S mutation, whereas the unaffected sibling did not. Finally, the N102S mutation was found in two additional families (BB1 and BB2), each one with one affected individual available for the study. The ethnicity of the five unrelated pedigrees with the N102S mutation varied. Family Y was from Germany, families Q and U were from the United States, and families BB1 and BB2 were from England.

In summary, all the 12 definitively affected individuals analyzed in the six families had alterations that were not found in any of the 7 unaffected blood relatives. The only exception was one individual who had a mutation, but whose clinical phenotype was indecisive. Each mutation therefore cosegregated with the disease and was not seen in any of those family members who were definitively diagnosed on slit lamp examination as unaffected. Furthermore, the UBIAD1 gene was examined in 100 Caucasian control DNAs from normal individuals of European ancestry, and neither alteration was observed.

Both mutations changed highly conserved bases and led to substitutions of amino acids conserved in 11 of 12 vertebrate species ranging from telostomes to human. The only species that diverged at N102S was the platypus, which had an isoleucine at amino acid 102, and the armadillo, which had two amino acids deleted at G177R. This evolutionary conservation potentially indicates key roles for these amino acids in normal function of the protein. The UBIAD1 locus produces five transcripts that share exon 1, but exons 2 through 5 are transcript specific. Also, transcripts A, C, D, and F, share exons 1 and 2, which comprise the curated UBIAD1 transcript (RefSeq NM_{—}013319; FIG. 5). The predicted protein structure for transcript A is shown in FIG. 28.

### C. Discussion

1. Difficulty of Making the Diagnosis

Despite the name, Schnyder crystalline corneal dystrophy, only 50% of affected patients have been reported to demonstrate corneal crystals. (Weiss, Cornea 1992:11:93-101; Weiss, Opthalmology 1996:103:465-473; Weiss, et al., Trans Am Opthalmol Soc 2007; 105:616-648). Nevertheless, the pattern of progressive corneal opacification is predictable based on age, regardless of the presence or absence of crystalline deposition. (Weiss, Cornea 1992:11:93-101). Although SCCD with crystals can be diagnosed as early as 17 months of age, diagnosis of SCCD without crystals can be delayed to the fourth decade, because it is difficult to determine when the cornea demonstrates the first changes of subtle panstromal haze. (Weiss, Cornea 1992:11:93-101; Weiss, Opthalmology 1996:103:465-473; Weiss, et al., Trans Am Opthalmol Soc 2007; 105:616-648). Consequently, the assignment of an unaffected phenotype is more challenging in younger patients and can explain the findings in the 19-year-old female patient (111-12 in pedigree Q) who had been classified as clinically unaffected. (Theendakara, et al., Hum Genet. 2004; 114:594-600). This patient possessed the disease haplotype and the mutation (N1025), which was also found in her affected brother, father (FIG. 7), and two paternal aunts. The alternative explanation is incomplete penetrance, a common phenomenon.

2. Corneal Lipid Deposition in SCCD

Corneal arcus has been found to develop in patients with SCCD by 23 years of age (Weiss, Cornea 1992:11:93-101). While premature occurrence of corneal arcus is reported to be associated with coronary artery disease (Halfon et al., Br J Opthalmol 1984; 68:603-604; Rouhiainen, et al., Cornea 1993; 12:142-145; Virchow, Virchows Arch Pathol Anat 1852; 4:261-372), it can occur independent of abnormal lipid levels or other systemic disorders. (Barchiesi, et al., Surer Opthalmol 1991; 36:1-22). Hypercholesterolemia is present in up to two thirds of patients with SCCD. Aldave, et al., Mol Vis 2005:11:713-716; Karseras, et al., Br J Opthalmol 1970; 54:659-662; Williams, et al., Trans Opthalmol Soc UK 1971; 91:531-541) Although familial hypertriglyceridemia and dysbetalipoproteinemia have been reported, familial hypercholesterolemia is the most common lipoprotein abnormality (Crispin, Prog Retin Eye Res 2002; 21:169-224) in patients with SCCD. These abnormalities can also occur in members of the SCCD pedigrees who are reported to be unaffected by the corneal dystrophy. (Barchiesi, et al., Surer Opthalmol 1991; 36:1-22; Bron, et al., Br J Opthalmol 1972:56:383-399; Yamada. et al., Br J Opthalmol 1998; 82:444-447) By comparison, the Cavalier King Charles Spaniel and Rough Collie breeds of dog with crystalline dystrophy usually have normal serum lipid levels. (Crispin, et al., Clin Sci 1988; 74:12).

Previously, the systemic hyperlipidemia in SCCD was postulated to be the primary defect that results in corneal clouding (Bonnet, et al., Bull Soc Ophtalmol Fr 1934; 46:225-229) but this theory lost favor when others documented that patients affected with SCCD can have either normal or abnormal scrum lipid, lipoprotein, or cholesterol levels and that the progress of the corneal opacification is not related to the serum lipid levels. (Lisch, et al., Ophthalmic Paediatr Genet. 1986:7:45-56). Lisch followed 13 patients with SCCD for 9 years and concluded that no link could he drawn between the corneal findings and systemic hyperlipidemia, although 8 of 12 patients had elevated cholesterol or apolipoprotein B levels and 6 of 8 had dyslipoproteinemia type IIa. (Lisch, et al., Ophthalmic Paediatr Genet. 1986:7:45-56).

It has been proposed that the mutated gene responsible for SCCD results in an imbalance in local factors affecting lipid/cholesterol transport or metabolism. A temperature-dependent enzyme defect has been postulated because the initial cholesterol deposition occurs in the axial/paraxial cornea, which is the coolest part of the cornea. (Crispin, Prog Retin Eye Res. 2002; 21:169-224; Burns, et al., Trans Am Opthalmol Soc 1978:76:184-196). Burns et al, documented the cornea as an active uptake and storage site for cholesterol. (Burns, et al., Trans Am Opthalmol Soc 1978:76:184-196). They injected radiolabeled 14C-cholesterol 11 days before removing a patient's cornea during PKP and demonstrated that the level of radiolabeled cholesterol was higher in the cornea than in the serum at the time of surgery. (Burns, et al., Trans Am Opthalmol Soc 1978:76:184-196) Furthermore, lipid analysis of the corneal specimens from patients affected with SCCD who have undergone PKP revealed that the apolipoprotein constituents of HDL (apo A-1, A-II, and E) were accumulated in the central cornea, whereas those of LDL (apo B) were absent. This suggests an abnormality confined to HDL metabolism. (Gaynor, et al., Arterioscler Tbronyb Vasc Biol 1996; 16:992-999).

Because of its smaller size, HDL would be the only lipoprotein that could freely diffuse, while intact, to the central cornea. The size of the larger lipoproteins would prevent their free diffusion unless they were modified (Bron, Cornea 1989; 8:135-140). HDL concentrations are inversely related to the incidence of coronary atherosclerosis (Mayes, et al., Harper's Biochemistry 1993; 23:266-278). Consequently, SCCD lipid accumulation could he caused by a local defect of HDL metabolism. Alternatively, because HDL-related apolipoproteins tend to associate with lipid, the accumulation of these apolipoproteins in the cornea could be secondary to lipid that accumulates in the cornea for some other reason.

The notion that the gene for SCCD plays an important role in lipid-lipoprotein metabolism throughout the body is supported in a report by Battisti et al., (Battisti, et al., Am J Med Genet. 1998; 75:35-39) who cultured the skin fibroblasts of a patient with SCCD. Although membrane-bound spherical vacuoles with lipid materials suggesting storage lipids were present in the skin, there are no other reports in the literature that their experiments have been repeated.

3. UBIAD1 and Lipid Metabolism

UBIAD1 is of interest, as this gene produces a protein that is predicted to contain several transmembrane helices and a prenyltransferase domain that could play a role in cholesterol metabolism. UBIAD1 was previously known as TERE1 (transitional epithelia response protein 1 or RP4-796F18) and the transcript is present in most normal human tissues, including the cornea. (McGarvey, et al., Oncogene 2001; 20:1042-1051). Although there is significant evidence that the RefSeq transcript (2 exons) is in the cornea, evidence of specific expression of the longer transcripts in the cornea is inconclusive. Expressed sequence tags have been isolated from the cornea but information about specific localization of the protein within the cornea is not known. McGarvey et al., (McGarvey, et al., Prostate 2003; 54:144-155) demonstrated that the expression of this gene is greatly decreased in prostate carcinoma. UBIAD1 interacts with the C-terminal portion of apo E (McGarvey, et al., Prostate 2003; 54:144-155; McGarvey, et al., J Cell Biochem 2005; 95:419-428), which is known to be important in reverse cholesterol transport, because it helps mediate cholesterol solubilization and removal from cells. (Knob, et al., J Biol Chem 1994; 269:24511-24518; Zhang, et al., J Biol Chem 1996; 271:28641-28646). Apolipoprotcin E has been found to be present at increased levels in corneal specimens from SCCD corneas. (Gaynor, et al., Arterioscler Tbronyb Vasc Biol 1996; 16:992-999). Consequently, a potential mechanism for UBIAD1-mediated cornea lipid cholesterol accumulation in the cornea is that altered interaction with apo E, and possibly other HDL lipid solubilizing apolipoproteins, results in decreased cholesterol removal from the cornea.

There is another possible mechanism by which a mutation in the UBIAD1 gene could cause corneal cholesterol accumulation. This gene contains a prenyl-transferase domain, suggesting that the gene can function in cholesterol synthesis. Prenylation reactions are involved in cholesterol synthesis and the synthesis of geranylgeraniol, an inhibitor of HMG-CoA reductase, the rate limiting enzyme in cholesterol synthesis. (Sever, et al., J Biol Chem 2003; 278:52479-5 2490). Thus, it is possible that UBIAD1 functions in regulating cholesterol synthesis and that excess cholesterol synthesis occurs when this gene is defective. In this regard, increased cholesterol synthesis in the liver and other tissues would be expected to down-regulate the LDL receptor that mediates removal of LDL from the blood, thus accounting for the elevated LDI blood levels often observed in patients with SCCD.

The potential consequences of the mutations described in this study on UBIAD1 protein function should be investigated. The occurrence of the N102S mutation in five unrelated SCCD families of different ethnicity suggests that this can be a mutation hot spot. The location of these alterations relative to the structure of the protein in the membrane is also interesting. Both occur at sites in the protein where transmembrane helices exit the membrane and thus are located at the hydrophichydrophilic interface. Altered organization of the protein in the membrane can affect prenyl-transterase activity or alter interactions with substrates of binding partners. The UBIAD1 locus produces five transcripts that share exon 1, but exons 2 through 5 are transcript specific. An expanded mutation spectrum can help identify which transcript produces the protein that, when mutated, causes SCCD. Furthermore, an expanded spectrum of mutations can assist in identification of genotypephenotype correlations that highlight specific functions of the protein that, when mutated, lead to family-specific SCCD characteristics. Orr et al., (Orr, et al., PLoS ONE 2007; 2(8):e685) have published independent results with mutations in the UBIAD1 gene in five unrelated families. Of interest, one of the families had the N102S mutation that was present in five of the families.

### IV. EXAMPLE 4

### A. Introduction

Recently, six different mutations on the UBIAD1 gene on chromosome 1p36 were found to result in SCCD. The purpose of this article is to further characterize the mutation spectrum of SCCD and identify structural and functional consequences for UBIAD1 protein activity. DNA sequencing was performed on samples from 36 individuals from 14 SCCD families. One affected individual was an African American and SCCD has not been previously reported in this ethnic group. UBIAD1 mutations were identified in all 14 families which had 30 affected and 6 unaffected individuals. Eight different UBIAD1 mutations, 5 novel (L121F, D118G, and S171P in exon 1, G186R and D236E in exon 2) were identified. In four families with DNA samples from both affected and unaffected individuals, the D118G, G186R, T1751, and G177R mutations cosegregated with SCCD. The genetic mutation in UBIAD1 has been identified in 20 unrelated families with 10 (including 5 reported here), having the N102S mutation. The results suggest that N102S can be a mutation hot spot because the affected families were unrelated including Caucasian and Asian individuals. There was no genotype phenotype correlation except for the T1751 mutation which demonstrated prominent diffuse corneal haze, typically without corneal crystals. Protein analysis revealed structural and functional implications of SCCD mutations which can affect UBIAD1 function, ligand binding and interaction with binding partners, like apo E.

A retrospective review of 115 affected individuals from 34 SCCD families identified by one of the authors (Weiss) since 1989 showed that these families demonstrated corneal opacification that followed the predictable progressive pattern dependent on age. (Weiss, Trans Am Opthalmol Soc 2007; 105:616-648). All patients demonstrated central or paracentral corneal crystals, central or paracentral corneal haze, or a combination of both findings. Approximately 50% of patients had the characteristic superficial corneal crystalline deposits. Although the youngest patient in this series was diagnosed at 17 months of age, the clinical diagnosis has been reported to be delayed up to the fourth decade (Weiss, Opthalmology 1996; 103:465-473) if crystalline deposits are absent. In addition to hypercholesterolemia, the only other systemic finding that has been associated with SCCD is genu valgum, which is also thought to be an independent trait. Of the 115 individuals from 34 families with SCCD, genu valgum was noted in only five individuals from three families (Weiss, Trans Am Opthalmol Soc 2007; 105:616-648).

Although many patients maintained surprisingly good visual acuity until mid age, complaints of glare and loss of daytime visual acuity did increase with age. PKP surgery, to remove the opacified cornea, was reported in 20 of 37 (54%,) patients >50 years of age and 10 of 13 (77%) of patients >70 years of age indicating that the disease is a cause of significant visual morbidity. The only other treatment for visual loss in SCCD is the use of PTK, which is the application of excimer laser to ablate the surface cornea in order to remove the anterior corneal stromal cholesterol crystals. The cornea dystrophy can recur after PKP and PTK but at the present time, there are no other treatments for this disease. Genetic analysis will aid patient identification and can facilitate development of effective treatment.

In 1996, a genome-wide DNA linkage analysis in two SCCD families was used to map the SCCD locus within a 16 cM interval between markers D1S2633 and D1S228 on chromosome 1p36 (Shearman, et al., Hum Mol Genet. 1996; 5:1667-1672). The results of haplotype analysis on 13 pedigrees which refined the candidate interval to 2.32 Mbp between markers D1S1160 and D1S1635 was subsequently reported. Identity by state was present in all 13 families for two markers, D1S244 and D1S3153, further narrowing the candidate region to 1.57 Mbp. (Riebeling, et al., Opthalmologe 2003; 100:979-983; Theendakara, et al., Hum Genet. 2004; 114:594-600). Recently, it was reported that mutations in the UBIAD1 gene resulted in SCCD (Weiss, Trans Am Opthalmol Soc 2007; 105:616-648) in six families with two different mutations, N102S and G177R. On et al., independently described five SCCD families with five distinct mutations: N102S, D112G, R119G, T1751, and N232S. (Orr, et al., PLoS ONE 2007; 2(8):e685).

The UBIAD1 gene spans 22 kb and the locus contains up to five exons with potentially several different transcripts. To date, mutations have only been described in exons 1 and 2 which form a discrete transcript encoding a protein with a predicted prenyl transferase domain and up to eight transmembrane spanning regions. To define the mutation spectrum in SCCD further, DNA sequencing was performed on samples from affected and unaffected individuals originating from 14 apparently unrelated families of varying ethnicities. One of the families was African American. SCCD has not previously been reported in the literature in a family of this ethnicity.

### B. Methods

1. Patient and Sample Collection

The recruitment efforts which spanned from 1987 to the present have been described in prior publications (Shearman, et al., Hum Mol Genet. 1996; 5:1667-1672; Theendakara, et al., Hum Genet. 2004; 114:594-600) with Institutional Review Board approval of the study obtained from University of Massachusetts Medical Center from 1992 to 1995 and subsequently from Wayne State University to the present. Written informed consent was obtained from all adults and the parents of minors under research tenets of the Declaration of Helsinki. Opthalmologic examination included assessment of visual acuity and performance of slit-lamp examination to assess corneal findings. When the information was available, the characteristics and location of the corneal opacity was recorded. Notation was made whether there was a central (or paracentral) opacity, corneal crystals, mid peripheral opacity and/or arcus lipoides on clinical examination. Slit-lamp photographs were obtained when possible for further documentation of corneal findings. Blood samples were collected from family members from 14 apparently unrelated pedigrees (Table 8).

TABLE 8
Mutations in UBIAD1 in New Families With SCCD

| Family | Ethnicity | Gene mutation^{a} | Protein^{b} | Exon | Loop^{c} | Affected^{d} | Unaffected^{e} |
|---|---|---|---|---|---|---|---|
| BB | Czech^{f} | 637 A > G | N102S | 1 | 3 | 1 | 0 |
| DD | Taiwanese | 637 A > G | N102S | 1 | 1 | 1 | 0 |
| K | German | 637 A > G | N102S | 1 | 1 | 5 | 0 |
| L | American | 637 A > G | N102S | 1 | 1 | 1 | 0 |
| R | American | 637 A > G | N102S | 1 | 1 | 1 | 0 |
| BB3 | British | 693 C > T | L121F | 1 | 1 | 2 | 0 |
| O | American | 693 C > T | L121F | 1 | 2 | 2 | 0 |
| H | American | 685 A > G | D118G | 1 | 2 | 1 | 1 |
| G | German-American | 888 G > A | G186R | 2 | 2 | 2 | 5 |
| J | Hungarian-American | 856 C > T | T175I | 1 | 2 | 8 | 1 |
| K1 | German | 843 T > C | S171P | 1 | 2 | 2 | 0 |
| X | Taiwanese | 861 G > A | G177R | 1 | 2 | 1 | 0 |
| Z | Kosovar | 861 G > A | G177R | 1 | 2 | 2 | 1 |
| FF | African-American 1 | 1040 C > G | D236E | 2 | 3 | 1 | 0 |

^{a}Location of mutation in RefSeq NM_013319
^{b}Predicted effect of genetic mutation on protien NP_037451
^{c}Loop see FIG. 3B.
^{d}Affected, number of affected indviduals with DNA sequence information in the family
^{e}Unaffected, number of unaffected indviduals with DNA sequence information in the family
^{f}Czechoslovakian

No genetic studies had been carried out previously on 10 of the 14 families, Families BB, BB3, FF, DD, H, L, O, R, X, and Z; whereas four of the families had been previously used for haplotype studies (Theendakara, et al., Hum Genet. 2004; 114:594-600). Families G and J were called pedigrees 8 and 10, respectively, in the article by Theendakara et al., (Theendakara, et al., Hum Genet. 2004; 114:594-600). Families K and K1 were called pedigrees I and II, respectively, in the article by Lisch et al. (Lisch, et al., Ophthalmic Paediatr Genet. 1986; 7:45-56). Control samples were 100 commercially available normal Caucasian DNA samples (the Coriell Institute for Medical Research) which were examined for each mutation to insure that mutations were novel, associated with SCCD disease, and were not rare sequence variants.

2. DNA Isolation, PCR, and Sequencing

Genomic DNA was isolated from blood using the PUREGENE® DNA isolation kit (Gentra Systems, Minneapolis, Minn.). PCR products were designed to amplify exons and RNA splice junctions. Amplification of DNA and DNA sequencing were described previously [Weiss et al., 2007].

3. Protein Informatics

Analysis of the protein hydrophobicity for membrane spanning regions (transmembrane regions) was achieved using several programs: Sosui (Hirokawa, et al., Bioinformatics 14:378-389), TMPred (Hofmann, et al., Biol Chem Hoppe-Seyler 1993; 374:166) TMHMM ALOM/PSort (Nakai, et al., Trends Biochem Sci 1999; 24:34-36), and MEMSAT 3 [Tones, et al., 1994]. The output from Pongo that incorporated predictions from several of these programs was useful for generating the consensus structure of the protein in the membrane. (Amico, et al., Nucleic Acids Res 2006; 34 (Web Server issue): W169-W172). Consensus transmembrane regions were derived by visually aligning and comparing graphical displays of protein hydrophobicity. TOPO2 was used to display and annotate these results (Johns, TOPO2, Transmembrane_protein_display_software, [www.sacs.ucsf.edu/TOPO2/]). The amino acid sequences of UBIAD1 from multiple species and other related proteins were obtained from the NCBI protein database [http://www.ncbi.nlm.nih.gov]. This included UBIAD1 from human (Q9Y5Z9), mouse (AAH71203), pufferfish (Q4SCA3), chicken (Q5ZKS8), frog (Q28HR4), fruit fly (Q9V3R8), mosquito (AAH71203), human-farnesyltransferase (P49356), para-hydroxybenzoate-polyprenyltransferase/coenzyme Q2 reductase COQ2 (Q96H96), protein prenyltransferase alpha subunit repeat containing 1 [PTAR11 (AAH53622), geranylgeranyltransferase [RABGGTB] (AAH20790), E. coli proteins men A (P32166) and UbiA (POAGK1). The putative polyprenyldiphosphate binding site reported by Suvarna et al., [1998] was used to identify homologous human UBIAD1 amino acids (within the predicted prenyl transferase domain) that were likely binding sites for the UBIAD1 substrate. This was done using MultiAlign. (Corpet, et al., 1998, Nucleic Acids Res 1998; 26:323-326). Clustal W was used to ascertain the divergence of other known prenyl, geranyl, and farnesyl transferases with human UBIAD1 (Chema, et al., Nucleic Acids Res 2003; 31:3497-3500) [http://www.ebi.ac.uk/tools/clustalw/].

4. Phenotype-Genotype Correlation

The clinical data from each individual was reviewed to confirm that the corneal findings were consistent with the diagnosis of SCCD. In order to assess phenotype-genotype associations; there was a review of both the documented corneal findings from clinical examination and the available slit-lamp photographs from affected individuals in families that had undergone mutation analysis. No information about the identity of the individual, family name or mutation was present on the photographs. After the photographs had been categorized, identifying information concerning family and mutation identification was supplied to determine whether the particular corneal findings correlated with specific families or specific mutations.

### C. Results

Altogether 36 DNA samples from 14 SCCD families were examined in sequences corresponding to protein coding regions, splice junctions, and 5′ and 3′ untranslated regions in the UBIAD1 reference sequence (NM_{—}013319, 1,477 bp). The age of the affected individuals ranged from 11 to 80 years of age. DNA sequencing revealed mutations in all 30 affected members and none of the six unaffected members from all 14 families (Table 8). Eight distinct mutations were found including two previously described mutations, N102S (Orr, et al., PLoS ONE 2007; 2(8):e685; Weiss, Trans Am Opthalmol Soc 2007; 105:616-648), G177R [Weiss et al., 2007] and T1751 (Orr, et al., PLoS ONE 2007; 2(8):e685) in exon 1. Novel mutations in exon 1 included L121F (families BB3 and O), D118G (Family H), and S171P (Family K1). Novel mutations in exon 2 were G186R (Family G) and D236E (Family FF). None of the mutations were found in an independent set of 100 commercially available healthy Caucasian DNA samples (200 chromosomes) from individuals of European ancestry.

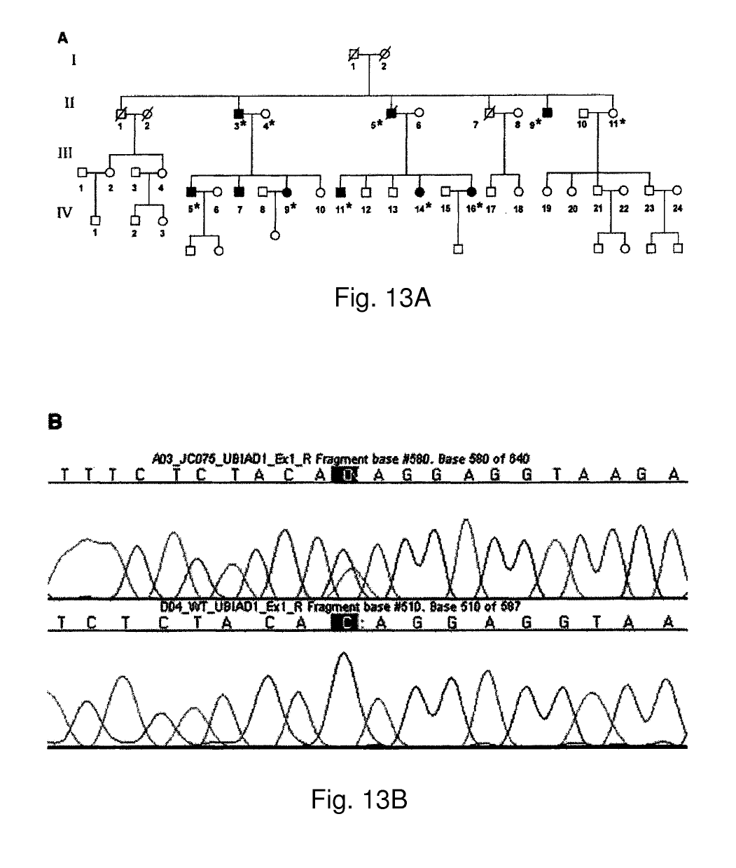

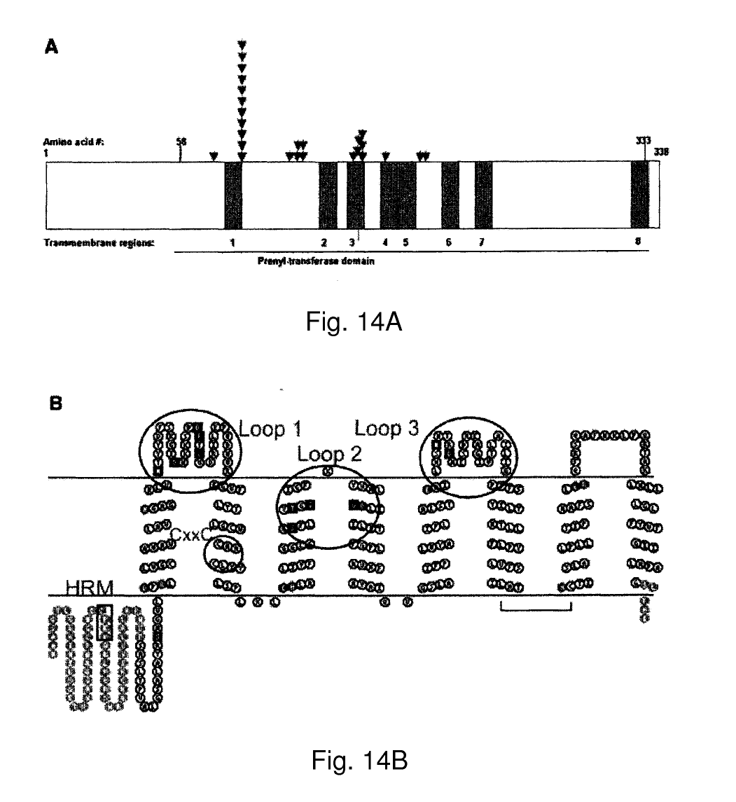

While most of the families were small, consisting of one or two affected individuals, families H, G, J, and Z had both affected and unaffected individuals. New mutations that cosegregated with disease were observed in each of these four families. The D118G alteration in Family H was found in the single affected individual but was not found in her unaffected mother. Although, the father was not available for examination, he was reported as not having SCCD. It is therefore possible, that this could represent a sporadic case. The G1867R mutation in Family G was found in two affected individuals but not the three unaffected individuals or one spouse (FIG. 12A). In Family J, the T1751 mutation was found in eight affected individuals but not one unaffected family member (FIG. 13A) or one spouse. Representative sequence chromatograms demonstrating the identified mutations are shown (FIGS. 12B and 13B). The Family Z mutation G177R was found in the two affected individuals, but not in a single unaffected individual. The only newly described mutation in which cosegregation analysis could not be performed was D236E in Family FF which included only a single affected patient.

1. Ethnicity of Families and Founder Mutations

Both the N102S and the G177R mutation have been described previously (Weiss, et al., Invest Opthalmol Vis Sci 2007; 48:5007-5012).

In the present study, the N102S mutation was found in five families (BB, DD, K, L, and R). Four families were Caucasian with either European (families BB and K) or unknown ethnicity (families L and R). Family DD was Taiwanese. The G177R mutation was found in a Family from Kosovo (Family Z), and another family from Taiwan (Family X).

Eleven of the 14 families were Caucasian with European or unknown ethnicity. This represents a challenge in determining whether these alterations, especially the N102S mutation, are independent or the result of founder mutation. Comparison of additional detailed haplotypes of this locus in these families can help clarify this issue. Three of the 14 families were non-Caucasian. Two Taiwanese families with distinct mutations were described, N102S in family DD and G177R in Family X. In addition, a new SCCD mutation, D236E, was found in the first African American individual reported with the disease (Family FF).

2. Analysis of the Potential

a. Consequences of the Mutations The amino acid substitutions described as mutations in SCCD families were examined for charge, size and hydrophobicity to understand the consequences of these mutations on the UBIAD1 protein structure and function. Many of the mutations reported in this and prior studies (Orr, et al., PLoS ONE 2007; 2(8):e685; Weiss et al., Invest Opthalmol Vis Sci 2007; 48:5007-5012) were nonconservative amino acid substitutions. There were dramatic size and/or shape differences between the reference sequence and mutant amino acids in 7/8 mutations (N102S, D118G, L121F, S171P, T1751, G177R, G186R). Two mutations changed the charge on the amino acid (D118G and G186R). Hydrophilic residues were exchanged with glycines in three mutations (D118G, G177R, and G186R) and hydrophobicity and/or protein structure was altered in S171P and T1751 (hydrophilic to hydrophobic).

The locations of the mutations identified in this as well as two prior publications (Orr, et al., PLoS ONE 2007; 2(8):e685; Weiss, et al., Invest Opthalmol Vis Sci 2007; 48:5007-5012) revealed several clusters of mutations (FIG. 14A). This included the N102S hotspot, the region between transmembrane helices 1 and 2 (D112G, D118G, and R119G-negatively charged reference sequence amino acids altered to neutral glycine) and a cluster of alterations in transmembrane helix 3 (S171P, T1751, G177R). All mutations occurred within the predicted prenyl transferase domain and N102 and G177 occurred at positions where transmembrane helices 1 and 3 (respectively) emerged from the lipid bilayer.

Two-dimensional modeling (FIG. 14B) showed that mutations appear to occur in parts of the protein located on one side of the membrane. It is noted that this observation rests upon the correct number (eight in this model) and location of transmembrane helices. As shown, all alterations fall either in aqueous portions of the UBIAD1 protein or lie in transmembrane helices close to one face of the lipid bilayer (top half of FIG. 14B). The mutations group in three clusters relative to the orientation of the lipid bilayer and UBIAD1 transmembrane helices. These are circled and identified as loops 1, 2, or 3. Each loop contains an aqueous portion of the protein and portions of two transmembrane helices. No alterations were seen in a potential loop 4 (not labeled) or in amino acids on the portion of UBIAD1 facing the other aqueous compartment (on the other side of the lipid bilayer). A putative heme regulatory motif (HRM) at residues 30-34 (X-Xys-Pro-X) is similar to the yeast transcriptional activator (Zhang, et al., EMBO 1995; J14:313-320) and a predicted oxido-reductase motif (Cys-X-X-Cys) at residues 145-148 (Quan, et al., J Biol Chem 2007; 282:28823-28833) do not appear to be affected by SCCD mutations. In silico calculations as to localization of the protein in the cell were inconclusive. Examination of a putative prenyldiphosphate binding site, identified based upon analysis of E. coli UbiA and menA proteins (Suvarna, et al., J Bacteriol 1998; 180:2782-2787) revealed that two alterations, the N102 hotspot and D112 (Orr, et al., PLoS ONE 2007; 2(8):e685), altered highly conserved amino acids (FIG. 14C). The putative active site resides in loop 1 (FIG. 14B). The most commonly mutated residue, N102, appear to be universally conserved among species and is situated precisely where the model predicts transmembrane helix 1 (FIGS. 14A and B) emerges from the membrane. Alignment of the amino acids in the putative ligand or polyprenyldiphosphate binding site from human, mouse, chicken, frog, and pufferfish are identical (FIG. 14C). The putative human ligand binding site shares over 75% homology to fruit fly and mosquito UBIAD1 and 25% homology with residues in E. coli menA and UbiA proteins. Examination of homology (FIG. 14D) places human UBIAD1 as an outlier among other prenyl transferase-like proteins, including human COQ2, PTAR1, farnesyl and geranyl transferases, and E. coli enzymes, UbiA and menA.

b. Genotype-Phenotype Correlation

Except for Family O, every other family had documentation of either slit-lamp examination findings and/or slit-lamp photographs. Family 0 had a diagnosis of SCCD but no record of the details of the corneal exam and no photographs. Detailed clinical exams were available for affected individuals from 12 of the 14 families (BB, BB3, FF, G, H, J, K, K1, L, R, X, and ZZ) and were not available for two families (DD and O). Slit-lamp photographs of the cornea were examined from 21 affected patients from 10 (BB, DD, FF, G, H, J, K, K1, X, and Z) of the 14 families. No photographs were available from families BB3, L, O, or R. There were slit-lamp photographs available for at least one affected patient with each mutation described in this publication.

The clinical findings of all the described families have been previously published. (Weiss, Trans Am Opthalmol Soc 2007; 105:616-648). Systemic cholesterol measurements of affected individuals were not uniformly obtained. Genu valgum was reported in individuals from only families G and Z. Otherwise there were no other physical abnormalities associated with SCCD.

The corneal findings in all families appeared to follow the predictable pattern of progressive corneal opacification previously described in this disease (FIG. 8) (Weiss, Cornea 1992; 11:93-101). Younger individuals demonstrated only central corneal opacification with or without crystalline deposits. Arcus lipoides was noted during approximately the third decade. Finally, the mid-peripheral cornea was noted to become opacified by the end of the fourth decade in most individuals.

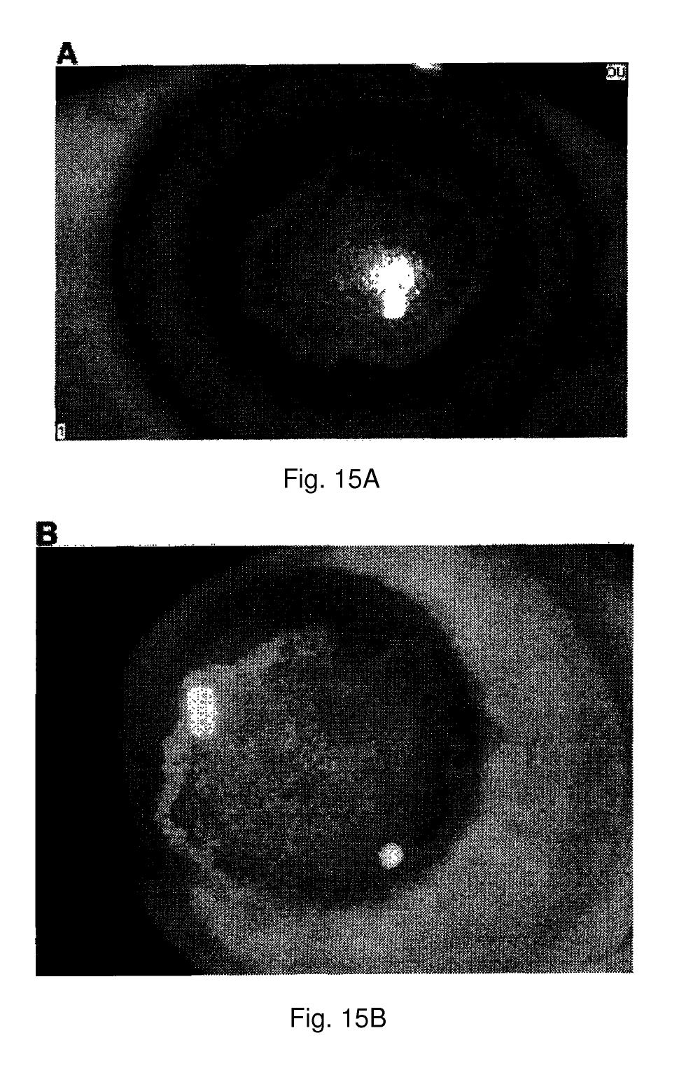

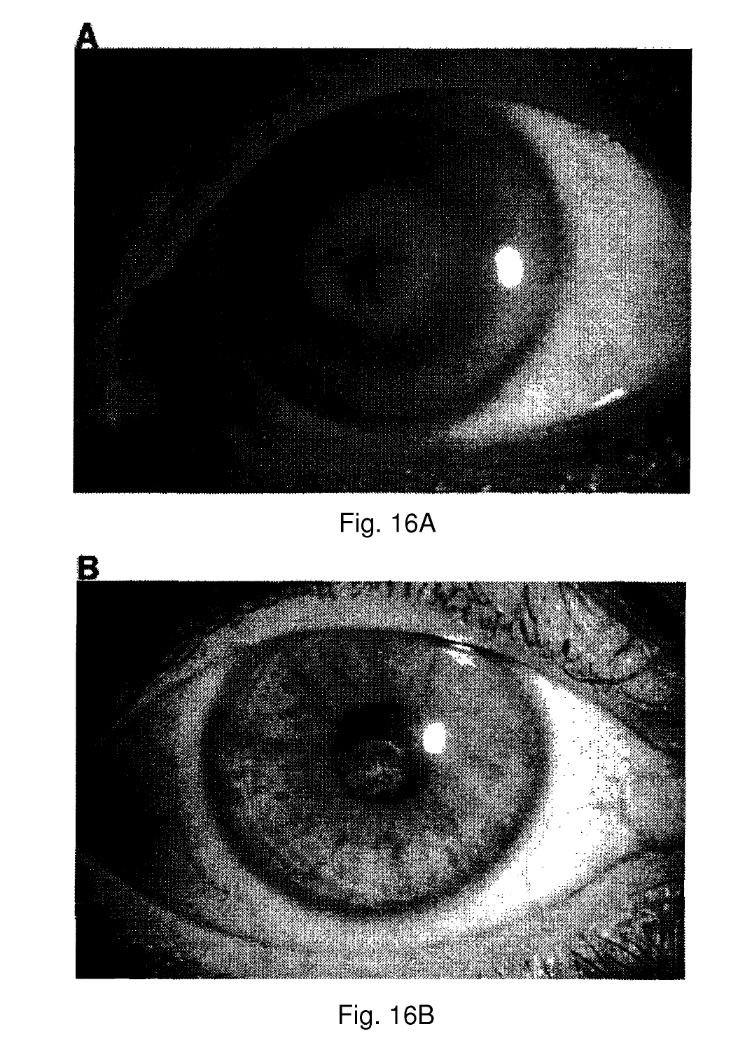

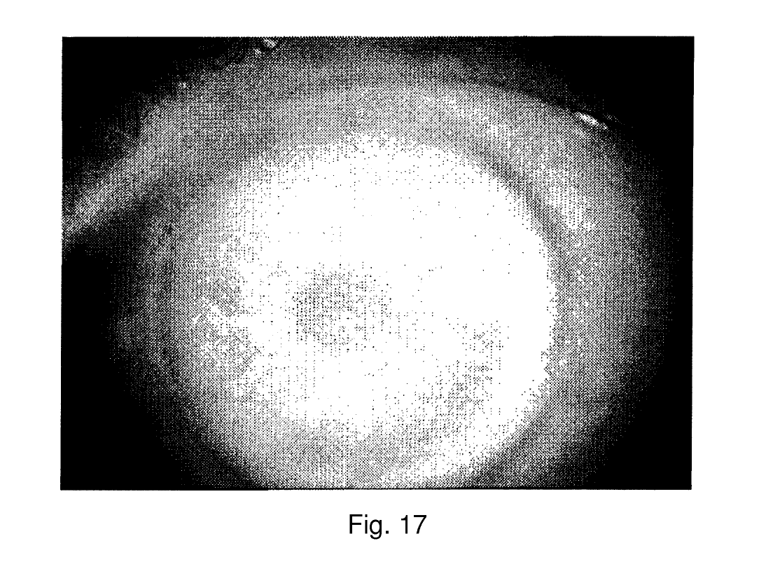

Examination of unlabeled slit-lamp photographs by one of the authors (Weiss) demonstrated that while the approximate age of the patient could be predicted by the corneal opacification pattern; there did not appear any pattern of corneal opacification that was associated with a specific mutation. A 42-year-old African American woman from Family FF (FIG. 15A) with a D236E mutation and a 70-year-old German man from Family K1 (FIG. 15B) with a S171P mutation both demonstrated a denser scalloped ring of crystals surrounding the central corneal opacity. Although families X and Z both had a G177R mutation, the 38-year-old Taiwanese female from Family X (FIG. 16A) had predominantly corneal haze and the 39-year-old male of Kosovo ethnicity from Family Z (FIG. 16B) had predominantly central crystalline deposition. Ring pattern of corneal crystalline deposition was noted in individuals of different ages and with different mutations. This ring pattern of crystals was found in a 26-year-old woman from Family H with the D118G mutation, a 28-year-old woman from Family G with the G186R mutation, a 20-year-old man from Family BB and a 48-year-old woman from Family K; both with the N102S mutation.

Unlike the more typical appearance of SCCD in which three distinct zones of corneal opacification could be detected (central or paracentral, midperipheral, and peripheral) some individuals in Family J had a diffuse confluent corneal opacification (FIG. 17). The most prominent finding in affected individuals in Family J was corneal haze. Only three of eight affected members were noted to have corneal crystalline deposits which did not prominently affect the visual axis.

### D. Discussion

Examination of 30 affected individuals from 14 SCCD families, confirmed the presence of UBIAD1 mutations in all of them. Despite the rarity of this corneal disease, a total of 20 apparently unrelated families that possess mutations in the UBIAD1 gene have been studied, providing strong evidence to support the hypothesis that SCCD is caused by UBIAD1 mutations. The present study of 14 families reports eight distinct mutations, three of which have been described previously, N102S and G177R and T1751 in exon 1. Five mutations are novel, D118G (Family H), L121F (families BB3 and O), and S171P (Family K1) in exon 1 and G186R (Family G) and D236E (Family FF) in exon 2. Analysis of four families included DNA samples from both affected and unaffected individuals. In these families, the respective mutations: D118G, G186R, T1751, and G177R cosegregated with the disease providing further confirmation that these mutations caused SCCD. Including results from Weiss et al., (Weiss, et al., Trans Am Opthalmol Soc 2007; 105:616-648) who described two mutations (N1025 and G177R) and On et al., (Orr, et al., PLoS ONE 2007; 2(8):e685) in which five distinct mutations, N102S, D112G, R119G, T1751, and N232S were described; a total of 11 mutations have been described in the UBIAD1 gene.

Although the majority of articles describe patients with SCCD of European descent, the corneal dystrophy has also been reported in the Asian population; (Orr, et al., PLoS ONE 2007; 2(8):e685; Yamada, et al., Br J Opthalmol 1998; 82:444-447). Two of the 14 pedigrees described in this study were of Asian descent. The mutations detected in these families, N102S (Family DD) and G177R (Family X), were also found in patients of European ethnicity. Unlike Caucasian and Asian populations, SCCD had never previously been reported in an African American individual. Consequently, it is of interest that the African American affected individual from Family FF had a D236E mutation that has not been previously described in other families.

In prior publications, six of 11 SCCD families, presented the mutation, N102S (Orr, et al., PLoS ONE 2007; 2(8):e685;Weiss, et al., Trans Am Opthalmol Soc 2007; 105:616-6480 which led the authors to postulate that this might represent a mutation hot spot. With the addition of five more families from the present study which also demonstrated the N102S mutation; there are a total of 11 (44%) of the reported 25 SCCD families with this mutation. These 11 families are apparently unrelated with varying ethnicities described as two British, two German, one Czechoslovakian, one Taiwanese, and four American with unknown ethnicity. Family F123 from Orr et al., (Orr, et al., PLoS ONE 2007; 2(8):e685) was presumed Italian as they were referred from Italy (Battisti, et al., Am J Med Genet. 1998; 75:35-39). The variation of the ethnic background argues against the likelihood of a founder effect and adds support to the proposal that N102S has been independently mutated in these families and thus can represent a mutational hotspot for SCCD.

While the earliest diagnosis of SCCD has been made at 17 months of age, diagnosis can be delayed to the fourth decade when crystalline deposits are absent. The pattern of corneal opacification in this disease is fairly predictable and depends on age (FIG. 8). The central or paracentral opacity, crystalline or acrystalline is always the first finding which can occur in patients less than 23 years of age. Additionally, the next finding to be noted is arcus lipoides, a peripheral ring opacity which occurs in patients of 23 years of age and older and ultimately patients older than 37 years of age display opacification of the mid-peripheral cornea (Weiss, Cornea 1992; 11:93-101). Delleman and Winkelman (Delleman, et al., Opthalmologica 155:409-426) described different patterns of corneal opacification in SCCD including a ring like central deposit. The corneal findings of the SCCD families described in the current study have been previously published (Weiss, Trans Am Opthalmol Soc 2007; 105:616-648).

The written description of the corneal findings in those individuals, who had mutation analysis, was not sufficiently detailed to distinguish unique phenotypic changes in affected individuals with different mutations. Nevertheless, slit-lamp photographs allowed a visual comparison to determine if there were any morphological differences.

No genotype-phenotype correlation could be made for the majority of mutations. There was phenotypic variation within families. Affected individuals from different families were found who had different mutations but whose clinical findings were virtually identical. Conversely, there were affected individuals from different families that had identical mutations but very different clinical appearance. It is possible that the phenotypic heterogeneity resulted from modulating influences such as environmental effects or that a specific phenotype can be a result of the interaction of multiple genes.

A unique phenotype was noted in Family J. While all affected individuals appeared to have the corneal opacification divided into three corneal zones; individuals in Family J demonstrated a diffuse confluent opacity which was not noted in any other families. This family (FIG. 13) has been previously described to have an unusual phenotype (Weiss, Trans Am Opthalmol Soc 2007; 105:616-648). Despite consultation with numerous corneal subspecialists for more than one decade, individuals in this family had been unsuccessful in obtaining a correct diagnosis for their corneal disease. (Weiss, Trans Am Opthalmol Soc 2007; 105:616-648). In addition, Family J did have a distinct mutation T1751 which was not found in any of the other families that were examined and so it is possible that this mutation is associated with a slightly different clinical presentation of the disease.

The location of amino acid alterations is interesting and can impact the structure of the protein in the membrane (FIG. 14B). Of potential structural significance, the model places the N102S mutation at the position where the first transmembrane helix emerges from the lipid bilayer. Furthermore, all SCCD mutations in the UBIAD1 protein appear to affect only one side of the protein in the membrane (top half of the protein, FIG. 14B) and residues in both aqueous and hydrophobic (transmembrane) portions of the protein are altered in different families. Additional experiments will help clarify the location of the wild-type and mutant protein in a specific membrane and can help clarify why the mutations cluster on one side of the membrane. Future studies are underway that will examine whether mutations affect protein folding and will examine the possibility that mutant UBIAD1 can be retained in the ER, and/or targeted for degradation. Studies examining protein localization will determine if mutant protein is located in the same membrane as wild-type and whether the clinical effects observed in SCCD patients are due to haploinsufficiency. Of specific interest will be examination of the published interaction between UBIAD1 and apolipoprotein E and whether SCCD mutations alter this interaction (McGarvey, et al., J Cell Biochem 2005; 95:419-428).

The polyprenyldiphosphate binding sites in E. coli menA (Suvarna, et al., J Bacteriol 1998; 180:2782-2787) and UbiA (Melzer, et al., Biochem Biophys Acta 1994; 1212:93-102) were used to identify a putative ligand binding site in loop 1 (FIG. 14C) of UBIAD1 from different species, including human. There was 100% sequence homology in the polyprenyldiphosphate binding site between the human, mouse, pufferfish, chicken, and frog. Two of the SCCD mutations, N102S and D112G, are located within this putative binding site. The high degree of conservation across species at this site suggests that SCCD disease can be due to abnormal ligand binding. The locations of additional mutation clusters in loops 2 and 3 can indicate these portions of the protein form a tertiary structure that can contribute towards proper function of the putative active site. As yet, it is not known whether SCCD mutations activate or inhibit UBIAD1 function and the actual ligand that binds UBIAD1 has yet to be experimentally identified.

Comparison of UBIAD1 with other related proteins (FIG. 14C, D) allows speculation about its function. Prenyltransferases are involved in the mevalonate pathway that functions in protein prenylation and vitamin K, ubiquinone, heme A, dolichol, and cholesterol synthesis. The E. coli menA gene encodes a prenyltransferase involved in the vitamin K biosynthetic pathway. (Suvarna, et al., J Bacteriol 1998; 180:2782-2787). Since humans cannot synthesize vitamin K2 and must obtain it from the diet or from bacteria present in the gut, a different function for UBIAD1 is likely. UbiA in E. coli catalyzes the prenylation reaction of the aromatic intermediate p-hydroxybenzoate which is a critical step in the transfer of a prenyl side chain to the benzoquinone frame in ubiquinone biosynthesis. In humans, this step is catalyzed by COQ2 enzyme. (Lopez-Martin, et al., Hum Mol Genet. 2007; 16:1091-1097). Very low overall sequence homology between UBIAD1 and COQ2 suggests a different role for UBIAD1. Conversely, UBIAD1 mRNA expression levels estimated from counts of expressed sequence tags from eye and other tissues appear to be inversely related to COQ2 expression (e.g., UBIAD1 is expressed 11.5-fold higher than COQ2 in eye) perhaps indicating complementary roles for the protein products [www.ncbi.nlm.nihgov/unigene/estprofile]. A recent report of a rnissense mutation in human COQ2 leading to defects of hioenergetics and de novo pyrimidine synthesis is intriguing (Lopez-Martin, et al., Hum Mol Genet. 2007; 16:1091-1097) because the polyprenyl transferase activity in COQ2 mutant fibroblasts is 33-45% that of controls. Interestingly, UBIAD1 mRNA expression levels in human fibroblasts are 3.4-fold higher than COQ2. The presence of residual prenyltransferase activity in human fibroblasts, the increased expression of UBIAD1 relative to COQ2 in some tissues, and plausible functional redundancy of UBIAD1 catalyzing the same reaction as COQ2 suggest the possibility that UBIAD1 can compensate for COQ2 in the ubiquinone pathway in some tissues. A disturbance in ubiquinone, dolichol, or heme A synthesis could have secondary effects on cholesterol metabolism because all the synthetic pathways share a common branch point precursor. Future studies will examine whether expression of mutant and normal UBIAD1 occurs in a tissue-specific manner. The degree of uniqueness of UBIAD1 compared to the proteins examined in FIG. 14 makes it difficult to predict additional functions based on protein homology.

An alternative role for human UBIAD1 is that it can be involved in prenylation of proteins (Naidu, et al., Brain Res 2002; 958:100-111). McGarvey et al., (McGarvey, et al., J Cell Biochem 2005; 95:419-428) have demonstrated that UBIAD1 (also known as TERE1) interacts with the carboxyl terminus of apoE. Secretion of apolipoprotein E (apoE) by brain glia has been suggested to require protein prenylation (Naidu, et al., Brain Res 2002; 958:100-111). It is speculated that UBIAD1 can be involved in prenylation of apoE that is required for trafficking and function of newly synthesized apoE protein. The farnesyltransferase and geranylgeranyltransferase from the mevalonate pathway are involved in prenylation of proteins (Taylor, et al., EMBO J. 2003; 22:5963-5974; Reid, et al., J Mol Biol 2004; 343:417-433). Amino acid sequence alignment, however, reveals minimal homology between UBIAD1, farnesyltransferase or geranylgeranyltransferase thus suggesting that, if UBIAD1 is a protein prenyltransferase, UBIAD1 belongs to a different category of protein prenyltransferase (FIG. 14D). In any case, considering the reported interaction of UBIAD1 and apoE, changes in the protein structure of UBIAD1 could affect apoE-mediated cholesterol solubilization and removal from cells (Zhang, et al., EMBO J. 1995; 14:313-320) and result in accumulation of cholesterol, a typical phenotype seen in the corneas of SCCD patients.

Histopathologic examination of SCCD corneal specimens demonstrates abnormal lipid deposition throughout the corneal stroma with the crystalline deposits which occur in some patients having been shown to be cholesterol (Bonnet, et al., Bull Soc Ophtalmol Fr 1934; 46:225-229; Thiel, et al., Klin Monatsbl Augenheilkd 1977; 171:678-684; Freddo, et al., Cornea 8:170-177; Vesaluoma, et al., Opthalmology 1999; 106:944-951). Lipid analysis demonstrates excess accumulation of unesterified cholesterol, esterified cholesterol, and phospholipid (Weiss, Cornea 1992; 11:93-101). Animal models for SCCD exist with similar histopathology to that found in humans (Virchow, Virchow's Arch Path Anat 1852; 4:261-372; Crispin, et al., J Small Anim Pract 1983; 24:63-83; Crispin, Cornea 1988; 7:149-161; Crispin, Prog Retin Eye Res 2002; 21:169-224). Crystalline stromal dystrophy is the most common canine corneal lipid deposition and is relatively common in the Cavalier King Spaniel, among other breeds. Corneal opacities similar to SCCD have also been produced by feeding a cholestanol-enriched died to BALB/c mice but these are associated with corneal vascularization which is not present in SCCD. In this animal model, the serum cholestanol was 30-40 times normal and the corneal deposits were composed of calcium phosphate and probably cholestanol (Kim, et al., Biochem Biophys Acta 1991; 1085:343-349). If mutations in the UBIAD1 gene are also found in these animal models they can become important for developing future interventional treatment to prevent the visual loss resulting from the progressive corneal opacification which occurs in SCCD. Lastly, mouse models can be useful to examine whether complete knock out of the gene produces even more dramatic symptoms of disease such profound corneal opacification or systemic abnormalities of cholesterol metabolism. Alternatively, if SCCD mutations increase the activity of UBIAD1, over expression of the wild type and mutant protein in mouse and cell lines can yield additional clues (binding partners) about the role of UBIAD1 in lipid and cholesterol metabolism.

Subsequently, a report appeared on-line (Yellore et al., 2007) that described genetic analysis of three additional families with SCCD including one African American family.

UBIAD1 alterations found included N102S and L121F. This report further supports the contention that N102S is a mutation hotspot in UBIAD1 for SCCD.

### V. Example 5

### Schnyder Corneal Dystrophy Mutations Alter Mitochondrial Ubiad1 Activity To Disrupt Cholesterol Metabolism In A Novel Manner

Schnyder corneal dystrophy ([SCD, MIM 121800] (Van Went, et al., Niederl Tijdschr Geneesks 1924; 68:2996-2997; Schnyder, Schweiz Med Wschr 1929; 10:559-571) is an autosomal dominant eye disease characterized by abnormal deposition of cholesterol and phospholipids in the cornea (Rodrigues, et al., Am J Opthalmol 1987; 104: 157-163). The resultant corneal opacification can be progressive and bilateral. Crystals can present in a variety of patterns that are somewhat dependent on age. Of great interest, two-thirds of affected individuals are hypercholesterolemic. (Bron, Cornea 1989; 8: 75-79). Many unaffected individuals in SCD pedigrees also demonstrate hypercholesterolemia, thus it has been postulated that the corneal disease results from a local metabolic defect of cholesterol processing or transport in the cornea.

A review of 115 affected individuals from 34 SCD families identified by since 1989, confirmed the finding that families presented corneal opacification in a predictable progressive pattern dependent on age (Weiss, Trans Am Opthalmol Soc 2007; 105:616-648; Weiss, Cornea 1992; 11: 93-101). All patients demonstrated corneal crystals or haze, or a combination of both findings. While patients have been diagnosed as young as 17 months of age, the diagnosis can be more challenging if crystalline deposits are absent and onset of symptoms can be delayed into the fourth decade. Although many patients maintained surprisingly good visual acuity until middle age, complaints of glare and loss of visual acuity increased with age. Disproportionate loss of photopic vision as compared to scotopic vision was postulated to be caused by light scattering by the corneal lipid deposition. Surgical removal of the opacified cornea was reported in 20 of 37 (54%) patients 50 years of age and 10 of 13 (77%) of patients 70 years of age. (Weiss, Trans Am Opthalmol Soc 2007; 105:616-648).

Recently, several groups described identification of mutations in human SCD patients in a gene with no prior connection to corneal dystrophy or cholesterol metabolism. (Orr, et al., (2007) PLoS ONE 2: e685; Weiss, et al., Invest Opthalmol Vis Sci 2007; 48:5007-5012; Yellore V S, et al., (2007) Mol Vis 13: 1777-1782; Weiss, et al., Am J Med Genet. 2008; 146A:271-283; Kobayashi A, et al., (2009) Opthalmology 116: 1029-1037). The gene, UBIAD1, is predicted to encode a membrane protein that contains a prenyl-transferase domain similar to a bacterial (E. coli) protein, UbiA. The human gene, UBIAD1 spans 22 kb and the locus gives rise to approximately three different transcripts with up to five unique exons. (Weiss, et al., Invest Opthalmol Vis Sci 2007; 48:5007-5012). To date, mutations have been described exclusively in exons 1 and 2, which encode a discrete RefSeq transcript, NM_{—}013319.

Thirty-one apparently unrelated families have been examined and fifteen different mutations have been characterized. Genetic analysis of families revealed a putative mutation hotspot that altered an asparagine at position 102 to a serine reside. (Weiss, et al., Am J Med Genet. 2008; 146A:271-283). Cumulatively, 12/31 (39%) of apparently unrelated families possess this single hotspot alteration. Mutations are uniformly heterozygous, single base DNA changes that generally result in non-conservative substitutions of apparently critical amino acids. The majority of mutations result in amino acid alterations which replace hydrophobic with hydrophilic residues or the converse (i.e., charged residues replaced with neutral or hydrophobic amino acids).

A putative 23 amino acid binding site was identified based upon analysis of Escherichia coli UbiA and menA proteins. (Weiss, et al., Am J Med Genet. 2008; 146A:271-283; Suvarna, et al., J Bacteriol 1998; 180:2782-2787). Alignment of 23 amino acids in the putative ligand (polyprenyl-diphosphate) binding site shows 100% homology between mammals, amphibians, and the pufferfish. Analysis of the human protein revealed that the hotspot amino acid was predicted to reside within the putative active site (diphosphate binding site area) of the prenyltransferase domain and was completely conserved across species. (Weiss, et al., Am J Med Genet. 2008; 146A:271-283). Two SCD mutations alter highly conserved amino acids within the active site, the N102S hotspot and D112G.

Preliminary two-dimensional modeling of protein hydrophobicity revealed that the N₁₀₂ amino acid is situated precisely at a juxtamembrane position where an N-terminus transmembrane helix was predicted to emerge from the membrane. Lastly, examination of homology between related human proteins places UBIAD1 as an outlier among other prenyltransferase-like proteins, including coenzyme Q2 reductase (COQ2), prenyltransferase alpha subunit repeat containing 1 (PTAR1), farnesyl and geranyl transferases, and E. coli enzymes, UbiA and menA.

Two-dimensional modeling of the human protein predicted that UBIAD1 contains up to eight transmembrane spanning regions. In the consensus model (derived using multiple programs (Weiss, et al., Am J Med Genet. 2008; 146A:271-283)), SCD mutations appear to cluster in three regions of the protein all located on one side of the membrane. (Loops 1-3) (Weiss, et al., Am J Med Genet. 2008; 146A:271-283)). Though this observation rests upon the correct number (eight in the model) and location of transmembrane helices, it can be useful as a predictive model to identify residues critical for protein function that can be impaired in SCD patients. These functions might include interaction with a binding partner or prenyltransferase substrate.

The current example presents analysis of additional SCD families. Homology of the UBIAD1 protein across species was examined, including the degree of conservation of amino acid residues mutated in SCD. Subcellular localization of wild type and mutant UBIAD1 was examined in keratocyte cell lines to determine if SCD mutations altered the localization of protein. In order to identify how mutation of UBIAD1 might result in disregulation of cholesterol metabolism, relative amounts of key cholesterol metabolites were examined in N102S mutant UBIAD1SCD patient and control B-cell lines.

Lastly, in order to examine how protein structure-function can be altered due to SCD mutation, human UBIAD1 was examined using protein threading to generate a three-dimensional molecular model. In silico mutations matching SCD familial alterations were introduced and these findings allow discussion of functional implications of disease-alterations, putative substrates, and therapeutic options.

### A. Material and Methods

1. Patient Identification and Sample Collection

Family history, opthalmologic examination, blood samples were obtained on all affected patients. When possible, other family members were also recruited in order to confirm inherited nature of the SCD mutations. Opthalmologic examination included assessment of visual acuity and slit lamp examination of the cornea detailing location and characteristics of the corneal opacity. Notation was made as to presence of central corneal opacity, mid peripheral haze, arcus lipoides and/or corneal crystalline deposition. Slit lamp photographs were obtained whenever possible to aid diagnosis.

2. DNA Extraction and Mutation Analysis

DNA was extracted using standard methods and either PURGENE® (Gentra/Qiagen, Valencia, Calif.) or other Qiagen reagents (All Prep DNA/RNA Kit). Genetic analysis of patient DNA was performed as previously described, except that FastStart PCR reagents (Roche, South San Francisco, Calif.) and ABI (Foster City, Calif.) thermal cyclers were used. Sanger sequencing was performed using Big Dye reagents (ABI) and subjected to chromatography using a 3730 Genetic Analyzer (ABI). Sequence chromatograms were analyzed using Sequencher, v4.8 (GeneCodes, Ann Arbor, Mich.). Over 100 control DNAs from healthy donors were examined by double stranded sequencing for each mutation to insure that mutations were novel, associated with SCD, and unlikely to be rare polymorphisms. Healthy DNA samples were obtained from the Dean Lab database (MD) and the Coriell Institute for Medical Research (Camden, N.J.).

3. Sequence Alignment, Homology, and Phylogeny

The following UBIAD1 sequences from 19 indicated species were identified using the Ensembl database: NP_{—}037451.1 [Homo sapiens, human], XP_{—}001137312.1 Predicted [Pan troglodytes, chimp], XP_{—}544571.1 Predicted [Canis familiaris, canine], XP_{—}585287.3 Predicted [Bos taurus, cattle], XP_{—}001492378.1 Predicted [Equus caballus, horse], NP_{—}082149.1 [Mus musculus, mouse], XP_{—}233672.1 Predicted [Rattus norvegicus, rat], NP_{—}001026050.1 [Gallus gallus, chicken], XP_{—}686705.2 Predicted [Danio rerio, zebra fish], ENSORLT00000000192 Predicted [Oryzias latipes, medaka/killifish], NP_{—}523581.1 [Drosophila melanogaster, fruit fly], XP_{—}001639930.1 Predicted [Nematostella vectensis, sea anemone], XP_{—}001175897.1 Predicted [Strongylocentrotus purpuratus, sea urchin], ENSCP0G00000011678 Predicted [Cavia porcellus, Guinea pig], ENSFCAG00000000057 Predicted [Fells catus, cat], ENSLAFG00000015673 Predicted [Loxodonta Africana, African elephant], ENSPCAG00000003043 Predicted [Procavia capensis, hyrax], ENSMODG00000011080 Predicted [Monodelphis domestica, opposum], ENSTTRG00000001324 Predicted [Tursiops truncates, bottlenose dolphin], ENSPVAG00000014788 Predicted [Pteropus vampyrus, megabat/flying fox]. Alignments were performed using Clustal 2.0.11 (Larkin, et al., Bioinformatics 2007; 23:2947-2948). A global alignment performed on all proteins was followed by local optimization of overlapping, sequential regions of protein in approximately fifty amino acid increments.

4. Localization of Human UBIAD1

Normal human keratocytes were purchased from ScienCell Research Lab (Carlsbad, Calif.). Schnyder disease and normal patient keratocytes were cultured at 37° C. in Fibroblast Medium (catalogue no. 2301) also obtained from ScienCell Research Lab. For immunofluorescence labeling experiments, the keratocytes were rinsed three times with DPBS before fixing with 2% formaldehyde for 10 minutes at room temperature. Cells were then blocked with 10% FBS in DPBS (FBS blocking solution) for 30 minutes, and then treated 15 minutes with avidin/biotin blocker (Vector Laboratories, Burlingame, Calif.) with a DPBS rinse between each step of the procedure described by the manufacturer. Chicken anti-UBIAD1 was diluted to 5 μg/ml in FBS blocking solution containing 0.2% Triton X-100, and incubated with keratocytes for one hour at room temperature. After three five-minute rinses with DPBS, biotinylated goat anti-chicken IgY (catalogue no. 103-065-155 from Jackson Immunoresearch, West Grove, Pa.) diluted to 5 μg/ml in FBS blocking solution was incubated with keratocytes for one hour. This primary labeling of UBIAD1 was then visualized by incubating keratocytes with 5 μg/ml Alexa 594 (red) streptavidin diluted in DPBS (catalog no. S32356, Molecular Probes, Eugene, Oreg.).

To determine the subcellular localization of UBIAD1, keratocytes were further incubated one hour with either 5 μg/ml mouse IgG2b monoclonal anti-protein disulfide isomerase (catalogue no. S34253, Molecular Probes), an endoplasmic reticulum marker; or 5 μg/ml mouse IgG1 monoclonal anti-OXPHOS Complex I, subunit NADH dehydrogenase (catalogue no. A31857, Molecular Probes), a mitochondrial marker. This was followed by incubation with 5 μg/ml Alexa fluor 488 (green) anti-mouse IgG (catalogue no. A11029, Molecular Probes) for one hour to label the subcellular markers. All antibodies were diluted in FBS blocking solution.

5. Cholesterol Measurements

Lymphocytes were isolated from patient blood samples using lymphocyte separation medium and were immortalized using Epstein-Barr virus. Standard culture conditions utilized RPMI 1640 media (Invitrogen), 15% fetal bovine serum (Hyclone, Waltham, Mass.), and 2× L-glutamine (Invitrogen). Six well plates were used to grow approximately 1 million cells per well, which were rinsed three times each with Dulbecco's phosphate-buffered saline (DPBS) plus Mg²⁺, Ca²⁺, and 0.2% bovine serum albumin (BSA), and then DPBS plus Mg²⁺ and Ca²⁺. Cells were harvested from wells by scraping into 1 ml of distilled water, and then processed as described previously (Kruth, et al., J Cell Biol 1995; 129:133-145). Lipids were extracted from an aliquot of cell suspension using the Folch method (Folch, et al., J Biol Chem 1957; 226:497-509). The cholesterol content of cells was determined according to the fluorometric method of Gamble et al., (Gamble, et al., J Lipid Res 1978; 19:1068-1070). Protein content was determined on another aliquot of cell suspension by the method of Lowry et al., using BSA as a standard. (Lowry, et al., J Biol Chem 1951; 193:265-275).

6. Protein Models

UBIAD1 transmembrane helices and topology were analyzed using the HMMTOP program and server (Tusnady, et al., Bioinformatics 2001; 17:849-850; Tusnady, et al., J Mol Biol 1998; 283:489-506). The Brookhaven Protein Data Bank (PDB) and PHYRE (Protein Homology/analogY Recognition Engine) were searched for proteins homologous to UBIAD1 with at least 30% identity in the amino acid sequence and with a resolved X-ray structure using BLASTp. (Tusnády, et al., J Mol Biol 1998; 283:489-506). PHYRE examined sequence alignments and determined the fold family by including secondary structure predictions and alignment of secondary structure elements.

Homology between UBIAD1 and other prenyltransferase domain containing proteins was examined using MOE (Molecular Operating Environment, Chemical Computing Group Inc., Montreal, Canada). Transmembrane helices were manually examined by using available X-ray structures of prenyl converting enzymes as templates, such as prenyl synthases (cyclases), protein prenyl transferases, and the recently developed model of the all-alpha-helical E. coli UbiA prenyltransferase. (Bräuer, et al., Chembiochem 2008; 9:982-992; Bräuer, et al., J Mol Model 2004; 10:317-327). Alignment was done by applying the BLOSUM62 alignment matrix, a gap start penalty of 10, and gap extension of 3 to produce a well fitting superposition of the required alpha helical structural alignment. The positional placement of geranylpyrophosphate and a single magnesium cation were extracted from the E. coli UbiA model and fitted into the model of human UBIAD1. The model obtained from MOE was refined using the molecular dynamics refinement tool YASARA. Stereochemical quality of the model was analyzed with PROCHECK (Laskowski, et al., J Biomol NMR 1996; 8:477-486). All parameters evaluated by PROCHECK are inside or even better (overall G-factor) then required for an analogous X-ray of better than 2 Å resolution. Inspection of the fold quality was done with ERRAT (Colovos, et al., Protein Science 1993; 2:1511-1519).

Substrate suitability was approached by examining homologous proteins using the Uniprot Knowledgebase Release 15.2 sequence database. Substrates examined include 1,4-dihydroxy-2-naphthoate, oligoprenyl diphosphates, 4-hydroxybenzoate, 1,4-dihydroxy-naphthaline derivatives, and menaquinone (vitamin K-2). Substrate binding and dynamics (4-hydroxybenzoic acid and 1,4-naphthalin-diol) were evaluated using automated docking and molecular dynamics simulations (GOLD (Jones, et al., J Mol Biol 1997; 267:727-748)).

### B. Results And Discussion

1. Recruitment and Diagnosis of New SCD Families

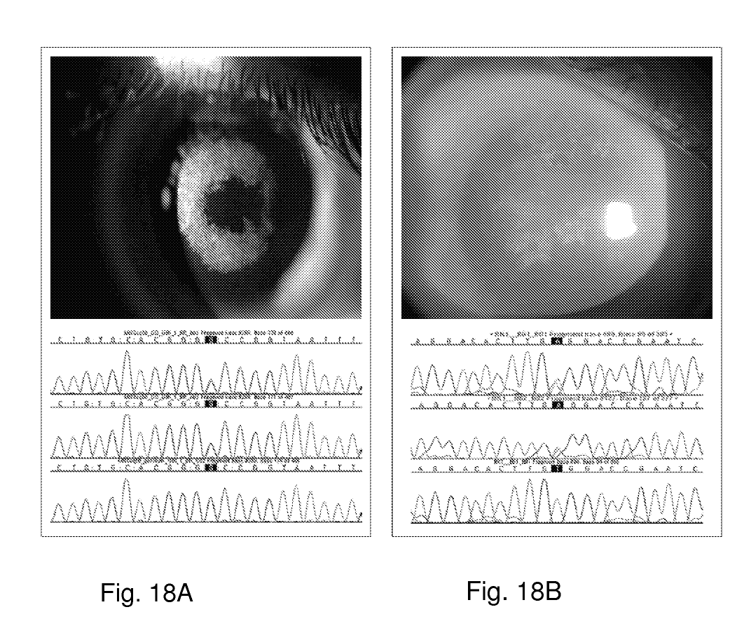

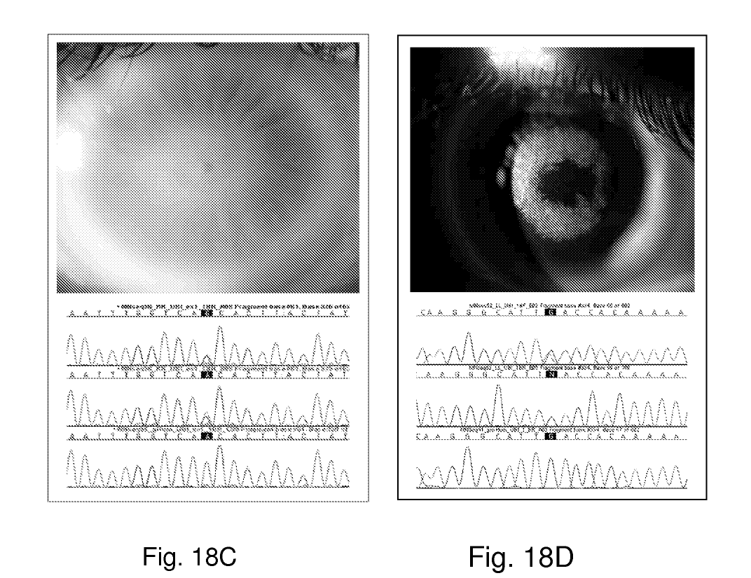

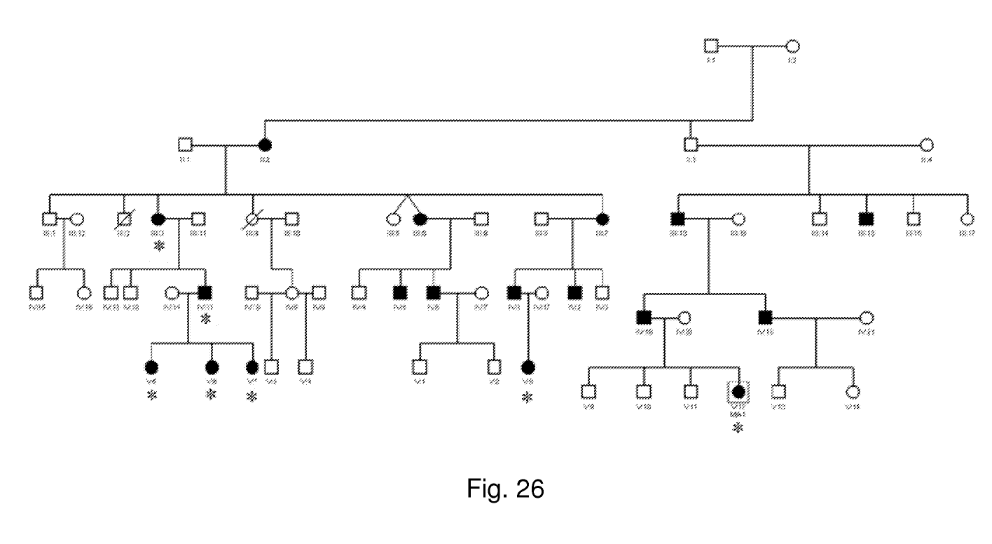

Ten affected individuals with SCD from different families were recruited as well as additional family members if possible. Six families resided in the United States, AA, GG, II, KK, LL, and MM. Four families resided out of the United States with Families CC from Japan, EE from Taiwan, N from Germany, and F1 from Finland. No known history of SCD was available in Families GG and LL and KK, II, and EE. There was documented family history of inherited corneal dystrophy in five families and three families had more than one affected individual participate in the study (Families AA, N, F1). Pedigrees of Families N and F1 are shown in Table 9 and FIG. 26. Affected patients demonstrated classical findings of SCD including superficial corneal crystals which appeared as central and paracentral deposits (FIG. 18A) in a 36 year old male proband from Family GG. Diffuse cornea haze with scattered superficial crystals and a peripheral arcus lipoides was demonstrated by a 69 year old male proband of Family AA (FIG. 18B) and represents a first report of SCD in a family of Native American ancestry. Central corneal opacity with superficial crystals, slight mid peripheral haze and prominent arcus lipoides were demonstrated by a 61 year old male proband in Family KK (FIG. 18C) of unknown ethnicity. FIG. 18D shows the cornea of a 25 year old male proband from Family LL with paracentral crystalline deposits.

Genetic analysis of the UBIAD1 gene was performed on probands representing ten new SCD families. Genetic details are shown in Table 9 (bold, ‘this report’ in Publication column) accompanied by previously characterized UBIAD1 mutations in published SCD families (Weiss Opthalmology 1996; 103: 465-473; Orr, et al., PLoS ONE 2007; 2(8): e685; Weiss, et al., Invest Opthalmol Vis Sci 2007; 48:5007-5012; Yellore, et al., Mol Vis 2007; 13:1777-1782; Weiss, et al., Am J Med Genet. 2008; 146A:271-283). FIGS. 18A-C (bottom) shows proband sequencing in UBIAD1 for Families GG, AA, KK, and LL leading to characterization of A97T, V122E, N102S, and D112N mutations in the protein, respectively. Five families exhibited novel mutations, A97T (Family GG), D112N (LL), V122E (AA), V122G (F1), and L188H (EE). Five newly recruited families possessed N102S ‘hotspot’ mutations: Families CC, II, KK, MM, and N. Family CC is of Japanese descent, Family N is from Germany, and Families MM, II, and KK reside in the United States but have uncertain ethnicity. Mutations of the N102 residue are shown as distinct in Table 9 but it should be noted that some families can be distantly related and share an N102S mutation due to a founder effect. Over 220 chromosomes from unrelated CEPH individuals were sequenced and examined at the site of each novel mutation. No alterations were found in these healthy individuals confirming that these mutations are likely associated with SCD and not rare polymorphisms. A discrepancy was noted in Family F1 where a patient was diagnosed as unaffected by corneal exam and carried a V122G mutation. This can be due to the young age of the patient (no specific age given by reviewing physician); it can also be due to the difficulties of making the diagnosis of SCD in some patients and/or families. (Weiss, et al., Invest Opthalmol Vis Sci 2007; 48:5007-5012). Additional blood samples will be collected from other members of this family to insure cosegregation of the disease with the observed mutation, and individual patients will be examined over time to assess expression of the corneal dystrophy phenotype. The family and mutation are included in this study as evidence strongly suggests this is a valid SCD alteration. The same UBIAD1 amino acid (V122) was observed to be mutated (V 122E) in the proband for Family AA and not in his unaffected sister indicating a critical role for mutation of this residue in disease. Lastly, examination of DNA from over 110 healthy individuals failed to indicate the presence of rare polymorphism(s) at this codon.

2. Homology across Species and Phylogeny of UBIAD1

In order to begin to assess how SCD mutations might affect protein function, conservation of the entire protein across species was examined. Previously, only putative active site residues were examined and residues mutated in SCD in Loops 2 and 3 were not examined. (Weiss, et al., Am J Med Genet. 2008; 146A:271-283). Protein sequences for UBIAD1 homologs were identified in 19 species, including human, chimp, canine, elephant, horse, hyrax, dolphin, megabat, guinea pig, mouse, rat, cat, opossum, chicken, zebrafish, medaka, sea urchin, sea anemone, and fruit fly. Protein homology was examined in FIG. 19 and an alignment generated using ClustalX (Larkin, et al., Bioinformatics 2007; 23: 2947-2948) across the full length protein sequence is shown in FIGS. 19A-B. Overall homology was very high when pairwise alignment scores were examined between UBIAD1 from selected species and human (NCBI HomoloGene 8336). Pairwise alignment scores for various mammals compared to the human protein ranged from 99.7% identity in chimps to 92.6% (mice), 91.7% (rat), 91.3% (cattle), and 89% (dog). Amino acid identity decreased in non-mammal vertebrates to 81.7% (chicken), 78.9% (zebrafish), and 59.6% (fruitfly).

Locations of 17 amino acids mutated in SCD patients (Table 9) are indicated in the alignment by arrows (FIG. 19A-B, top). Fifteen out of 17 residues were universally conserved in all 19 organisms examined from sea urchins to humans. The height of bars in the graph below the sequence alignment (grey) indicated the overall degree of conservation, i.e., the taller the bar, the higher the degree of conservation. Groups of SCD mutations (Loops 1, 2, and 3) are clustered in regions of high conservation, i.e., alignment positions-120-150 (Loop 1), 190-220 (Loop 2), and 245-263 (Loop 3). These are separated by regions of protein that are less conserved, i.e., residues in alignment positions 100-115, 160-180, and 230-240.

Conservation across species of amino acids corresponding to novel SCD mutations presented in this study is highlighted in FIG. 19C. Regions of alignment (FIGS. 19A-B) of UBIAD1 homologs from the species indicated (left) encompassing human SCD mutations: A97, D112, V122, L188, are shown. Locations of amino acids mutated in new SCD families are indicated. Two of four new SCD alterations were universally conserved across species from sea urchin to human. Two mutated residues were not completely conserved, A97 and L188. Alanine 97 conservation is disrupted only by the presence of a serine in sea anemone. The leucine residue 188 (alignment position 215) is conserved in all mammals but varies with valine in chicken, fish, and sea urchin, or an isoleucine (sea anemone). These substitutions for leucine appear to represent ‘allowable’ conservative substitutions between nonpolar, aliphatic residues. Based upon the alignment, a phylogenic tree was created (FIG. 19D). The tree is consistent with the high conservation of the protein in mammalian species and lesser but substantial conservation in vertebrates.

3. Linear and 2D Protein Models

A linear diagram of the UBIAD1 protein and a 2-D model of UBIAD1 in a lipid membrane are presented in FIGS. 20A and 20B, respectively, to show the numbers of independent families examined (by all publications to date) and locations of residues mutated in SCD. The linear model (adapted from Weiss, et al., Am J Med Genet. 2008; 146A:271-283) includes mutations from new families presented in this study (green arrows). The most N-terminal SCD alteration to date is reported, A97T, in Family GG of Irish-French Canadian ethnicity. Previously published familial mutations in putatively unrelated families are shown (black arrows). (Orr, et al., PLoS ONE 2007: 2(8):e685; Weiss, et al., Invest Opthalmol Vis Sci 2007; 48:5007-5012; Yellore, et al., Mol Vis 2007: 13:1777-1782; Weiss, et al., Am J Med Genet. 2008; 146A:271-283; Kobayashi, et al., Opthalmology 2009; 116:1029-1037). A mutation hotspot, the N102S alteration, is depicted by a column of 17 arrows near the first transmembrane spanning region. Seventeen of 41 (41%) SCD families exhibit this mutation, though a possibility exists that not all families with this alteration are unrelated. FIG. 20B shows locations of 17 different amino acids mutated in SCD in a 2D model of the protein in a lipid bilayer. Mutations appear to occur in clusters on regions of the protein lying towards one side of the membrane (Loops 1-3). Loop 1 of the protein appears to be affected by 9/10 new mutations reported here. New mutation, A97T, appears to extend the cluster of alterations in Loop 1 towards the N-terminus of the protein. Five additional families with a hotspot alteration, N102S, increase the significance of this Loop 1 residue, which was predicted to lie at the membrane-aqueous interface. D112N and two alterations at V122 (V122E and V122G) appear to affect more aqueous portions of Loop 1. A single Loop 2 mutation is L 188H, and is the most C-terminal mutation in this cluster. The alteration aligns with the G186R mutation in TM helix 4 to form a mirror image of two SCD alterations in neighboring TM helix 3, G177R and T1751.

4. Localization of UBIAD1 to Mitochondria in Keratocytes

To address the possibility that SCD mutations alter UBIAD1 protein trafficking, the subcellular localization of wild type and mutant human UBIAD1 was examined (FIG. 21). Localization within cultured normal human keratocytes of UBIAD1 and protein disulfide isomerase, an enzyme marker for the endoplasmic reticulum, is shown in FIGS. 21A-C. Co-localization of UBIAD1 and a subunit of OXPHOS complex I (NADH dehydrogenase), an enzyme in mitochondria, is shown in FIG. 21D-F. UBIAD1 labeling is red (FIGS. 21B and E), protein disulfide isomerase and OXPHOS I are green (FIGS. 21A and D). UBIAD1 did not co-localize with the endoplasmic reticulum (FIG. 21C), but did co-localize with mitochondria (co-localizing red and green show as orange in FIG. 21F). FIG. 22 presents localization of UBIAD1 in SCD and normal keratocytes. Keratocytes were cultured from surgically removed cornea from the proband of Family KK containing an N102S UBIAD1 alteration (Table 9). Co-localization of SCD mutant UBIAD1 protein and OXPHOS complex I mitochondrial marker in SCD disease keratocytes (FIGS. 22A-C) and normal human keratocytes (FIGS. 22D-F) is shown. UBIAD1 (red, FIGS. 22A and D) and the mitochondrial marker (green, FIGS. 22B and E) show co-localization (orange) in both normal (FIG. 22F) and SCD disease keratocytes (FIG. 22C).

5. Analysis of Cholesterol in SCD Patients

Mutation of UBIAD1 in SCD is thought to result in disregulation of cholesterol and lipid metabolism, which primarily exhibits as clinical symptoms affecting vision due to accumulation of cholesterol and lipids in patient corneas (Weiss, Trans Am Opthalmol Soc 2007; 105:616-648). Most SCD patients also exhibit hypercholesterolemia, though determining the significance of this association has been difficult due to the rarity of the disease and high frequency of use of cholesterol lowering drugs by SCD patients. Prior examination of homology between UBIAD1 and other proteins known to be involved in cholesterol metabolism (geranyl- and farnesyltransferases, for example) revealed UBIAD1 to be an outlier, and thus a potential novel component of cholesterol biosynthesis or homeostasis (Kobayashi, et al., Opthalmology 2009; 116:1029-1037). To assess the impact of UBIAD1 on cholesterol metabolism, several forms of cholesterol were examined in cell lines from wild type (non-SCD) and SCD patients expressing wild type and mutant protein.

B cell lines were established from male probands of two SCD families (AA and GG). Family AA represents the first SCD family of Native American ethnicity and the proband, but not his unaffected sister, exhibited a V122E mutation. The Family GG proband self-reported an ethnicity of Irish and French Canadian and possessed an A97T UBIAD1 mutation (Table 9 and FIG. 1). B cell line pellets from each probands, an unaffected sister from Family AA, and three healthy donors were analyzed for protein and cholesterol (Table 10). No significant differences were observed in total cholesterol, cholesteryl ester, and unesterified cholesterol in SCD and healthy patient B cell lines.

6. Protein Threading Model of UBIAD1

In order to examine UBIAD1 structure-function relationships in more detail, particularly with regards to the potential impact of SCD mutations, additional three dimensional (3D) modeling was performed. No useful templates for a model could be identified from a search of the Brookhaven Protein Data Bank using BLASTp. Proteins homologous to UBIAD1 with at least 30% identity in the amino acid sequence and with a resolved X-ray structure were sought. An additional search was performed using PHYRE that included sequence alignment, secondary structure prediction, and alignment of secondary structure elements. Two templates belonging to the MFS general substrate transporter family were identified as somewhat similar but were not used for additional modeling due to differences in arrangement of TM helices: a metal transporter and an ABC transporter (pdb-codes: 2cfg and 1qw4, respectively).

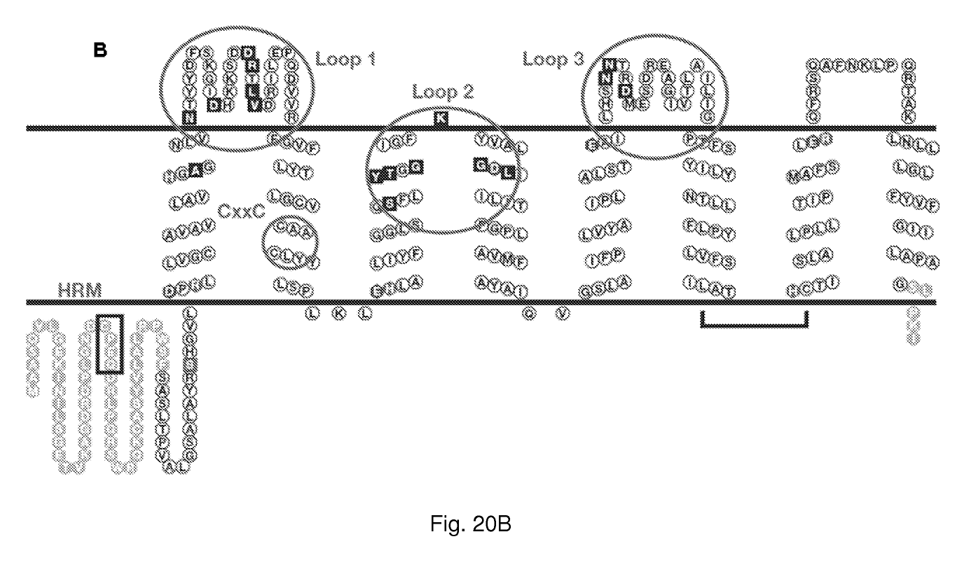

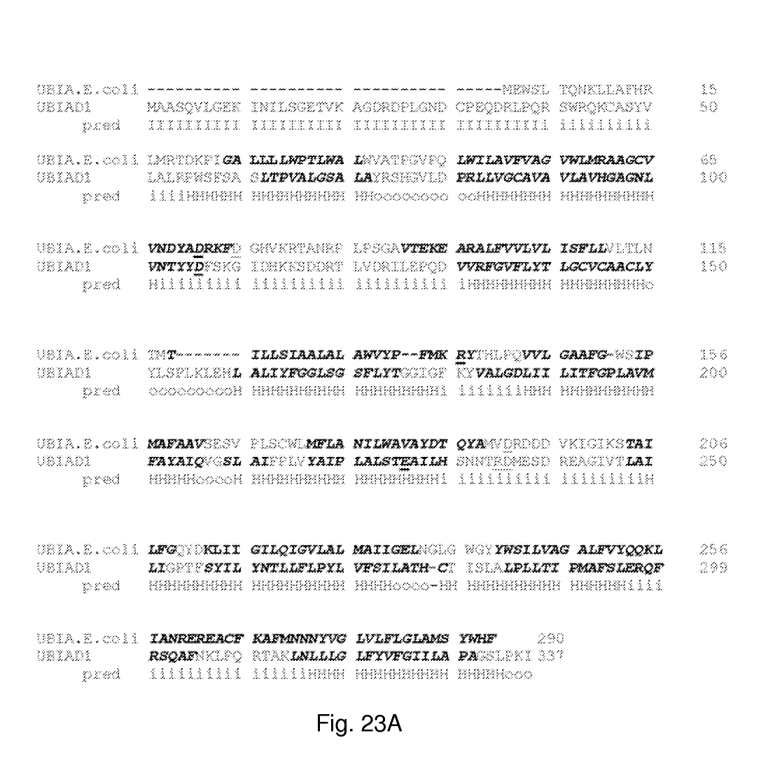

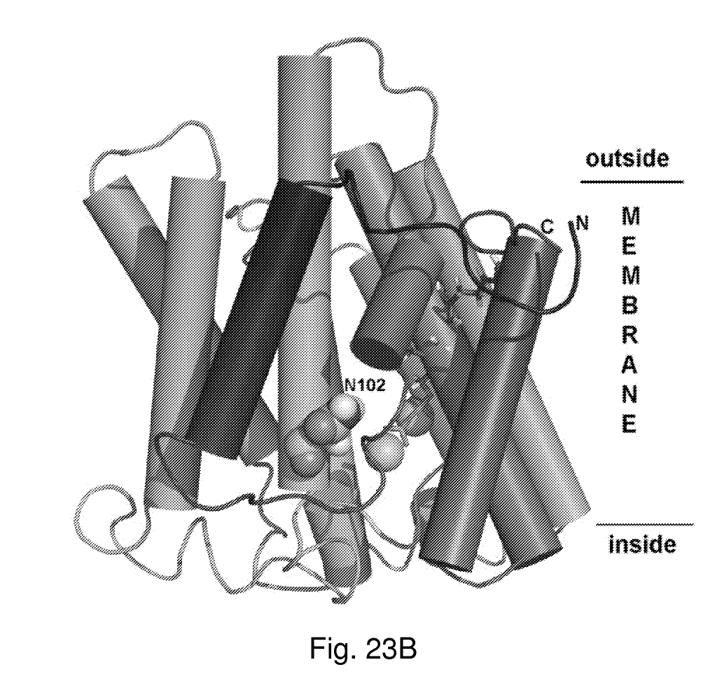

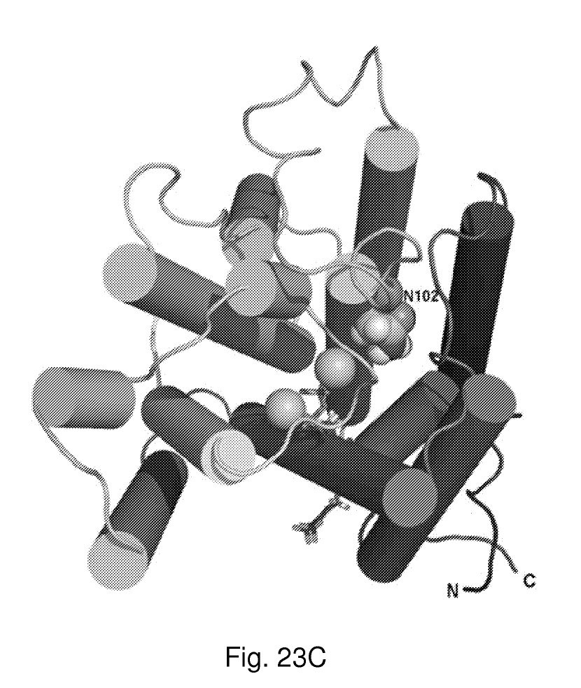

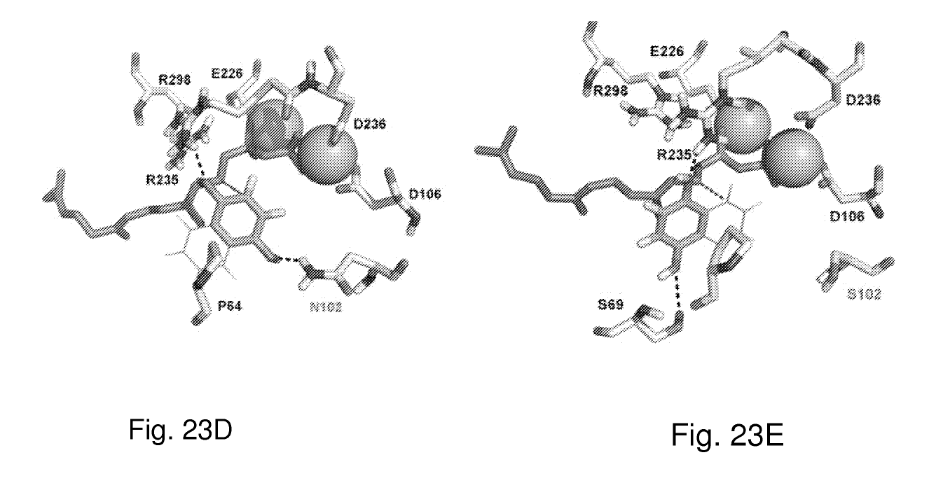

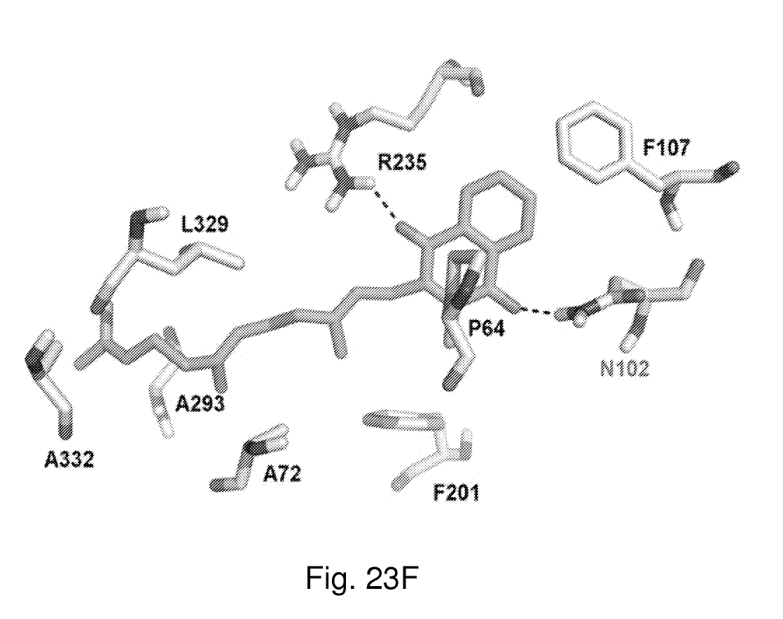

Available X-ray structures of prenyl-converting enzymes were examined as templates, such as prenyl synthases (cyclases), protein prenyl transferases, and the recently developed model of the all-alpha-helical E. coli UbiA (Bräuer, et al., Chembiochem 2008; 9:982-992; Wessjohann, et al., Angew Chem Int Ed Engl 1996; 35:1697-1699). Modeling using the Molecular Operating Environment (MOE) indicated UbiA but not other proteins examined possessed an arrangement of alpha helical structural elements that could be superimposed on UBIAD1 (FIG. 23A). The positional placement of geranylpyrophosphate and a single magnesium cation were extracted from the E. coli UbiA model and fitted into the model of UBIAD1. A second magnesium cation was manually added to the model due to an additional aspartate close to the putative binding site of the pyrophosphate moiety in UBIAD1. Two or even three magnesium ions are not uncommon in diphosphate binding and activation.

The model obtained from MOE was refined by the molecular dynamics refinement tool YASARA. Analysis with PROCHECK for stereochemical quality indicated that 86.7% of all amino acid residues were located in the most favored area and only three residues were in disallowed (uncertain) loop regions. All parameters evaluated by PROCHECK are better (overall G-factor) than similar values for an analogous X-ray crystal structure at a 2 Å resolution. Inspection of the fold quality was done with ERRAT and revealed a quality indication of 94%, with low quality scores in only five small regions.

Over 30 models were generated and evaluated during the analysis to obtain the model shown in FIG. 23B (side view) and 23C (top view). The 3D-protein model of UBIAD1 based on alignment with UbiA (FIG. 23A) fits best when compared to several other templates including protein prenyltransferases, terpene synthases, and oligoprenyl synthases. Transmembrane helices are shown in FIGS. 23B and 23C as rainbow colors in an approximate circular pattern to form a central binding area on one side of the membrane. The approximate location of the lipid bilayer is indicated (horizontal lines, FIG. 23B). Inside and outside are arbitrary labels of membrane sidedness and can be interchanged. The side chain of the SCD mutation hotspot residue, N102, is shown (spacefill atom) to occupy a position where the first TM helix exits the membrane (likely towards the inside). A top view (FIG. 23C) shows the spacefill N102 sidechain to point inwards towards the center of a putative prenyldiphosphate binding pocket. Green spheres represent magnesium cations in the active site with a docked farnesyldiphosphate (red stick representation). The prenyl substrate appears to approach the active site containing N102 from the central cavity.

The model allowed predictions to be tested about possible ligand(s)/substrate(s) of UBIAD1. As a basis for comparison, the enzymatic reaction catalyzed by UbiA is shown in Figure S3 (Bräuer, et al., Chembiochem 2008; 9:982-992; Bräuer, et al., J Mol Model 2004; 10:317-327). A search of the UniProt Knowledgebase, Release 15.2, sequence database was performed. Based on sequence homology, the most similar protein, in addition to proteins related to UbiA, is 1,4-dihydroxy-2-naphthoate octaprenyltransferases (e.g. from Aedes aegypti, entry code: Q17BA9_AEDAE). Based upon this homology, substrates similar to oligoprenyl diphosphates were examined. These appear likely candidates as the model possessed a matching binding pocket. UBIAD1 appears to be related to aromatic prenyltransferases and, similar to these enzymes, a second substrate can be involved in catalysis, e.g. 4-hydroxybenzoate (cf. UbiA) or a 1,4-dihydroxy-naphthaline derivative (cf. Q17BA9_AEDAE). For the latter case, in bacteria, menaquinone (vitamin K-2) is the product of such a prenylation reaction, a farnesylation in position 3 (which equals position 2 in unsubstituted 1,4-naphthalin-diol) of the aromatic substrate 2-methyl-1,4-naphthohydroquinone. This molecule is structurally similar to 1,4-dihydroxy-2-naphthoate. Both 4-hydroxybenzoic acid and 1,4-naphthalin-diol as core elements of speculative second substrates of UBIAD1 were successfully docked into the putative active site of the model using GOLD. The latter second substrate docking simulation is shown in Figure S4. (Bräuer, et al., Chembiochem 2008; 9:982-992; Bräuer, et al., J Mol Model 2004; 10: 317-327). A tertiary protein structure model of human UBIAD1 with eight TM helices is shown with a putative naphthalinediol substrate that docks preferentially in the central cavity near the active site residue, N102.

In an attempt to explore the similarity between UBIAD1 and somewhat homologous aromatic prenyltransferases, unbiased docking simulations of substrates (farnesyldiphosphate and a 1,4-dihydroxy aryl compound) were conducted with models of wild type (N102) and SCD mutant (S102) UBIAD1 (FIGS. 23D and 23E, respectively). As shown, a substrate diphosphate binding site was identified in the putative active site. (Weiss, et al., Am J Med Genet. 2008; 146A:271-283) The most frequently mutated residue in SCD (41% of families, Table 9), N102, is an integral part of this putative active site. In this docking simulation, the N102 residue formed weak hydrogen bonds to the 1,4-dihydroxy aryl compound (dotted line) which were lost upon mutation to a serine residue. At this point, it is not clear whether UBIAD1 serves an enzymatic or other function. Of the many prenylated aromatics which serve critical functions in human metabolism, few are actually synthesized in humans. Several are supplied by external sources as vitamins, such as tocopherols (vitamins E) and phylloquinones (vitamins K, e.g. menaquinone). Thus prenylated aromatics with a role in human metabolism which are not likely substrates but can be ligands were also docked to the model. Results show that menaquinone fits excellently into the interior of ubiAD1 models (FIG. 23F).

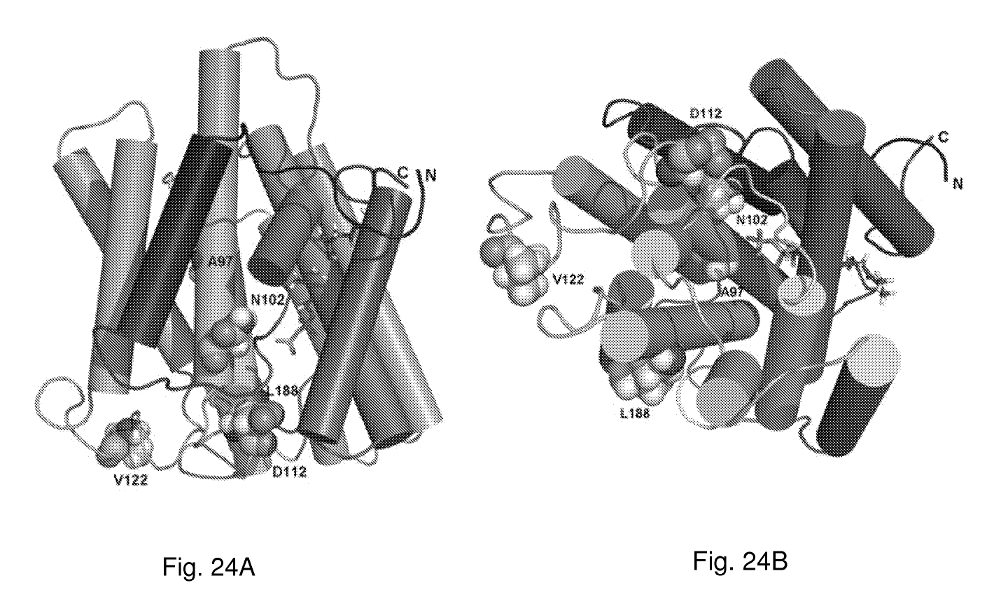

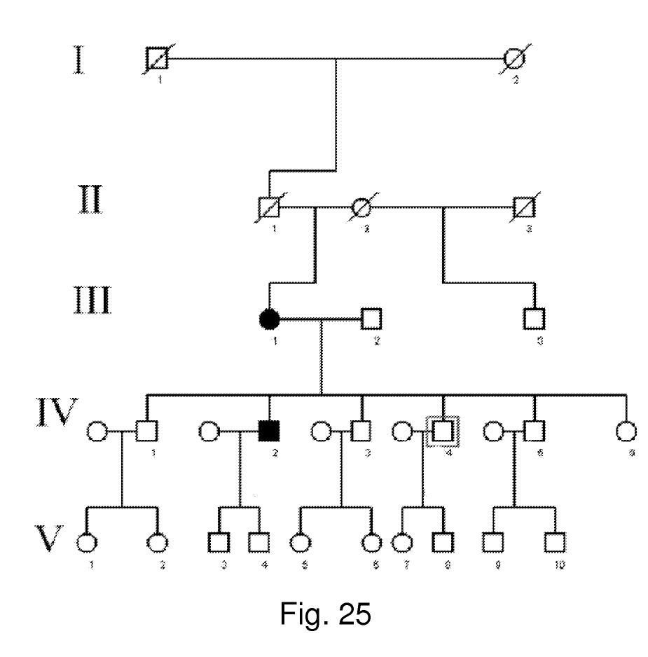

Other amino acids mutated in SCD were found in the vicinity of the active site and selected residues (A97, N102, D112, V122, L188) are presented in FIGS. 24A and B. FIG. 24A shows a side view of the model with novel and hotspot SCD mutations described in this publication labeled. A top view of the model is shown in FIG. 24B. SCD mutations appear to cluster in three areas of the protein, labeled Loops 1, 2, and 3, in FIG. 20B. The loops were highlighted (see Figure S4, Bräuer, et al., Chembiochem 2008; 9:982-992; Bräuer, et al., J Mol Model 2004; 10: 317-327). Loop 1 (A97 to R132) is shown in orange, loop 2 (Y174 to A184) in blue, and loop 3 (L229 to S257) in green. Mutated S102 is shown as a spacefill atom and included in the figure is a docked farnesyldiphosphate (shown as a stick representation in red). Significantly, a previously described polymorphism, S75F, (Orr, et al., (2007) PLoS ONE 2: e685; Weiss, et al., Invest Opthalmol Vis Sci 2007; 48:5007-5012) was not identified as functionally important to entry of substrates or substrate catalysis.

Recruitment of new families and individuals with SCD facilitates investigation of the genotypic and phenotypic spectrum of this disease. Photos of corneal manifestations of SCD (FIGS. 18A-D) highlight the phenotypic diversity of the disease. Of ten new families described in this example, five possessed novel UBIAD1 alterations. The A97T mutation detected in Family GG is the most N-terminal mutation yet characterized and extends the Loop 1 mutation cluster toward the middle of TM helix 1 (FIG. 20B). Two different mutations documented at the V122 residue (V122E in Family AA and V122G in Family F1) extend Loop 1 toward the C-terminus by one amino acid. Lastly, the L188H mutation in Family EE expands Loop 2. Mutations in Loop 2 appear to form a pattern on both sides of TM helices three and four potentially indicating critical function for this hydrophobic region of the protein. All five of the novel amino acid substitutions presented here represent non-conservative changes: nonpolar to polar (A97T), negative amino acid to neutral (D112N), nonpolar to polar and negative (V122E), aliphatic to non-aliphatic (V122G), and nonpolar to polar and positive (L188H). These novel mutations are consistent with previously published alterations in this regards (Table 9).

A summary of all SCCD mutations published to date indicates three amino acids are mutated to multiple residues: aspartic acid 112 to either an asparagine or a glycine, leucine 121 to a valine or a phenylalanine, and valine 122 to a glutamic acid or a glycine. Examining alteration of valine 122, for example, it appears that SCD results from either substitution of valine (non-polar) with a polar, negative amino acid (glutamic acid) or a non-polar, neutral one (glycine). Loss of valine 122 rather than gain of a specific mutant residue appears to be critical for SCD. These cases appear to indicate that loss of key amino acids is critical for SCD rather than a gain of function due to mutation.

The high degree of amino acid identity in this protein that is conserved across species indicates that it can have an important metabolic function that is essential. These function(s) can play a role outside the visual system as the gene is widely expressed in human tissues (McGarvey, et al., Oncogene 2001; 20:1042-1051) and is highly conserved in the megabat (FIG. 19). Additionally, 15/17 amino acids mutated in SCD are conserved in all organisms examined, including sea urchin and sea anemone. Aromatic prenylation, which is evolutionary at least as old as aerobic life, is known but not very common in human metabolism. Accordingly, UBIAD1 can have a common origin with/from E. coli UbiA, but can not necessarily act as a transferase (See Figure S3, Bräuer, et al., Chembiochem 2008; 9:982-992; Bräuer, et al., J Mol Model 2004; 10: 317-327). The protein can function equally well as a sensor, receptor (signal transduction), or pore protein. The protein can utilize any combination of the residual binding domains for prenyl chains, diphosphate, or phenolic compounds for this purpose. Additional protein-protein interaction data (McGarvey, et al., J Cell Biochem 2005; 95:419-428) and heterozygous effects strengthen a functional role as a putative receptor for sensing and/or signaling, e.g. connected to oligoprenyl-related cholesterol metabolism.

The alignment of UbiA from E. coli, human UBIAD1 and a prediction of transmembrane helices is shown in FIG. 23A. The alignment reasonably explains both the eight membrane helices in UBIAD1 as well as its relation to UbiA. The 3D protein model of UBIAD1 based on UbiA and this alignment (FIGS. 23B-C) fits best, compared to several other templates tested including protein prenyltransferases, terpene synthases, and oligoprenyl synthases (Brandt, et al., Phytochemistry 2009; 70(15-16):1758-1775. In the model, a diphosphate binding site can be identified and appears to be an integral part of a putative active site (Weiss, et al., Am J Med Genet. 2008; 146A:271-283) (FIGS. 23D-E). The most frequent SCD mutation, N102S, was observed to be an integral part of this putative active site. Other relevant SCD mutations (FIG. 24) were found in the vicinity of the active site, while a polymorphism found in approximately 3% of healthy individuals, S75F (Orr, et al., PLoS ONE 2007; 2(8):e685; Weiss, et al., Invest Opthalmol Vis Sci 2007; 48:5007-5012; Weiss, et al., Am J Med Genet. 2008; 146A:271-283) did not appear to be functionally important, i.e., near the putative active site or sites of SCD mutation.

The usefulness of the protein model presented here relies on its ability to allow structure-function predictions that can be examined experimentally. Potential shortcomings are clear, namely low overall sequence homology between E. coli UbiA and human UBIAD1, and using a model (of UbiA) to create another model (UBIAD1). Low homology, however, appears to be common throughout this group of all alpha-helical prenyldiphosphate converting enzymes, which nevertheless give convergent results using threading. Regarding apparent low homology between the E. coli and human proteins, examination of individual residues critical for UbiA enzyme function can be informative with regards to UBIAD1 and SCD. Five UbiA amino acids were judged as crucial for catalytic activity based upon pre-UbiA 3D modeling studies: Asp71, Asp75, Arg137, Asp191, and Asp195. (Bräuer, et al., Chembiochem 2008; 9:982-992; Bräuer, et al., J Mol Model 2004; 10: 317-327). These amino acids were individually mutated to alanine and UbiA enzyme function was measured by examining conversion of geranyl diphosphate to geranyl hydroxybenzoate (geraniol) in a standard assay (Wessjohann, et al., (2009) Chimia and Phytochemistry, in press; Momose, et al., J Biol Chem 1972; 247:3930-3940). Four of five mutations inhibited product formation completely and R137A reduced activity by approximately 95%. (Bräuer, et al., Chembiochem 2008; 9:982-992).

Modeling of UbiA connected these decreases in enzyme activity to specific chemical functions, i.e., activation of a phenolate intermediate by Asp191 binding of p-hydroxybenzoate, diphosphate salt bridge formation by Arg137, and stabilization of the Arg137 side chain by Asp195. Arg137 was predicted to play a key role in prenyl substrate recognition and positioning relative to the hydroxybenzoate substrate.

FIG. 23A compares human UBIAD1 with E. coli UbiA based upon alpha helical structural alignment (see Methods). Two of the key E. coli residues (Arg137 and Asp191) align with corresponding human protein amino acids that are mutated to cause SCD. E. coli Arg137 corresponds to UBIAD1 Lys181 (Kobayashi, et al., Opthalmology 2009; 116:1029-1037) which is mutated to an arginine in SCD (Family: Case 4, Table 9 (Weiss, et al., Am J Med Genet. 2008; 146A:271-283)). Asp191 in UbiA corresponds to UBIAD1 Asp236 (Family: FF, Table 9 (Weiss, et al., Invest Opthalmol Vis Sci 2007; 48:5007-5012)). This amino acid is the most C-terminal SCD mutation characterized to date and appears in the Loop 3 cluster in an aqueous portion of the protein. In SCD, Asp236 is mutated to glutamic acid. UbiA Asp71, 75, and 195 correspond to UBIAD1 Asp106, Gly110, and Asp240 in the alignment. Two of three residues are conserved. It is unlikely that UbiA Asp75 function is maintained by human Gly110 as shown in the alignment but nearby is an asparagine, human Asp112, that is mutated to either an asparagine (SCD Family LL, this report) or a glycine (SCD Family F122 (Orr, et al., PLoS ONE 2007; 2(8):e685)). The alignment also shows that several UBIAD1 residues mutated in SCD are conserved in UbiA, perhaps suggesting additional functional links between the enzymes that can be experimentally verified using site-directed mutagenesis of UbiA and the UbiA enzyme assay. For example, UBIAD1 Ala97 and Asn102 correspond to UbiA Ala62 and Asn67. Mutagenesis of these residues in UbiA might show loss of activity. Thus, SCD in humans appears to indicate additional amino acid residues can be critical for UbiA (correct) function. A concluding point is that the two UbiA residues (Arg137 and Asp 191) were mutated to alanine and enzyme function was lost (Bräuer, et al., Chembiochem 2008; 9:982-992). These two residues align with amino acids mutated in humans to cause SCD, Lys181 and Asp236. If the correlation holds, this appears to provide a second indication that SCD can result from loss of activity of UBIAD1.

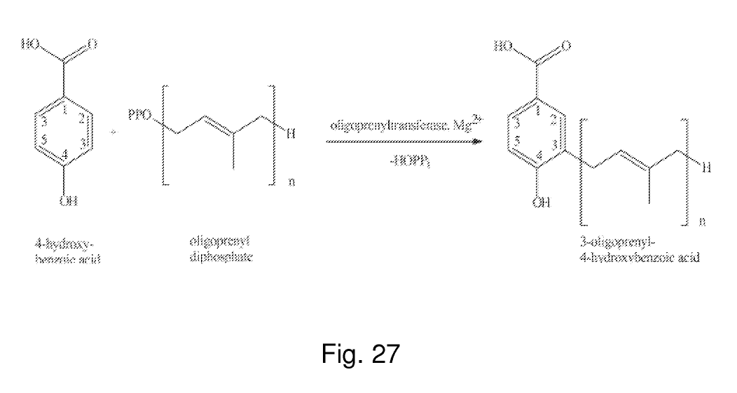

The 3D protein model clearly shows an optimal binding pocket for types of compounds such as oligoprenyl diphosphates (FIGS. 23B and C). UBIAD1 can be an aromatic prenyl transferase as indicated by its closest known protein homologues (see Results). If so, a second substrate or ligand moiety can be involved in enzyme catalysis, e.g. 4-hydroxy benzoate (cf. UbiA (Bräuer, et al., Chembiochem 2008; 9:982-992; Bräuer, et al., J Mol Model 2004; 10:317-327) and FIG. 27) or a 1,4-dihydroxy-naphthaline derivative (cf. 1,4-dihydroxy-2-naphthoate octaprenyltransferase). In bacteria, menaquinone (vitamin K-2) is the product of such a prenylation, a farnesylation at position 3 of the aromatic substrate 2-methyl-1,4-naphthohydroquinone, which is structurally similar to 1,4-dihydroxy-2-naphthoate. Naphthalin-1,4-diol as core element of such a speculative second substrate of UBIAD1 was docked into the putative active site of the model and fits very well (FIG. 28). Although detailed modeling of active site residues is full of uncertainty, changing N102 to N102S, as in SCD, changes the binding of this second substrate completely in the model, rendering its prenylation at position 3 impossible (FIGS. 23D and E). This is a third line of evidence that mutation of UBIAD1 in SCD can lead to a loss of function of the protein/enzyme.

The same is true if binding of a native ligand to UBIAD1 is not comprised of a prenyldiphosphate plus aromatic substrate (enzyme variant), but of a product-like prenylated aromatic (receptor/pore variant). Prenylated aromatics have many functions in human metabolism, but few of are actually synthesized in humans. Many are supplied by external sources as vitamins such as tocopherols (vitamin E) and phylloquinones (vitamin K, e.g. menaquinone) which are important for physiological processes including free radical protection and blood coagulation, respectively. Menaquinone fits excellently into the interior of UBIAD1 models (FIG. 23F). Since a relationship between menaquinone and cholesterol metabolism has been suggested by prior publications, (Shirakawa, et al., Biochimica et Biophysica Acta 2006; 1760:1482-1488) a hypothesis can be envisioned that links UBIAD1, perhaps via menaquinone sensing or transport, to cholesterol metabolism, which in turn has relevance for SCD.

FIG. 24 shows that all SCD mutations are in the loops of the protein located on one side of the membrane and all appear to be directly connected or in close proximity to the putative binding (or active) site domain. The SCD mutation hotspot, N102, in particular, is directly pointing into what is assumed to be the binding/action cleft. All SCD mutations are located on the same side of the membrane and appear to be oriented in or near the putative oligoprenyl diphosphate recognition site in such a way that they can either have a gatekeeper function for substrates or can influence protein-protein interaction(s). Though the model shows that mutation clusters are close to the entrance, these protein loops are usually flexible and the positions of SCD mutation clusters can be different than shown in the model, e.g., they can bend more towards the potential substrate entrance (FIG. 24B). Loop folding is generally less predictable by the modeling methods applied here. SCD mutations in Loops 1-3 can mediate interactions with one or more proteins, for example apolipoprotein E (McGarvey, et al., J Cell Biochem 2005; 95:419-428). These interactions can be modified by substrate binding or, conversely, protein-protein interactions can modify gate-keeper function(s) of the loops. As apolipoprotein E is the only protein to be experimentally verified as interacting with UBIAD1, it will be interesting to examine this interaction in greater detail using the model presented here and with expression constructs incorporating UBIAD1 transcripts containing SCD alterations.

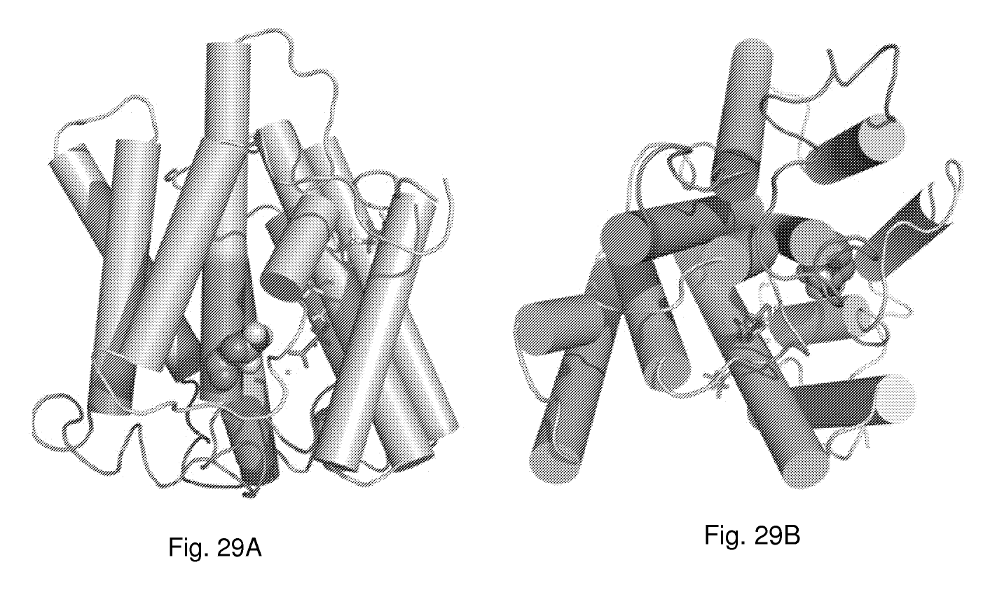

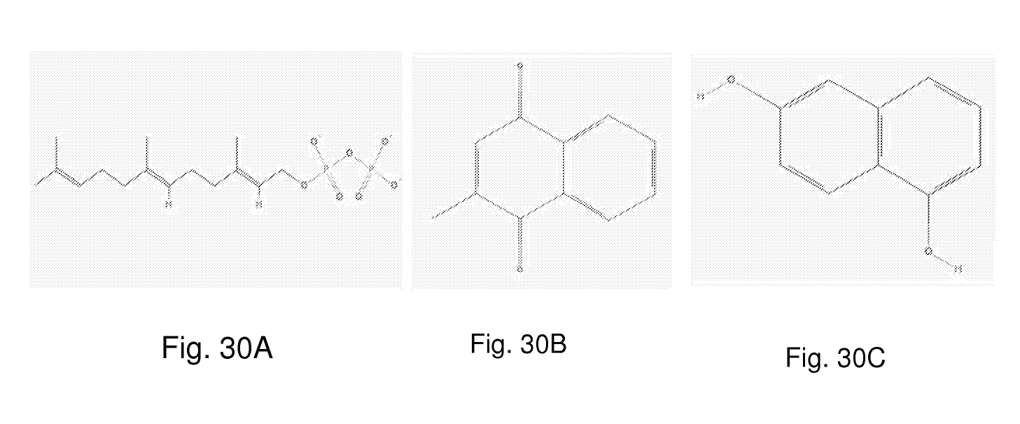

In order to expand a view of a cellular role for UBIAD1, localization of wild type and mutant protein was examined in keratocytes (FIGS. 28 and 29). FIG. 21 shows that UBIAD1 did not localize with a marker for endoplasmic reticulum (protein disulfide isomerase). UBIAD1 did demonstrate co-localization with a mitochondrial marker, OXPHOS complex I (FIG. 21F). Keratocytes were cultured from the Family KK proband (N102S UBIAD1 mutation) undergoing surgical removal of opacified cornea and were subjected to immunohistochemistry. As in FIG. 21, wild type UBIAD1 exhibited co-localization with a mitochondrial marker. The same results were observed when mutant UBIAD1 was examined in patient-derived keratocyte cells. This provides a preliminary indication that mis-localization of the protein is not a result of UBIAD1 mutation (at least for N102S). Mitochondrial localization is surprising in light of a previous report demonstrating interaction between UBIAD1/TERE1 and apolipoprotein E (McGarvey, et al., J Cell Biochem 2005; 95: 419-428). To our knowledge, a mitochondrial localization for apolipoprotein E has not been reported in the literature. However, some UBIAD1 immunostaining was localized outside of mitochondria making interaction with apoE a possibility. Experiments are underway to clarify where the interaction occurs between wild type proteins, for example in the presence of apolipoprotein E the localization of UBIAD1 can change; and whether the interaction occurs between wild type apolipoprotein E and SCD mutant UBIAD1.

Lastly, SCD has been associated with deregulation of cholesterol metabolism in the cornea and can potentially play a role in hypercholesterolemia (Rodrigues, et al., Am J Opthalmol 1987; 104:157-163). Separately, UBIAD1/TERE1 was shown to interact with apolipoprotein E (McGarvey, et al., J Cell Biochem 2005; 95: 419-428). UBIAD1 has been shown to be expressed in B cells (see FIG. 19 (McGarvey, et al., Oncogene 2001; 20:1042-1051) and the Expressed Sequence Tag database), so it was examined to determine whether alteration in cholesterol could be detected in B cell lines established from SCD patient blood samples, an unaffected sibling, and healthy volunteers. Total cholesterol, cholesteryl ester, and unesterified cholesterol were measured (Table 10). Probands from two SCD families (Family AA and GG with V122E and A97T UBIAD1 mutations, respectively) were examined. No significant differences in cholesterol content were observed in SCD and healthy patient B cell lines under the conditions examined. Additional analyses can be performed of protein-protein interactions (UBIAD1 and apolipoprotein E (McGarvey, et al., J Cell Biochem 2005; 95:419-428)) and potential post-translational modification of protein binding partners, such as with a cholesterol or cholesterol-like moiety (Breitling, BioEssays 2007; 29:1085-1094).

Presently, the only treatment for SCD is corneal replacement by PKP once corneal opacification causes visual decrease.

PKP is performed in the majority of patients above the age of 50 years with SCD (Weiss, Trans Am Opthalmol Soc 2007; 105:616-648). Unfortunately, there are no present therapies to prevent the progressive lipid deposition in the cornea which results in this visual loss.

Prior studies have demonstrated that normalizing blood cholesterol levels does not affect the relentless deposition of corneal lipid that occurs with age (Lisch, et al., Ophthalmic Paediatr Genet. 1986; 7,45-56). Hopefully further understanding about the impact of UBIAD1 mutations in SCD will potentially lead to interventional strategies to prevent visual loss in these patients. It appears from results presented above that UBIAD1 function is lost or decreased by SCD mutations. Thus, therapeutic analogs of substrates which were successfully docked to the UBIAD1 model (FIG. 23) can further inhibit rather than restore protein function. Examination of protein binding partners can allow useful therapeutic targets to be identified.

TABLE 9
Summary of Mutations in SCD Families^{a}

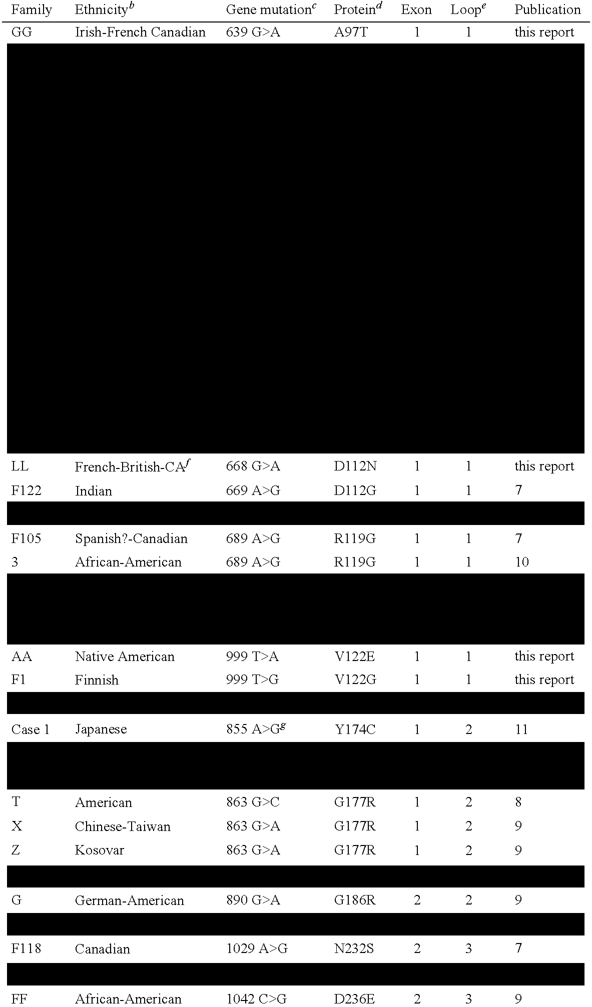

^{a}Desccending order 5′ to 3′ nucleotide number in the Reference Sequence. Alternating shading by sequential mutated amino acid.
^{b}Ethnicity is given if known, otherwise location of proband is listed.
^{c}location of mutation in RefSeq NM_013319.2 .
^{d}Predicted effect of genetic mutation on protein NP_037451.
^{e}Loop, see FIG. 39.
^{f}CA, Canadian Native American.
^{g}Nucleotides re-numbered based upon updated RefSeq NM_013319.2.

TABLE 10

| Patient B cell line | | TC (nmol/mg pr.)^{a} | UC (nmol/mg pr.)^{b} | CE (nmol/mg pr.)^{c} | Ester/Total (%) |
|---|---|---|---|---|---|
| ID | SCD status | AVE ± SD | AVE ± SD | AVE ± SD | AVE ± SD |
| 66K00597 | unaffected control | 44.0 ± 8.1 | 44.5 ± 8.7 | −0.6 ± 2.2 | −1.2 ± 4.5 |
| BUC692RDP692A | | | | | |
| 66K00594 | unaffected control | 51.7 ± 8.4 | 49.9 ± 8.6 | 1.8 ± 0.2 | 3.5 ± 0.9 |
| BUC704RDP704A | | | | | |
| 66K00595 | unaffected control | 57.5 ± 0.3 | 55.1 ± 0.8 | 2.4 ± 1.0 | 4.2 ± 1.8 |
| BUC708RDP708A | | | | | |
| 64101374 | affected proband | 44.1 ± 0.7 | 42.2 ± 1.3 | 1.8 ± 0.6 | 4.2 ± 1.4 |
| | SCD Family AA | | | | |
| 64101375 | unaffected sibling | 46.9 ± 2.3 | 45.9 ± 2.2 | 1.0 ± 0.2 | 2.2 ± 0.4 |
| | SCD Family AA | | | | |
| 64101376 | affected proband | 51.1 ± 1.5 | 49.6 ± 1.2 | 1.5 ± 0.3 | 3.0 ± 0.5 |
| | SCD Family GG | | | | |

^{a}TC: Total cholesterol
^{b}UC: unesterified cholesterol
^{c}CE: cholesteryl ester

The references cited herein, are all incorporated by reference herein, whether specifically incorporated or not.

Having now fully described this disclosure, it will be appreciated by those skilled in the art that the same can be performed within a wide range of equivalent parameters, concentrations, and conditions without departing from the spirit and scope of the present disclosure and without undue experimentation.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods disclosed herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are disclosed herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically disclosed herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. An isolated polynucleotide having the nucleotide sequence of or which is complementary to at least a portion of the UBIAD1 gene, wherein said nucleotide sequence contains at least one gene mutation which correlates with the risk of Schnyder's crystalline corneal dystrophy (SCCD) and wherein the at least one gene mutation is located at the codon corresponding to amino acid position 97, 118, 121, 122, 171, 177, 186, 188, 236 or 240 of SEQ ID NO:2, and wherein the gene mutation causes a change in the amino acid encoded by that codon, with the proviso that the codon corresponding to amino acid position 121 of SEQ ID NO:2 does not encode valine.

2. The isolated polynucleotide of claim 1 wherein the change in the amino acid is a nonconservative change.

3. The isolated polynucleotide of claim 1 wherein the polynucleotide is labeled with a detectable agent.

4. The isolated polynucleotide of claim 1 wherein the polynucleotide comprises between 10 and 40 consecutive nucleotides.

5. The isolated polynucleotide of claim 1 wherein the gene mutation results in a Ala97Thr, Asp118Gly, Leu121Phe, Val122Gly, Val122Glu, Ser171Pro, Gly177Arg, Gly186Arg, Leu188His, Asp236Glu, or Asp240Asn substitution.

6. A method for determining the presence or absence of one or more gene mutations of the UBIAD1 gene of SEQ ID NO:1 comprising the steps of:
a. obtaining a biological sample from the subject;
b. determining the presence or absence of one or more gene mutations of the UBIAD1 gene of SEQ ID NO:1 wherein the at least one gene mutation is located at the codon corresponding to amino acid position 97, 118, 121, 122, 171, 177, 186, 188, 236 or 240 of SEQ ID NO:2; and
c. determining if the gene mutation results in a change in the amino acid wherein the presence of the one or more gene mutations resulting in the change in the amino acid indicates the presence of the risk factor for a disease and/or the disease.

7. The method of claim 6 wherein the change the in amino acid is a non-conservative change.

8. The method of claim 6 wherein the determining of the presence or absence of the gene mutation further comprises the step of amplification of at least a portion of the nucleic acid using one or more pairs of oligonucleotide primers flanking at least one of the codons corresponding to amino acid position b 97, 118, 121, 122, 171, 177, 186, 236 or 240.

9. The method of claim 6 wherein the gene mutation results in a Ala97Thr, Asp118Gly, Leu121Phe, Val122Gly, Val122Glu, Ser171Pro, Gly177Arg, Gly186Arg, Leu188His, Asp236Glu, or Asp240Asn substitution.

10. (canceled)

11. A method of screening for an effect of a mutation in the UBIAD1 gene in cholesterol metabolism comprising:
a. providing a first aliquot of a purified protein which is involved in cholesterol metabolism;
b. contacting the first aliquot of purified protein with a non-mutant protein encoded by the UBIAD1 gene of SEQ ID NO:1;
c. determining the amount of the non-mutant protein that is bound to the purified protein;
d. contacting a second aliquot of the purified protein with a mutant protein encoded by a mutant UBIAD1 gene;
e. determining the amount of mutant protein encoded by the mutant protein that is bound to the purified protein; and
f. comparing the amount of non-mutant protein bound to the purified protein with the amount of the mutant protein bound to the purified protein wherein a difference in the amounts indicates that the mutation in the UBIAD1 may be is involved in cholesterol metabolism.

12. The method of claim 11 wherein the protein involved in cholesterol metabolism is apolipoprotein A-I, apolipoprotein A-II, apolipoprotein E, apolipoprotein B, or HMG-CoA reductase.

13. The method of claim 11, wherein the screening is performed to determine the presence of a risk factor for atherosclerosis.

14. The method of claim 11, wherein the screening is performed to determine the presence of atherosclerosis.

15.-17. (canceled)

18. The method of claim 6 wherein the method is used for diagnosing SCCD in a subject.

19. The method of claim 6 wherein the method is used for determining whether a subject is at risk for developing atherosclerosis.

20. The method of claim 6 wherein the method is used for determining whether a subject is at risk for developing loss of vision.

21. The method of claim 6 wherein the method is used for determining whether a subject is at risk for requiring future corneal transplant.

22. The method of claim 6 wherein the method is used for determining whether a subject is at risk for developing SCCD.
